SEQUENCE LISTING (56 sequences)

SEQ ID NO: 1 - protein, 18 aa, artificial sequence
  skipping sequence
EGRGSLLTCGDVEENPGP

SEQ ID NO: 2 - protein, 19 aa, artificial sequence
  skipping sequence
ATNFSLLKQAGDVEENPGP

SEQ ID NO: 3 - protein, 20 aa, artificial sequence
  skipping sequence
QCTNYALLKLAGDVESNPGP

SEQ ID NO: 4 - protein, 22 aa, artificial sequence
  skipping sequence
VKQTLNFDLLKLAGDVESNPGP

SEQ ID NO: 5 - DNA, 20 bp, artificial sequence
  guide RNA per DNA sequence
gttcaagaagcacacgactg

SEQ ID NO: 6 - DNA, 20 bp, artificial sequence
  guide RNA per DNA sequence
gttaactgctcactggatgg

SEQ ID NO: 7 - DNA, 20 bp, artificial sequence
  guide RNA per DNA sequence
ggagccggctgagagaagtt

SEQ ID NO: 8 - DNA, 20 bp, artificial sequence
  guide RNA per DNA sequence
gtggagataatctgtcctaa

SEQ ID NO: 9 - DNA, 20 bp, artificial sequence
  guide RNA per DNA sequence
agtccctatcccatcatcca

SEQ ID NO: 10 - DNA, 20 bp, artificial sequence
  guide RNA per DNA sequence
tgagcattgcagactaatct

SEQ ID NO: 11 - DNA, 20 bp, artificial sequence
  guide RNA per DNA sequence
aagtccctatcccatcatcc

SEQ ID NO: 12 - DNA, 20 bp, artificial sequence
  guide RNA per DNA sequence
tcttggatatttgtccctga

SEQ ID NO: 13 - DNA, 20 bp, artificial sequence
  guide RNA per DNA sequenc
gttgggaaataaactgtcta

SEQ ID NO: 14 - DNA, 20 bp, artificial sequence
  guide RNA per DNA sequence
ggtggataacgccctccaat

SEQ ID NO: 15 - DNA, 20 bp, artificial sequence
  guide RNA per DNA sequence
gtggataacgccctccaatc

SEQ ID NO: 16 - DNA, 20 bp, artificial sequence
  guide RNA per DNA sequence
ctgggagttacccgattgga

SEQ ID NO: 17 - DNA, 20 bp, artificial sequence
  guide RNA per DNA sequence
cctccaatcgggtaactccc

SEQ ID NO: 18 - DNA, 20 bp, artificial sequence
  guide RNA per DNA sequence
atccaccttccactgtactt

SEQ ID NO: 19 - DNA, 20 bp, artificial sequence
  guide RNA per DNA sequence
ttcaactgctcatcagatgg

SEQ ID NO: 20 - DNA, 20 bp, artificial sequence
  guide RNA per DNA sequence
gatttcaactgctcatcaga

SEQ ID NO: 21 - DNA, 20 bp, artificial sequence
  guide RNA per DNA sequence
tgggatagaagttattcagc

SEQ ID NO: 22 - DNA, 20 bp, artificial sequence
  guide RNA per DNA sequence
attcagcaggcacacaacag

SEQ ID NO: 23 - DNA, 20 bp, artificial sequence
  guide RNA per DNA sequence
ggccaaagtacagtggaagg

SEQ ID NO: 24 - DNA, 20 bp, artificial sequence
  guide RNA per DNA sequence
gtcctcggggcatgttccga

SEQ ID NO: 25 - DNA, 20 bp, artificial sequence
  uide RNA per DNA sequence
tcctcggggcatgttccgag

SEQ ID NO: 26 - DNA, 20 bp, artificial sequence
  guide RNA per DNA sequence
aggcatcggaaaatccacag

SEQ ID NO: 27 - DNA, 20 bp, artificial sequence
  guide RNA per DNA sequence
ctcaggttgggtgcgtctga

SEQ ID NO: 28 - DNA, 20 bp, artificial sequence
  guide RNA per DNA sequence
acgagatgcctgaacaaacc

SEQ ID NO: 29 - DNA, 20 bp, artificial sequence
  guide RNA per DNA sequence
acctgagtcccattttccaa

SEQ ID NO: 30 - DNA, 20 bp, artificial sequence
  guide RNA per DNA sequence
tcagccatcactaagacccc

SEQ ID NO: 31 - DNA, 20 bp, artificial sequence
  guide RNA per DNA sequence
caaaccaggggtcttagtga

SEQ ID NO: 32 - DNA, 20 bp, artificial sequence
  guide RNA per DNA sequence
ctaagacccctggtttgttc

SEQ ID NO: 33 - DNA, 20 bp, artificial sequence
  guide RNA per DNA sequence
tcaggcatctcgtccaaatg

SEQ ID NO: 34 - DNA, 20 bp, artificial sequence
  guide RNA per DNA sequence
aagacacaggttttcatgtt

SEQ ID NO: 35 - DNA, 20 bp, artificial sequence
  guide RNA per DNA sequence
gggctcattatcagttgacg

SEQ ID NO: 36 - DNA, 20 bp, artificial sequence
  guide RNA per DNA sequence
ggtcttctagacgtttaagt

SEQ ID NO: 37 - DNA, 2868 bp, artificial sequence
  gBlock sequence
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgagtttatcggccctgggaccaaagtggatatcaaacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 38 - DNA, 2347 bp, artificial sequence
  gBlock sequence
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagggaagcggagagggcagaggaagtctgctaacatgcggtgacgtcgaggagaatcctggacctatgggatggtcatgtatcatcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgagtttatcggccctgggaccaaagtggatatcaaacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgagatga

SEQ ID NO: 39 - DNA, 2910 bp, artificial sequence
  gBlock sequence
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattcttcctatgtgcgcccgctgtcagtggccctgggggagacggccaggatttcctgtggacgacaggcccttggaagtagagctgttcagtggtatcaacataggccaggccaggcccctatattgctcatttataataatcaagaccggccctcagggatccctgagcgattctctggcacccctgatattaattttgggaccagggccaccctgaccatcagcggggtcgaagccggggatgaagccgactattactgtcacatgtgggatagtagaagtggcttcagttggtctttcggcggggcgaccaggctgaccgtcctacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtgcagctgcaggagtcgggcccaggactggtgaaaccttcggagaccctgtccgtcacctgcagtgtctctggagattccatgaataattactactggacttggatccggcagtcccccggaaagggactggagtggataggctatatctctgacagagaatcagcgacttacaacccctccctcaatagtcgagtcgtcatatcacgagacacgtcgaaaaaccaattgtccctaaaattaaactccgtcacccctgcggacacggccgtctattactgtgcgacagcgcgccgaggacagaggatttatggagtggtttcctttggagagttcttctactactactccatggacgtctggggcaaggggaccacggtcaccgtctcctcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 40 - DNA, 2856 bp, artificial sequence
  gBlock sequence
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattctgacatccagatgacccagtctccatcctccctgtctgcctctgtgggagataccgtcactatcacttgccaggcaaacggctacttaaattggtatcaacagaggcgagggaaagccccaaaactcctgatctacgatgggtccaaattggaaagaggggtcccatcaaggttcagtggaagaagatgggggcaagaatataatctgaccatcaacaatctgcagcccgaagacattgcaacatatttttgtcaagtgtatgagtttgtcgtccctgggaccagactggatttgaaacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccaattgttacagtctggggcagcggtgacgaagcccggggcctcagtgagagtctcctgcgaggcttctggatacaacattcgtgactactttattcattggtggcgacaggccccaggacagggccttcagtgggtgggatggatcaatcctaagacaggtcagccaaacaatcctcgtcaatttcagggtagagtcagtctgactcgacacgcgtcgtgggactttgacacattttccttttacatggacctgaaggcactaagatcggacgacacggccgtttatttctgtgcgcgacagcgcagcgactattgggatttcgacgtctggggcagtggaacccaggtcactgtctcgtcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 41 - DNA, 2901 bp, artificial sequence
  gBlock sequence
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattcttccgatatatctgtggccccaggagagacggccaggatttcctgtggggaaaagagccttggaagtagagctgtacaatggtatcaacacagggccggccaggccccctctttaatcatatataataatcaggaccggccctcagggatccctgagcgattctctggctcccctgactccccttttgggaccacggccaccctgaccatcaccagtgtcgaagccggggatgaggccgactattactgtcatatatgggatagtagagttcccaccaaatgggtcttcggcggagggaccacgctgaccgtgttacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcagatgcagttacaggagtcgggccccggactggtgaagccttcggaaaccctgtccctcacgtgcagtgtgtctggtgcctccataagtgacagttactggagctggatccggcggtccccagggaagggacttgagtggattgggtatgtccacaaaagcggcgacacaaattacagcccctccctcaagagtcgagtcaacttgtcgttagacacgtccaaaaatcaggtgtccctgagccttgtggccgcgaccgctgcggactcgggcaaatattattgcgcgagaacactgcacgggaggagaatttatggaatcgttgccttcaatgagtggttcacctacttctacatggacgtctggggcaatgggactcaggtcaccgtctcctcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 42 - DNA, 2702 bp, artificial sequence
  gBlock sequence
gcatagctagcgctcttcaaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgacagcgggacgcaagtagtgagggcactcagaacgccactcagccccgacaggcagggcacgaggaggcagctcctcaccctccctttctcttttgtcctgcgggtcctcagggagttagaacttgtttattgcagcttataatggttacaaataaagcaatagcatcacaaatttcacaaataaagcatttttttcactgcattctagttgtggtttgtccaaactcatcaatgtatcttatcatgtctggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagtgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatgggatggtcatgtatcatcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgagtttatcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgctctccaaagcggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcaggtgagtcctcacaacctctctcctgctttaactctgaagggttttgctgctggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgactcgaggaatt

SEQ ID NO: 43 - DNA, 2744 bp, artificial sequence
  gBlock sequence
gcatagctagcgctcttcaaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgacagcgggacgcaagtagtgagggcactcagaacgccactcagccccgacaggcagggcacgaggaggcagctcctcaccctccctttctcttttgtcctgcgggtcctcagggagttagaacttgtttattgcagcttataatggttacaaataaagcaatagcatcacaaatttcacaaataaagcatttttttcactgcattctagttgtggtttgtccaaactcatcaatgtatcttatcatgtctggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagtgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatgggatggtcatgtatcatcctttttctagtagcaactgcaaccggtgtacattcttcctatgtgcgcccgctgtcagtggccctgggggagacggccaggatttcctgtggacgacaggcccttggaagtagagctgttcagtggtatcaacataggccaggccaggcccctatattgctcatttataataatcaagaccggccctcagggatccctgagcgattctctggcacccctgatattaattttgggaccagggccaccctgaccatcagcggggtcgaagccggggatgaagccgactattactgtcacatgtgggatagtagaagtggcttcagttggtctttcggcggggcgaccaggctgaccgtcctacgaactgtggctgcaccatctgtcttcatcttcccgccatctgatgagcagttgaaatctggaactgcctctgttgtgtgcctgctgaataacttctatcccagagaggccaaagtacagtggaaggtggataacgctctccaaagcggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtgcagctgcaggagtcgggcccaggactggtgaaaccttcggagaccctgtccgtcacctgcagtgtctctggagattccatgaataattactactggacttggatccggcagtcccccggaaagggactggagtggataggctatatctctgacagagaatcagcgacttacaacccctccctcaatagtcgagtcgtcatatcacgagacacgtcgaaaaaccaattgtccctaaaattaaactccgtcacccctgcggacacggccgtctattactgtgcgacagcgcgccgaggacagaggatttatggagtggtttcctttggagagttcttctactactactccatggacgtctggggcaaggggaccacggtcaccgtctcctcaggtgagtcctcacaacctctctcctgctttaactctgaagggttttgctgctggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgactcgaggaatt

SEQ ID NO: 44 - DNA, 20 bp, artificial sequence
  primer
cctggccccattgttcctta

SEQ ID NO: 45 - DNA, 20 bp, artificial sequence
  primer
gcgtctcaggacctttgtct

SEQ ID NO: 46 - DNA, 20 bp, artificial sequence
  primer
aatgtctgagttgcccaggg

SEQ ID NO: 47 - DNA, 20 bp, artificial sequence
  primer
tgtcacagaggtggtcctga

SEQ ID NO: 48 - DNA, 20 bp, artificial sequence
  primer
atggctgcaaagagctccaa

SEQ ID NO: 49 - DNA, 20 bp, artificial sequence
  primer
ggaaaaagggtcagaggcca

SEQ ID NO: 50 - DNA, 20 bp, artificial sequence
  primer
tgccctgtgattatccgcaa

SEQ ID NO: 51 - DNA, 20 bp, artificial sequence
  primer
gagctggaggaccgcaatag

SEQ ID NO: 52 - DNA, 20 bp, artificial sequence
  primer
gccactctagggcctttgtt

SEQ ID NO: 53 - DNA, 20 bp, artificial sequence
  primer
agcttcaaggcactgaggtc

SEQ ID NO: 54 - DNA, 19 bp, artificial sequence
  primer
ctacatggacgtctggggc

SEQ ID NO: 55 - DNA, 20 bp, artificial sequence
  primer
ctgctctcatcaagaccggg

SEQ ID NO: 56 - protein, 4 aa, artificial sequence
  nuclear localization added to protein
RRKR

(19) United States
(12) Patent Application Publication
(10) Pub. No.: US 2023/0181636 A1
(43) Pub. Date: Jun. 15, 2023

(54) ANTIBODY GENE EDITING IN B LYMPHOCYTES AND CO-EXPRESSION OF CARGO PROTEIN

(71) Applicant: The Rockefeller University, New York, NY (US)

(72) Inventors: Harald HARTWEGER, New York, NY (US); Mila JANKOVIC, New York, NY (US); Michel NUSSENZWEIG, New York, NY (US)

(21) Appl. No.: 17/906,469

(22) PCT Filed: Mar. 18, 2021
(86) PCT No.: PCT/US2021/023026

§ 371 Date: Sep. 15, 2022

Related U.S. Application Data
(60) Provisional application No. 62/991,482, filed on Mar. 18, 2020.

(51) Int. Cl. A61K 35/17; C07K 16/42; C07K 14/705; C12N 15/90; C12N 15/11; C12N 9/22

(52) U.S. Cl. CPC A61K 35/17; C07K 16/4208; C07K 14/70503; C12N 15/907; C12N 15/11; C12N 9/22; A61K 39/00

(57) ABSTRACT

This disclosure provides modified B cells which produce heterologous antibodies and co-express cargo proteins. The modified B cells may be stimulated by binding of a cognate antigen to the heterologous antibodies. The B cells may be reduced or eliminated by contacting the heterologous antibody with an anti-idiotypic antibody. Methods of making, and using the modified B cells for prophylaxis and therapy for a variety of conditions are provided. The B cells are modified at an IgH locus, an IgK locus, and combinations thereof. Modified B cells maintain allelic exclusion.

### CROSS-REFERENCE TO RELATED APPLICATIONS

This application claims priority to U.S. provisional patent application no. 62/991,482, filed Mar. 18, 2020, the entire disclosure of which is incorporated herein by reference.

### STATEMENT REGARDING FEDERALLY SPONSORED RESEARCH

This invention was made with government support under grant nos. 1UM1AI100663 and R01AI129795 awarded by the National Institutes of Health. The government has certain rights in the invention.

### BACKGROUND

There is an ongoing an unmet need for improved compositions and methods that can be used for controllably expressing a protein of interest, including but not necessarily limited to therapeutic proteins, in an individual. Specifically, there is a need for compositions and methods that can be reversibly induced to express therapeutic proteins. The present disclosure is pertinent to this need.

### SUMMARY

This disclosure provides modified B cells which produce heterologous antibodies and co-express cargo proteins. Methods of making, and using the modified B cells for prophylaxis and therapy for disorders are provided. The disclosure includes all polynucleotides that encode the heterologous antibodies, and all segments thereof, and all polynucleotides that encode the cargo proteins, including but not limited to such polynucleotides when provided as one or more DNA repair templates, in expression vectors, and as recombined into chromosomes of the B cells.

The modified B cells can be activated or otherwise influenced by binding of an antigen to the heterologous antibody. The modified B cells may also be reduced or eliminated by binding of an anti-idiotypic antibody to the heterologous antibody.

The modified B cells are produced using homologous recombination that is facilitated in part using a clustered regularly interspaced short palindromic repeats (CRISPR)-Cas (CRISPR-associated proteins) system for cleavage of the chromosome that allows homologous recombination between homologous segments of the chromosome and the repair templates.

Any of the described DNA templates may be provided in single stranded form, prior to recombination. The recombined sequences can be present in a double stranded segment of the DNA after repair of the homologous recombination. In embodiments of the disclosure, a λ light chain locus is disrupted or deleted.

Constructs and methods of making the described modified B cells are illustrated in FIGS. 5A, 5B and 5C. Each DNA repair template comprises 5′ and 3′ homology arms that are used for homologous recombination into a chromosome into the described loci. The DNA repair templates may comprise any combination of sequences that are depicted in each of FIGS. 5A, 5B, and 5C, and other figures of this disclosure.

In one embodiment, such as represented by FIG. 5A, the disclosure provides a single DNA template for homologous recombination into a chromosome. In an embodiment, this template comprises a single contiguous DNA sequence encoding a heterologous antibody, the heterologous antibody comprising a variable light chain region, an antibody light chain constant region, and an antibody variable heavy chain region. The sequence is introduced into an IgH locus in a B cell. This sequence also comprises a sequence encoding a cargo protein that is co-expressed with the heterologous antibody from a polycistronic element that also encodes the cargo protein, a variable light chain region, a light chain constant region and a variable heavy chain region. This configuration includes having a λ light chain locus deleted or disrupted. This configuration also includes disrupting or deleting the IgK locus in the B cells.

In another embodiment, such as represented by FIG. 5B, the disclosure provides a first and second contiguous DNA sequence for homologous recombination into a chromosome. The first contiguous sequence encodes a heterologous antibody, the heterologous antibody comprising a variable light chain region, a light chain constant region and a variable heavy chain region, that is introduced into the IgH locus. This embodiment also includes a second contiguous DNA sequence for homologous recombination into a chromosome that encodes a cargo protein. This second contiguous sequence encoding the cargo protein is introduced into the IgK locus.

In another embodiment, such as represented by FIG. 5C, the disclosure provides a first and second contiguous DNA sequences for homologous recombination into a chromosome. This approach includes a first contiguous DNA sequence for recombination into the IgH locus, and includes an SV40 polyadenylation signal or similar sequence that is used to stop translation from the 5′ endogenous Vh promoter of the endogenously rearranged variable region, a V_{H} promoter, a heavy chain LVDJ sequence, and a J_{H}H splice donor. This can be introduced into a segment of the IgH locus that is between a J_{H} and an Eμ enhancer segment. The second contiguous DNA segment is inserted in the IgK locus, which may be downstream (e.g., 3′) of a Jk5 segment, and before an iEκ enhancer segment. The second contiguous DNA sequence includes an SV40 polyadenylation or similar signal, a Vκ promoter, a sequence encoding the cargo protein, a P2A or similar site, a light chain LVJ segment, and a Jκ splice donor.

As will be apparent from the detailed description and the figures or the disclosure, the constructs and methods described above can include any one or a combination of: splice acceptors, splice donors, ribosome skipping sequences, polyadenylation sites, amino acid linkers, and a Cκ segments.

Populations of the described modified B cells are provided, which can include but are not limited to isolated populations. Plasma cells differentiated from the modified B cells are included.

Each single stranded DNA molecule comprising any of the described coding sequences and expression elements is included in the disclosure. Combinations of the single stranded DNA molecules are also included.

The disclosure includes all methods of making a modified B cells having the above described components.

The disclosure includes introducing the modified B cells into an individual, and administering an antigen that is cognate (e.g., with specificity binds) to the heterologous antibody. The disclosure includes cargo proteins expressed by the modified B cells that provide a prophylactic and/or therapeutic benefit to an individual, and thus also comprises using the modified B cells to provide the described benefits. The disclosure accordingly includes pharmaceutical compositions comprising the modified B cells. The disclosure also includes administering to the individual who received the modified B cells an anti-idiotypic antibody to stop or reduce production of the cargo protein. The anti-idiotypic antibody may reduce or eliminate the modified B cells from the individual.

### BRIEF DESCRIPTION OF THE FIGURES

FIG. 1. Efficient generation of indels in primary mouse B cells by CRISPR/Cas9. (A) Targeting scheme for Igh (crIgH) and Igk crRNA guides (crIgK₁, crIgK₂). (B) Experimental set up for (C-E). Primary mouse B cells were cultured for 24 h in the presence of anti-RP105 antibody and then transfected with Cas9 ribonucleoproteins (RNPs) and analyzed at the indicated time points. (C) Flow cytometric plots of cultured B cells at the indicated time points after transfection. Control uses an irrelevant crRNA targeting the HPRT gene. (D) Quantification of (C), percentage of Igκ⁻ Igλ− B cells by flow cytometry (right y-axis) and percentage of cells containing indels in the Igkc exon by TIDE analysis (left y-axis). Control bars include irrelevant HPRT-targeting crRNAs or a scramble crRNA without known targets in the mouse genome. (E) Percentage of cells containing indels in the J_{H}4 intron by TIDE analysis after targeting with crIgH or control. Bars indicate mean±SEM in two (TIDE) or four (flow cytometry) independent experiments.

FIG. 2. Engineering bNAb-expressing, primary, mouse B cells. (A) Schematic representation of the targeting strategy to create bNAb-expressing, primary mouse B cells. ssDNA homology-directed repair template (HDRT) contained 110 nt 5′ and 790 nt 3′ homology arms flanking an expression cassette. The 5′ homology arm is followed by the 111 nt long splice acceptor site and the first 2 codons of Cμ exon 1, a stop codon and a SV40 polyadenylation signal (CμSA SV40 pA). Then the mouse Ighv4-9 gene promoter, the leader, variable and joining regions (VJ) of the respective antibody light chain and mouse κ constant region (C_{κ}) are followed by a furin-cleavage site, a GSG-linker and a P2A self-cleaving oligopeptide sequence (P2A), the leader, variable, diversity and joining regions (VDJ) of the respective antibody heavy chain and 45 nt of the mouse J_{H}1 intron splice donor site to splice into downstream constant regions. (B) Experimental setup for (C). (C) Flow cytometric plots of primary, mouse B cells, activated and transfected with RNPs targeting the Ighj4 intron and Igkc exon with or without ssDNA HDRTs encoding the 3BNC60^{SI}, 3BNC117 or 10-1074 antibody. Non-transfected, antigen-binding B cells from 3BNC60^{SI} knock-in mice cultured the same way are used as control for gating. (D) Quantification of (C). Each dot represents one transfection. Data from 7 independent experiments (B-D). (E) Experimental set up for (F-H) (F) Flow cytometric plots of primary, mouse B cells, activated and transfected using ssDNA HDRT encoding the antibodies 3BNC60^{SI}, 3BNC117, PGT121 or 10-1074. B cells were expanded on feeder cells for 3 days. Cultured, non-transfected, antigen-binding B cells from PGT121 knock-in mice are shown for gating. (G) Quantification of (F). (H) Total number of antigen binding B cells before (24 h) or after 3 days (day 4) of feeder culture. Bars indicate mean±SEM. Combined data from 2 independent experiments for (E-H).

FIG. 3. Engineering bNAb-expressing, primary, human B cells. (A) Schematic representation of the targeting strategy to create bNAb-expressing, primary human B cells. The ssDNA HDRT is flanked by 179 nt and a 521 nt homology arms. The central expression cassette contains 112 nt of the human splice acceptor site and the first 2 codons of Cμ exon 1, a stop codon and a SV40 polyadenylation signal (CμSA SV40 pA). Then the human IGHV1-69 gene promoter, the leader, variable and joining regions (VJ) of the respective antibody light chain and human κ constant region (C_{κ}) are followed by a furin-cleavage site, a GSG-linker and a P2A self-cleaving oligopeptide sequence (P2A), the leader, variable, diversity and joining regions (VDJ) of the respective antibody heavy chain and 50 nt of the human J_{H}4 intron splice donor site to splice into downstream constant regions. (B) Experimental set up for (C, D). Primary human B cells were cultured for 24 h in the presence of anti-RP105 antibody and then transfected with RNPs±HDRT. (C) Flow cytometric plots of primary human B cells 48 h after transfection with RNPs containing crRNAs without target (scramble) or targeting the IGHJ6 intron or the IGKC exon. (D) Quantification of (C). Bars indicate mean±SEM. Combined data from 3 independent experiments is shown (B-D). (E) Flow cytometric plots of antigen binding by Igλ⁻ primary human B cells 72 h after transfection of RNPs targeting both the IGHJ6 intron and the IGKC exon with or without HDRTs encoding 3BNC60^{SI} or 10-1074. (F) Quantification of (E). Bars indicate mean±SEM. Combined data from 2 independent experiments with 2-4 replicates each (E, F).

FIG. 4. Engineered bNAb-expressing, primary, mouse B cells participate in humoral immune responses in vivo. (A) Experimental set up for (B-E). i.v. intravenous; i.p. intraperitoneal. (B) Anti-3BNC60^{SI} idiotype-coated, mouse IgG ELISA of sera from mice adoptively transferred with the indicated B cells and immunized with the cognate antigen TM4 core at the indicated time points. Representative plots of 7 independent experiments. (C) Anti-3BNC60^{SI} idiotype-coated, mouse IgG1^{a} or IgG1^{b} ELISA of day 14 sera, as above.

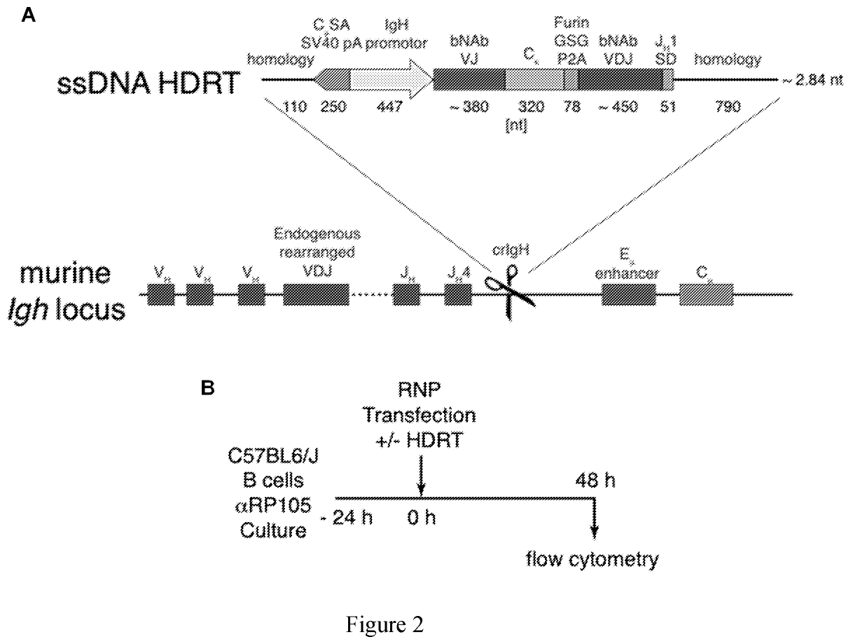

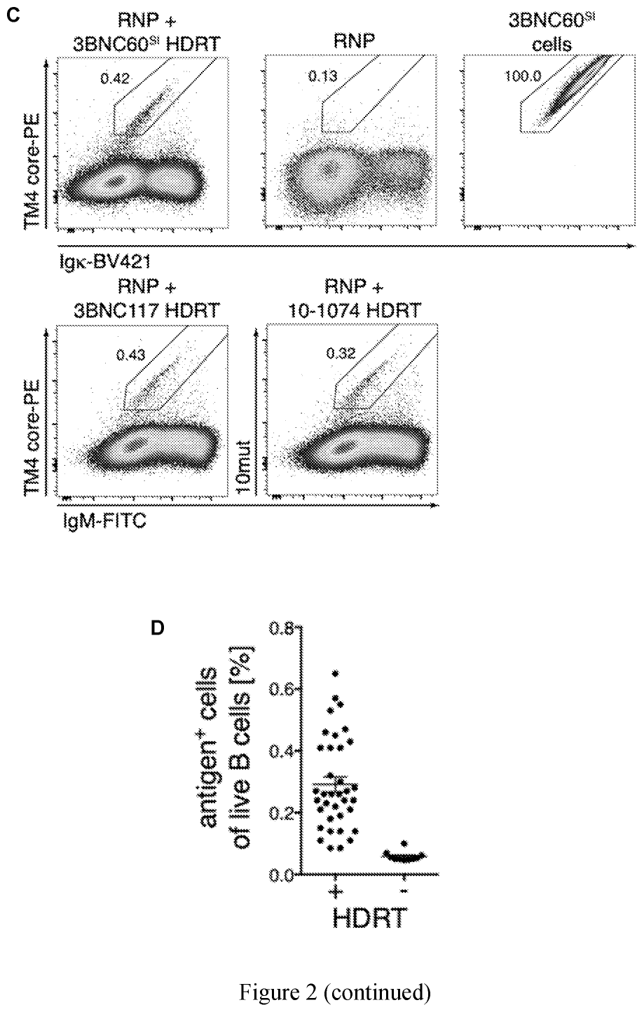

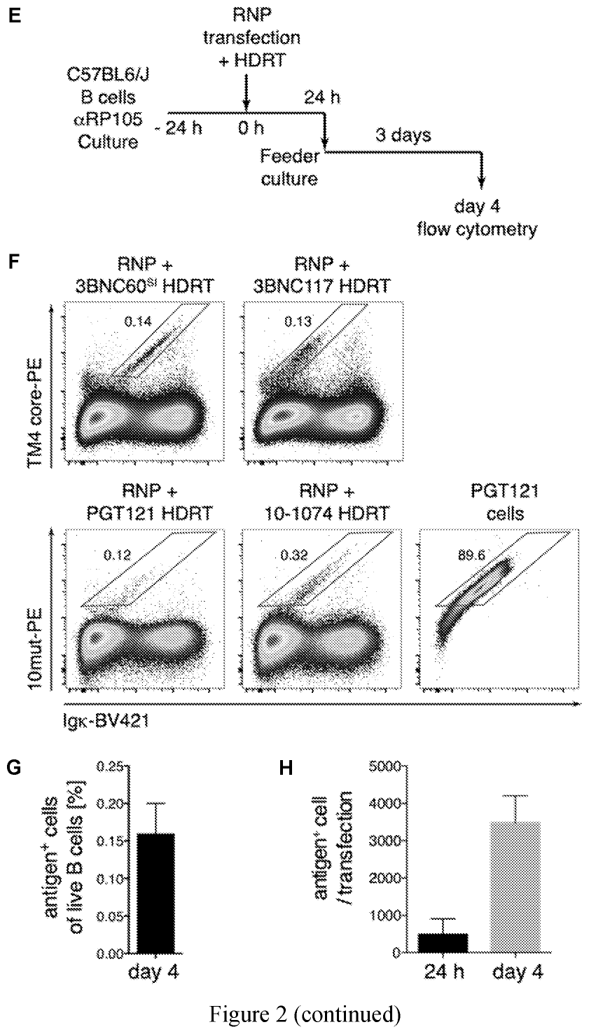

Representative plots of 2 independent experiments. (D) 3BNC60^{SI} serum IgG levels 14 days after immunization in mice transferred with 3BNC60^{SI}-edited cells, numbers of total B cells/mouse at transfection are indicated. Cells were transferred either 24 h after transfection or after additional culture on feeder cells as in FIG. 2 D. Determined by anti-3BNC60^{SI} idiotype-coated, mouse IgG ELISA over 7 independent experiments. Each dot represents one mouse and line indicates arithmetic mean. (E) TZM.b1 neutralization data of protein G-purified serum immunoglobulin days 14-21 after immunization from mice treated as in (A) but transfected with 10-1074 HDRT and immunized with cognate antigen 10mut. Combined data from 2 independent experiments are shown.

FIG. 5. Strategies to target a cargo such as a therapeutic protein to the IgH or IgK locus for expression. (A) Strategy to target cargo expression to the IgH locus and multiplexed ablation of the IgK locus. A polycistronic artificial exon is inserted between the last J gene and enhancer element allowing co-expression of cargo and antibody. (B) Strategy to target cargo expression to the IgKC locus and multiplexed insertion of an antibody into the IgH locus. Insertion of the cargo in the Cₖ exon will disrupt expression of the κ light chain and instead express the cargo. (C) Strategy to target cargo and antibody light chain expression to the IgKJ intron and multiplexed insertion of an antibody heavy chain into the IgH locus. Insertion of the cargo in the Cₖ exon will disrupt expression of the κ light chain and instead express the cargo. Simultaneous expression of a specific antibody allows in vivo activation and differentiation of edited B cells into plasma cells.

FIG. 6: Design and testing of AAV HDR donors for Cargo expression. (A) Design of a single AAV HDR donor that expresses the cargo from the IgHJ locus as a polycistronic transcript separated by 2A self-cleaving oligopeptide sequences. (B) Design of a single AAV HDR donor targeting 2 separate loci. Here the antibody is expressed from the IgHJ locus whereas the cargo is expressed from the IgKC locus which simultaneously disrupts endogenous kappa light chain expression. (C) Dual AAV targeting strategy with design of two AAV HDR donors targeting 2 separate loci for cargos larger than 2.4 kb. This design uses the same principle as the previous design but splits the targeting to the IGHJ and IgK locus into separate AAVs. (D) Both single AAV strategies can produce cargo and antibody simultaneously. Strategies were tested using the anti-4-hydroxy-3-nitrophenylacetyl (NP) specific B1-8^{hi} B cell receptor and mNeon fluorescent protein as cargo. Targeting only the IgH locus with a polycistronic template leads to co-expression of both mNeon and B1-8^{hi} BCR in edited cells but reduced expression of both compared to editing in only the B1-8^{hi} BCR or editing the B1-8 BCR into the IgHJ locus and the mNeon cargo into the IgKC locus. (E) Comparison of editing both the IgHJ and IgKC locus using a single or dual AAV strategies. Both strategies produce can produce high numbers of double edited cells however the single AAV strategy has a higher ratio of cells edited at both loci so is preferable if the cargo is sufficiently small. Expression levels of cargo and BCR are equally high for both strategies.

FIG. 7: Expression of functional coagulation factor 9 (FIX) using a single AAV targeting 2 loci. (A) Primary mouse B cells edited using strategy in FIG. 6B to express either human FIX or FLAG-tagged mouse FIX as cargo and the B1-8^{hi} BCR. Intracellular stain indicating expression of FIX and surface stain showing binding the B1-8^{hi} antigen NP. (B) Human FIX ELISA demonstrating that FIX is secreted into cell culture media. (C) Human FIX colorimetric activity assay demonstrating that human FIX produced by B cells is functional.

FIG. 8. Cultured B cells participate in humoral immune responses. (A) Schematic representation of the experimental set up for (B) and (C). B1-8^{hi} CD45.1 Igh^{a} cells were cultured for 24 or 48 h in the presence of anti-RP105 antibody, then rested for 2-3 h without antibody and then transferred into C57BL6/J (CD45.2 Igh^{b}) recipients. 18 h later, mice were immunized with NP-OVA i.p. and mice were analyzed 2 weeks later. (B) Flow cytometric plots gated on CD38⁻Fas⁺GL7⁺IgD⁻ GC B cells 2 weeks after transfer. (C) Pre-immune (day 0) and day 13 ELISA titers of anti-NP IgG1^{a} or IgG1^{b}. (D) Schematic representation of the experimental set up for (E). B1-8^{hi} CD45.1 Igh^{a} cells were cultured for 24 h and transfected with plasmid DNA. 24 h after transfection cells were transferred and analyzed as in (A). (E) Flow cytometric plots gated on CD38⁻Fas⁺GL7⁺IgD⁻ GC B cells 11 days after transfer. Data (A-E) are representative of 2-3 independent experiments.

FIG. 9. Identification of optimal mouse Igh crRNA and ssDNA HDRT template production. (A) Schematic representation of the mouse Igh locus around J_{H}4. Location and sequence of tested guide RNAs is indicated below. (B) TIDE assay comparing the efficiency of creating indels of the crRNAs indicated in (A). Forward/reverse indicate sequencing with forward/reverse primers respectively. Representative of 2 independent experiments. (C) Flow chart of ssDNA production. HDRT templates were cloned into pLSODN-4D, Maxi-prepped, sequence verified and digested with restriction enzyme XhoI and the nicking endonuclease Nt.BspQI to produce 3 ssDNA fragments of the vector. Denaturing loading buffer was used to separate the 3 fragments by conventional agarose gel electrophoresis as indicated. ssDNA HDRT quality and integrity was verified using (D) Bioanalyzer and (E) agarose gel electrophoresis. Representative of >20 independent preparations.

FIG. 10. Cell viability and Igh allelic exclusion of bNAb expressing murine B cells. (A) Flow cytometric plots showing percentage of live cells among all events 48 h after RNP±HDRT transfection. Related to FIG. 2 B, C. (B) Experimental set up for (C). Heterozygous (Igh′) B cells expressing IgH^{a} or IgH^{b} alleles were activated for 24 h, then transfected with 3BNC60^{SI} HDRT and analysed 48 h later. (C) Overlays of flow cytometric plots of TM4 core binding cells and non-binding B cells, both pre-gated on λ⁻ B cells. TM4 core mean fluorescence intensity (5.89×10³ to 1.28×10⁵) is color mapped onto TM4 core-binding cell population. Numbers represent the percentage of TM4 core-binding cells among λ⁻ B cells (left) or the percentage of TM4 core-binding B cells in the respective gate (right). Concatenate of 5 technical repeats in 2 independent experiments is shown (B-C). (D) Schematic representation of the promoterless targeting strategy to create bNAb-expressing, primary mouse B cells. ssDNA homology-directed repair template (HDRT) contained 110 nt 5′ and 790 nt 3′ homology arms flanking an expression cassette. The 5′ homology arm is followed by the 111 nt long splice acceptor site and the first 2 nucleotides of Cμ exon 1 and an in-frame T2A sequence with GSG linker. Then the leader, variable and joining regions (VJ) of the respective antibody light chain and mouse κ constant region (C_{κ}) are followed by a furin-cleavage site, a GSG-linker and a P2A self-cleaving oligopeptide sequence (P2A), the leader, variable, diversity and joining regions (VDJ) of the respective antibody heavy chain and 45 nt of the mouse J_{H}1 intron splice donor site to splice into downstream constant regions. (E) Flow cytometry of mouse B cells transfected and analysed as in FIG. 2 B either without template, or promoter-driven template or promoterless HDRT encoding 3BNC60^{SI}. Left panel shows cognate antigen binding (TM4 core) and right panel identifies correctly edited B cells using anti-idiotypic antibody iv8. (F) Geometric mean fluorescence intensity of TM4 core-binding of cells gated as in the left panel of (E). Bars indicate mean±SEM. Representative of 2 independent experiments.

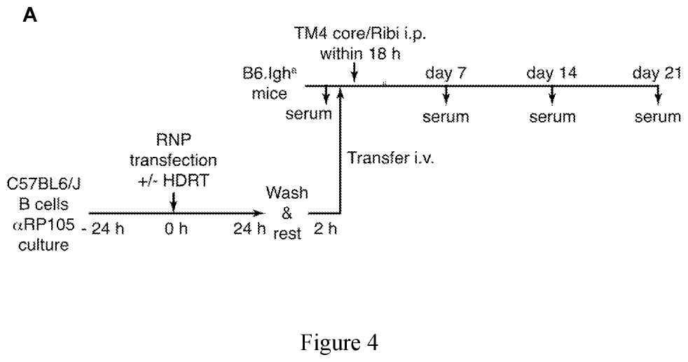

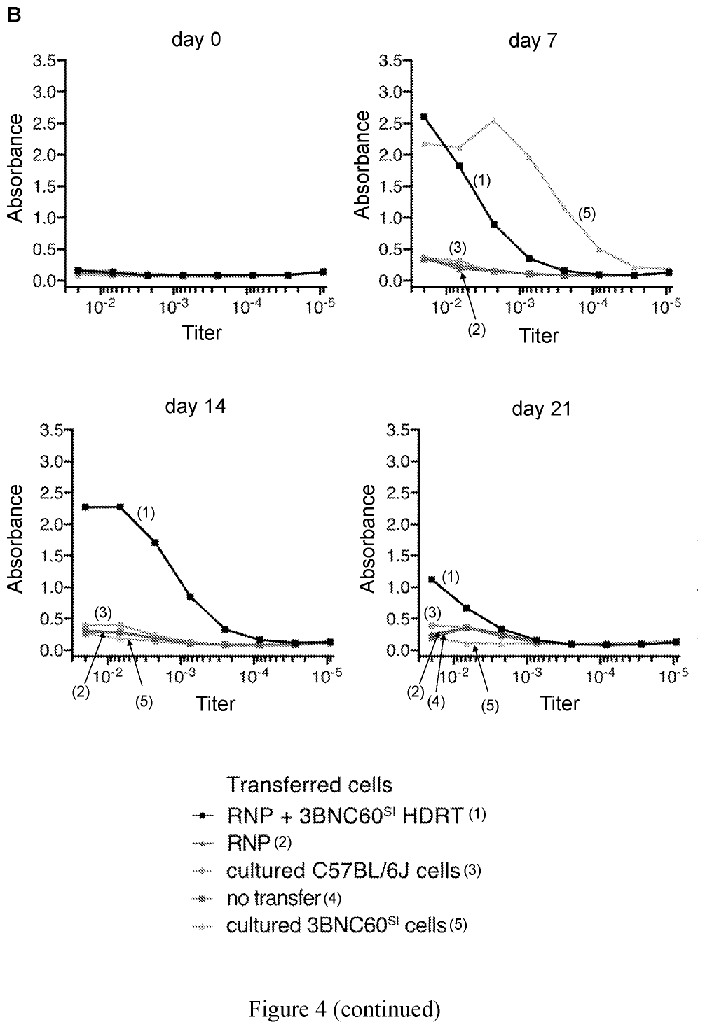

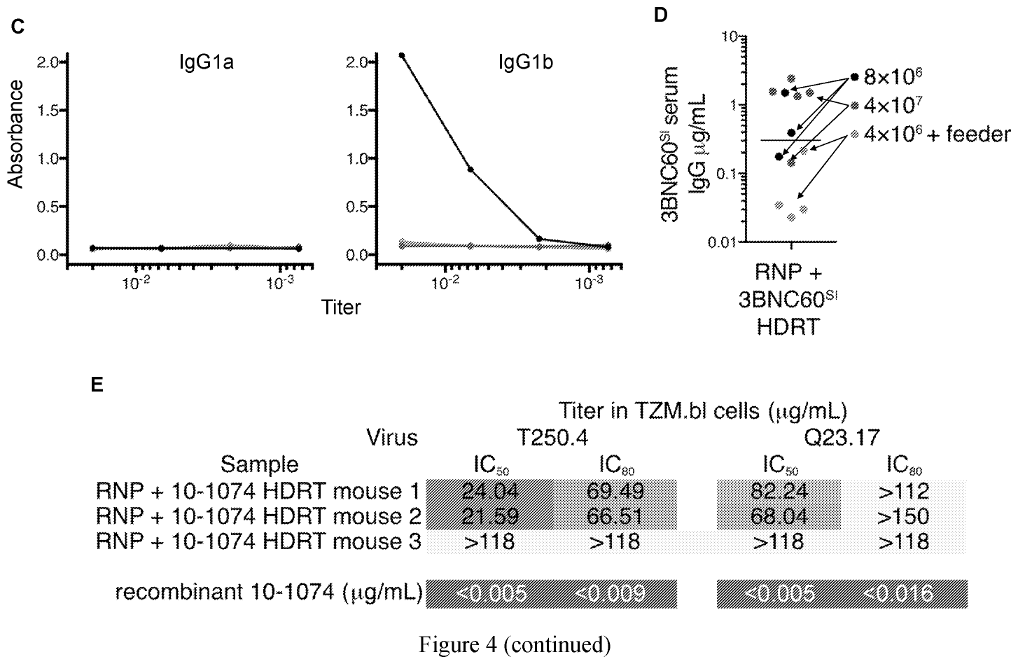

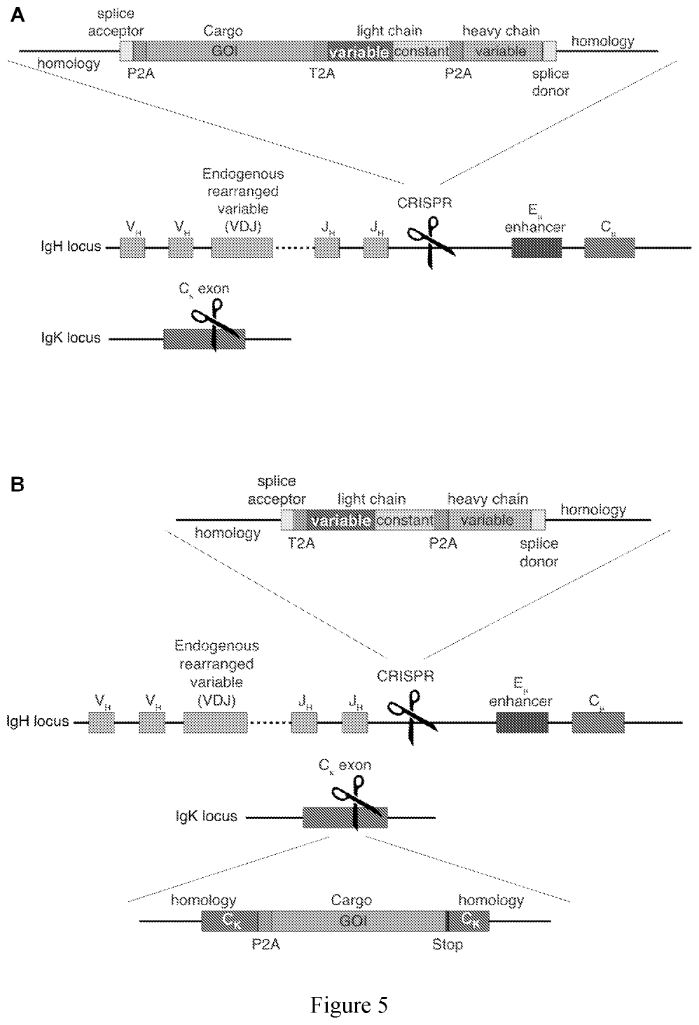

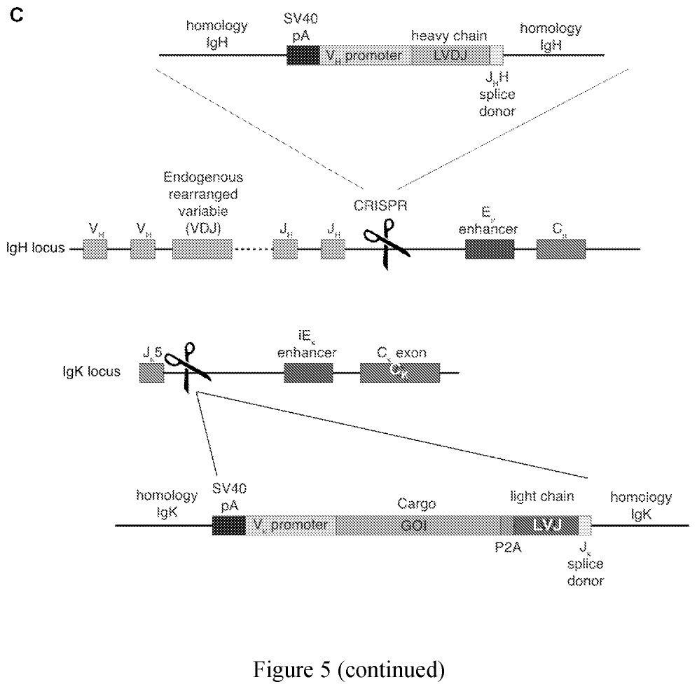

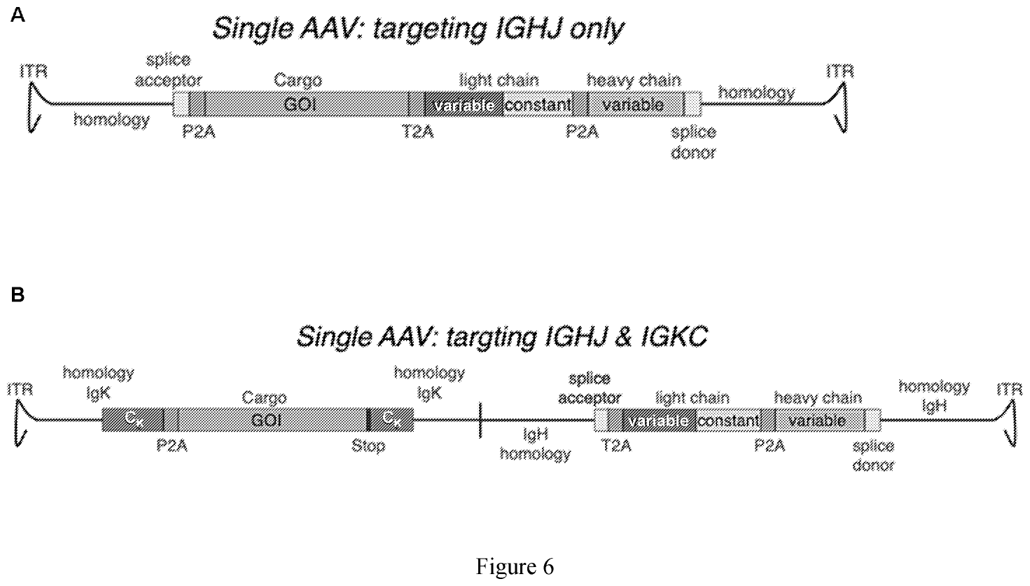

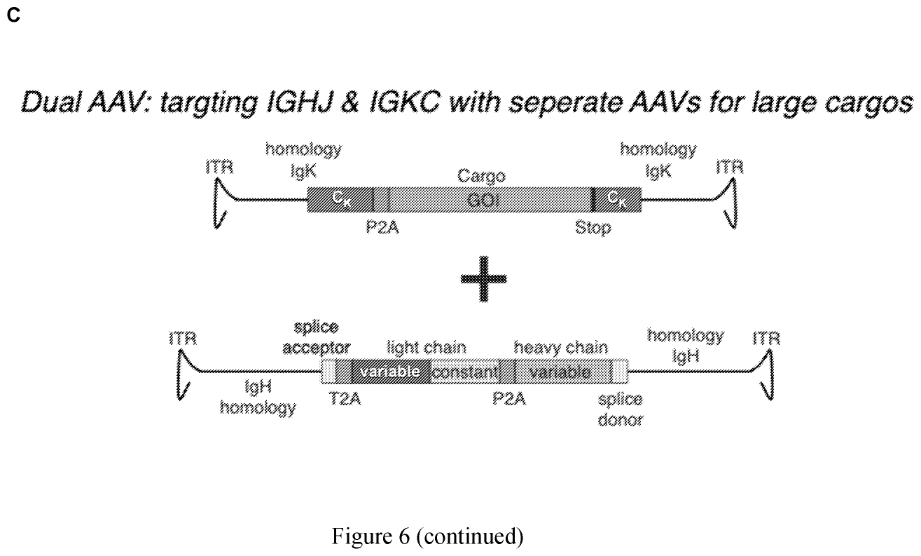

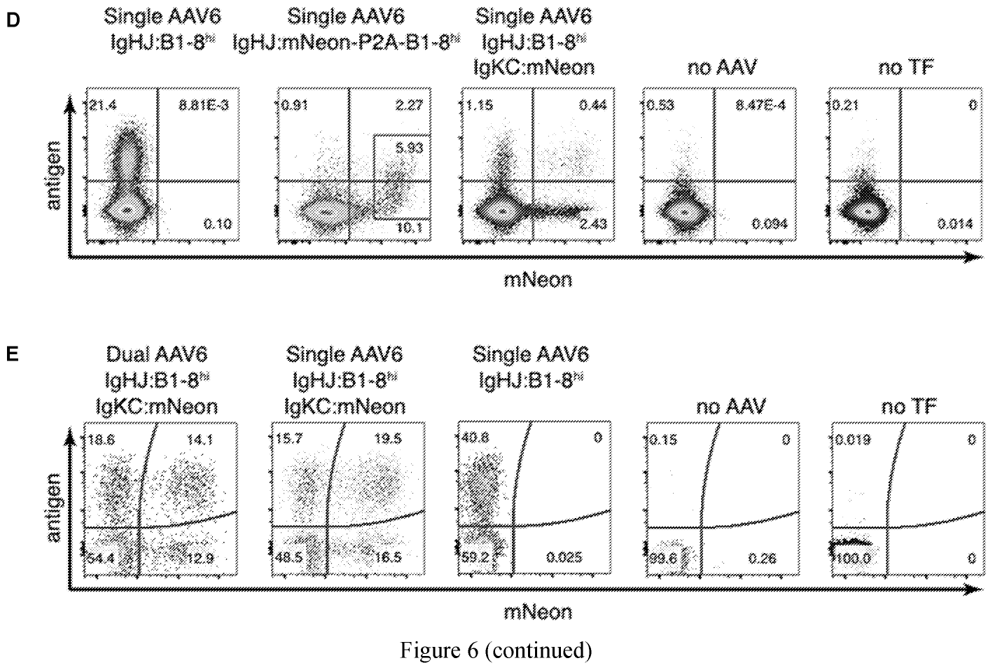

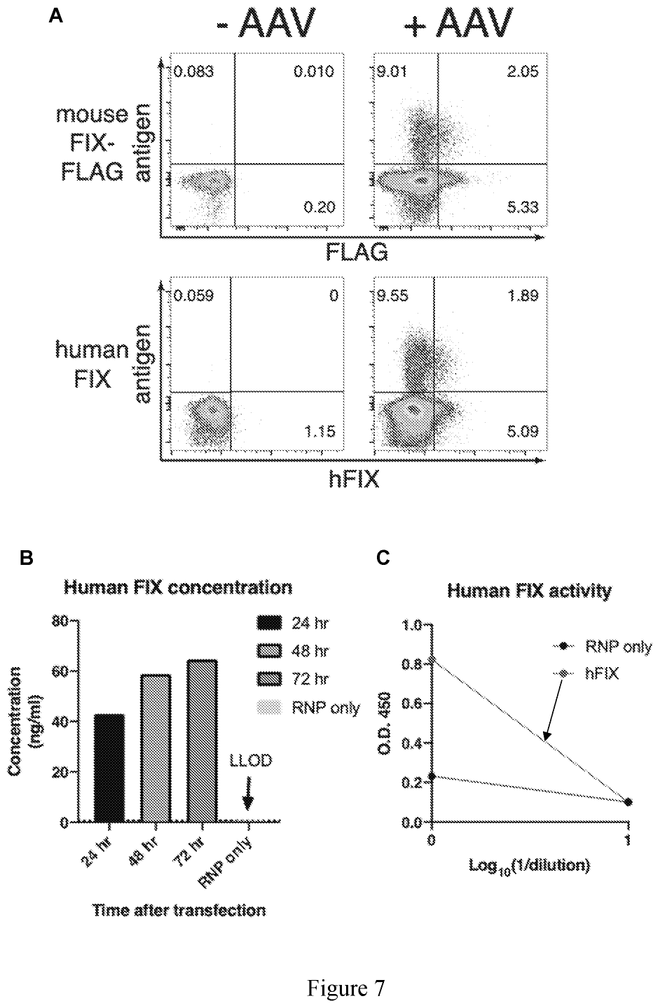

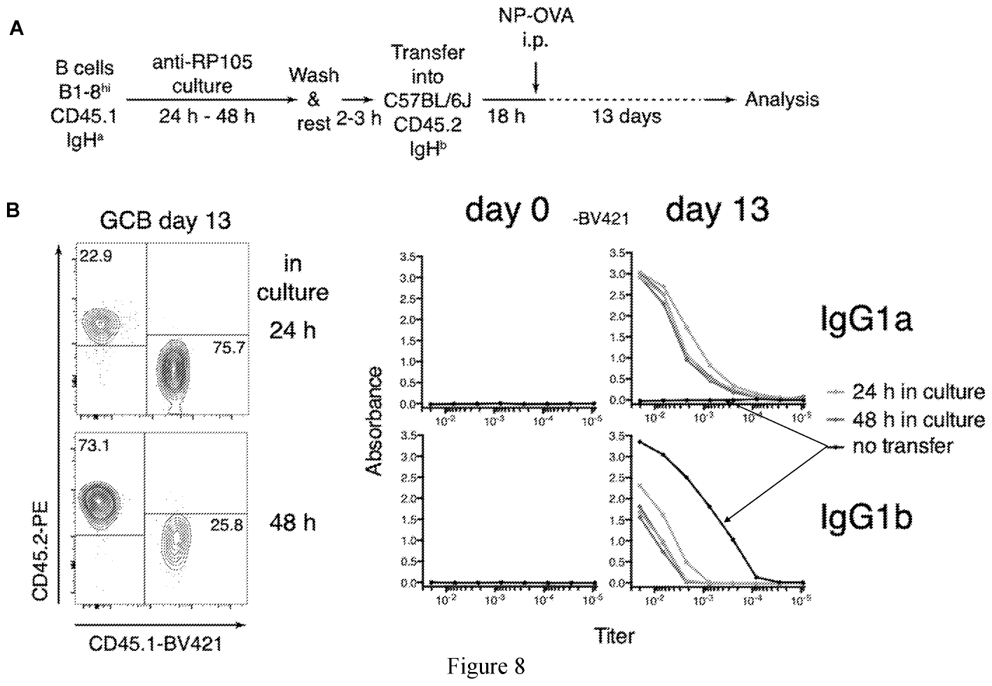

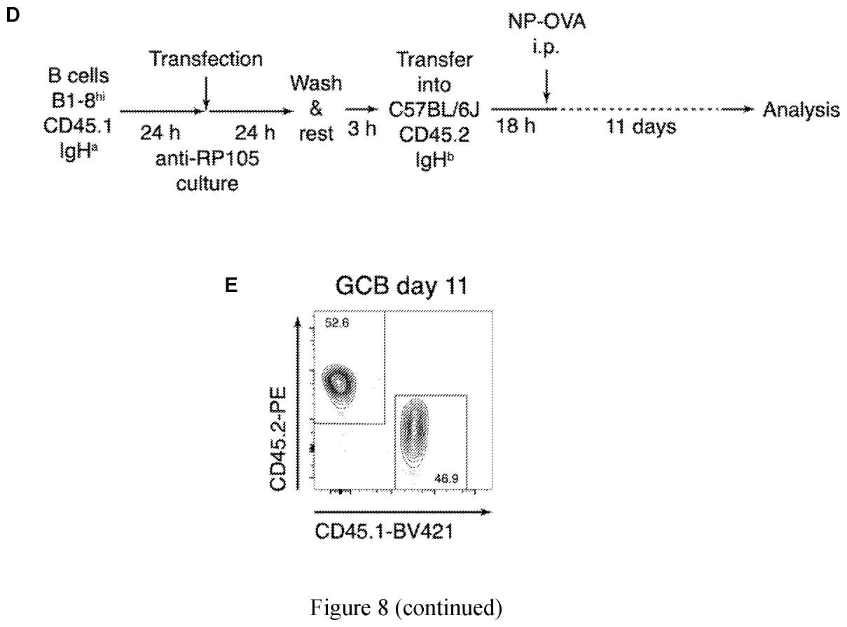

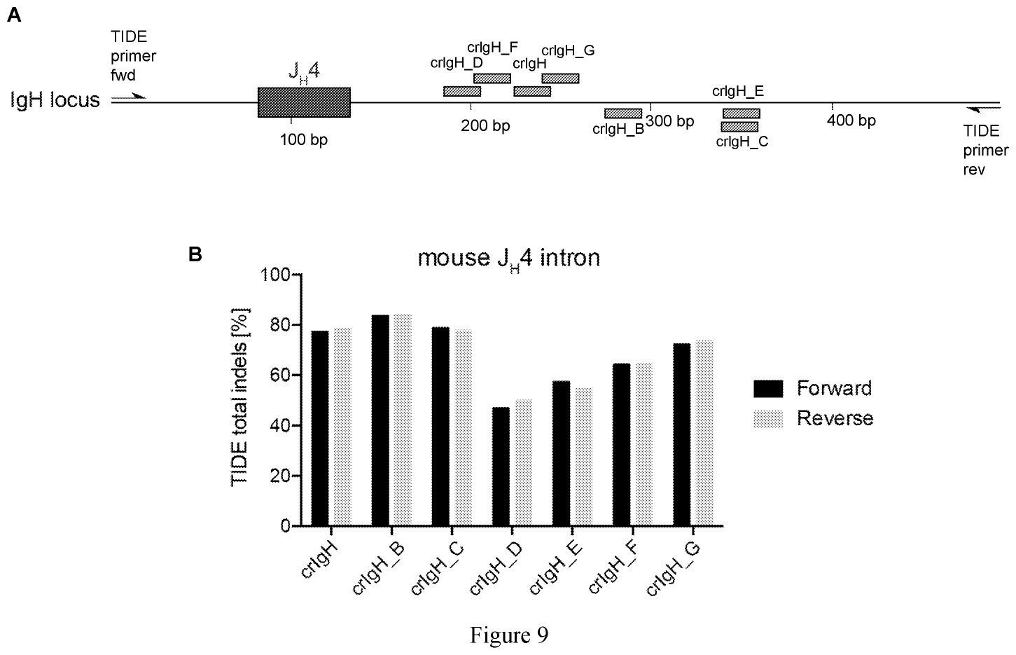

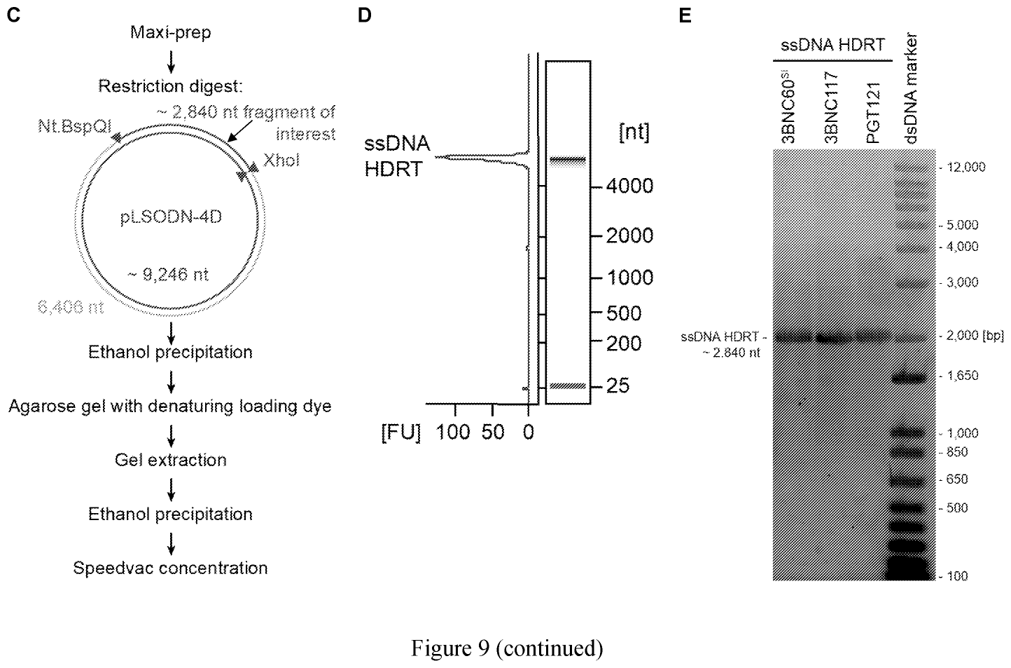

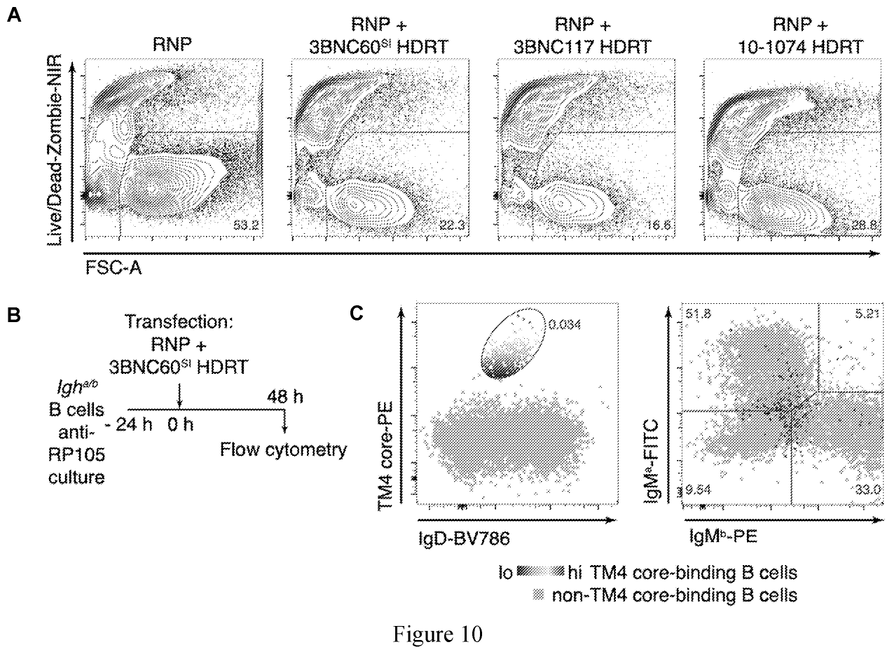

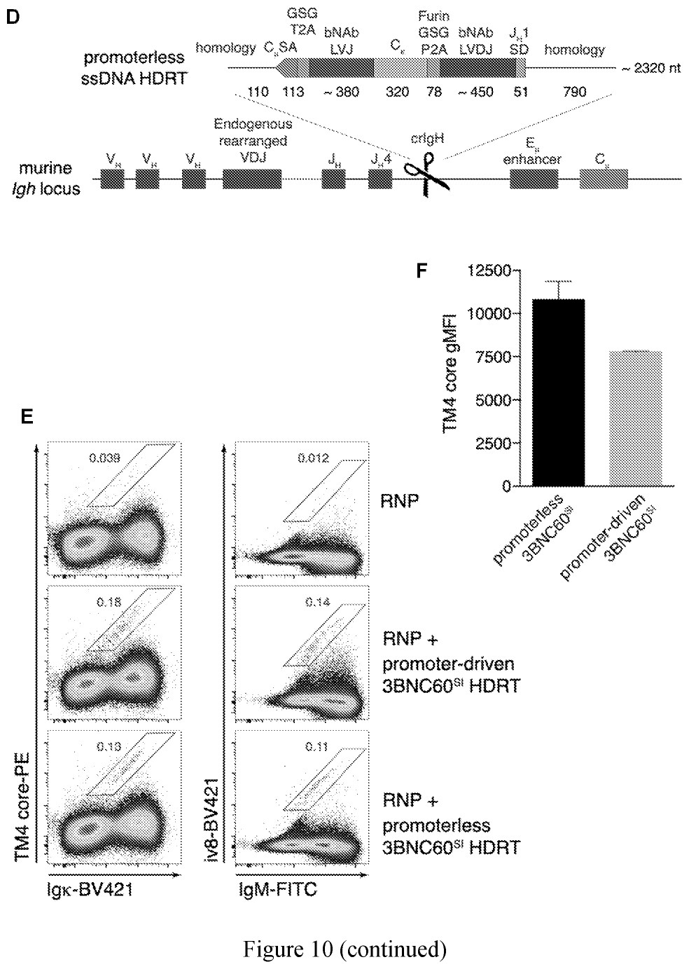

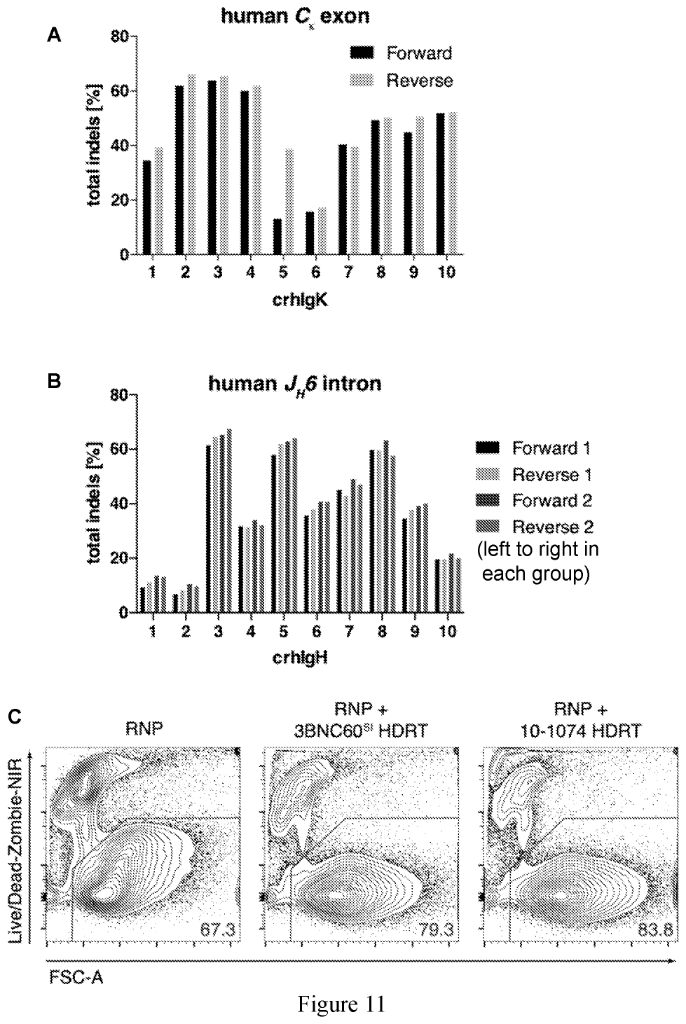

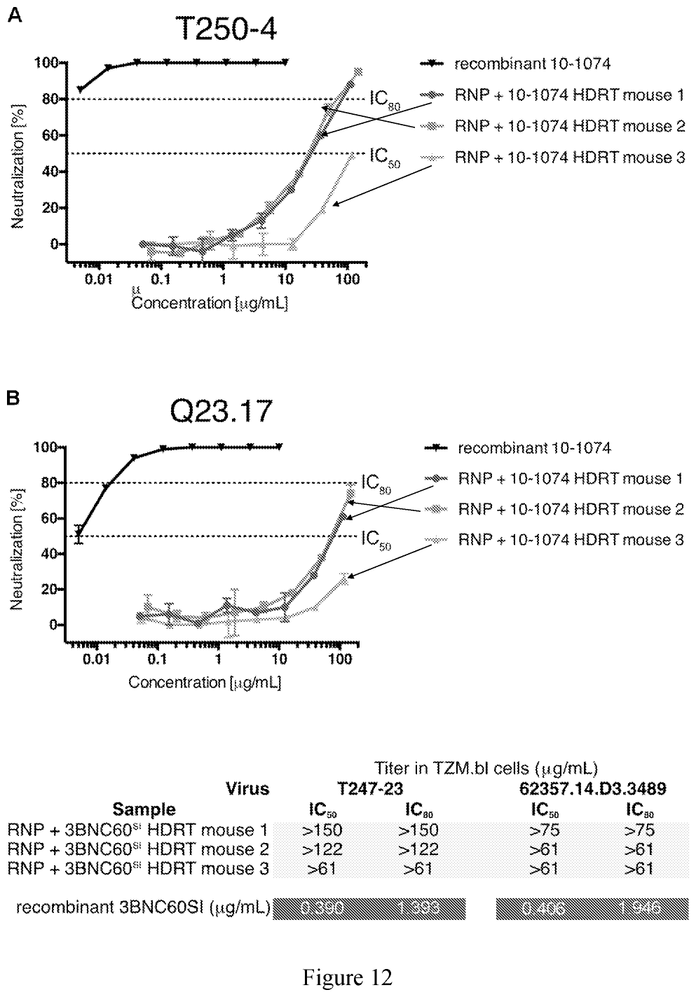

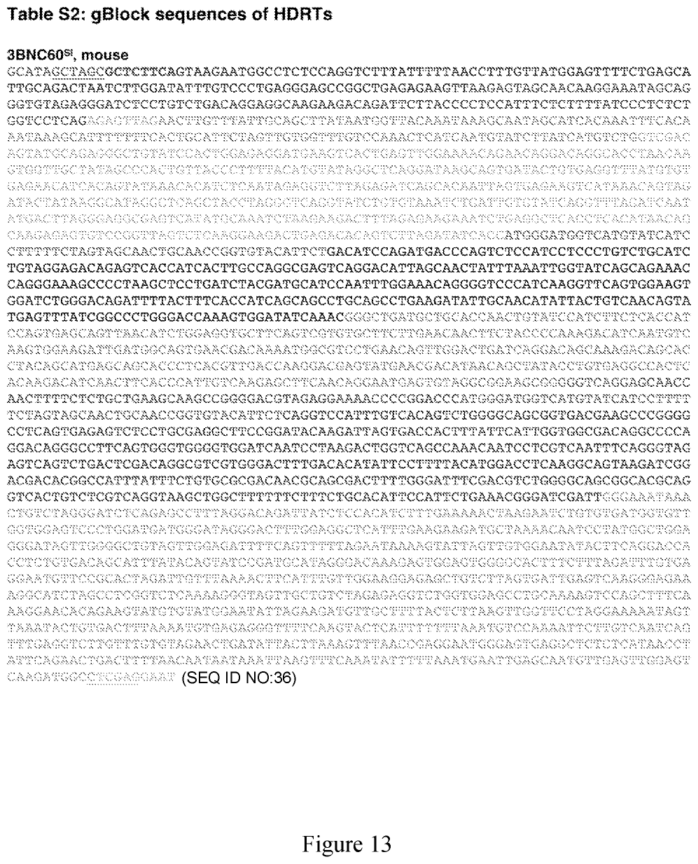

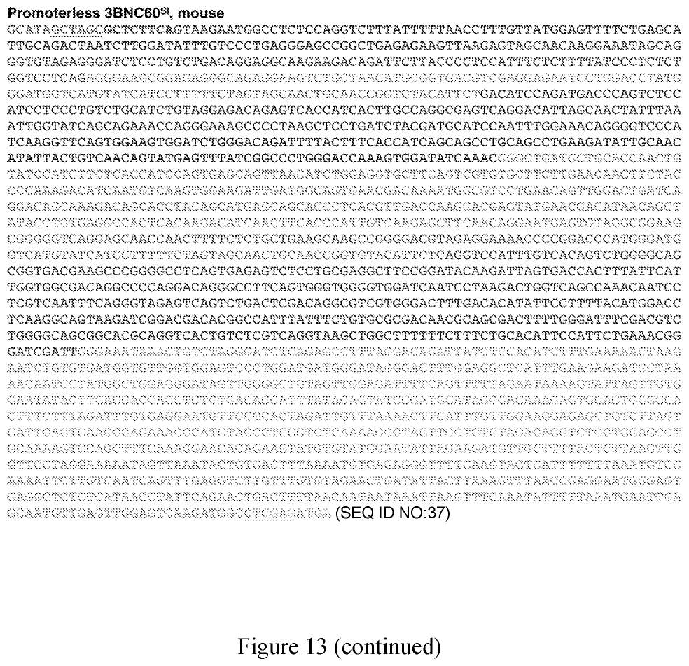

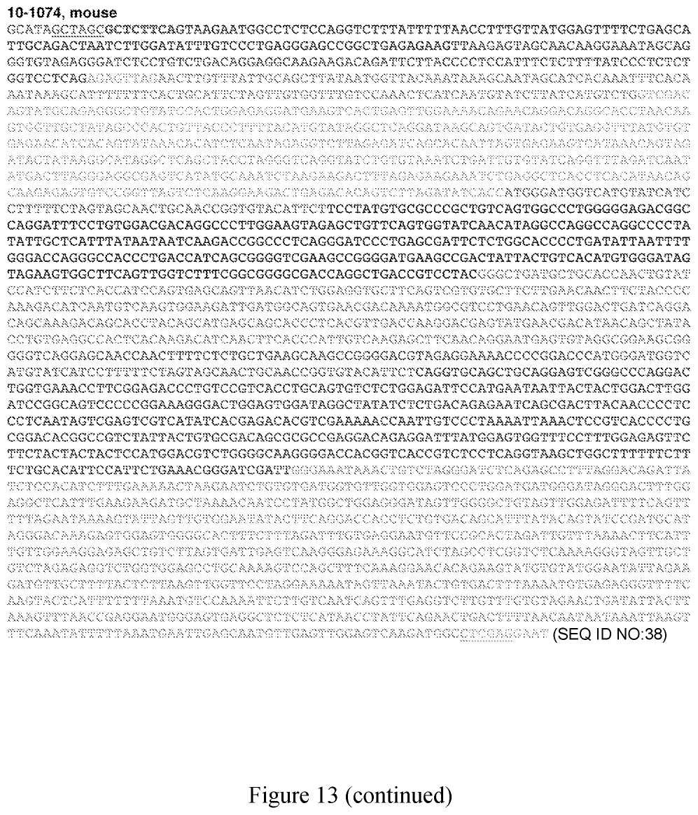

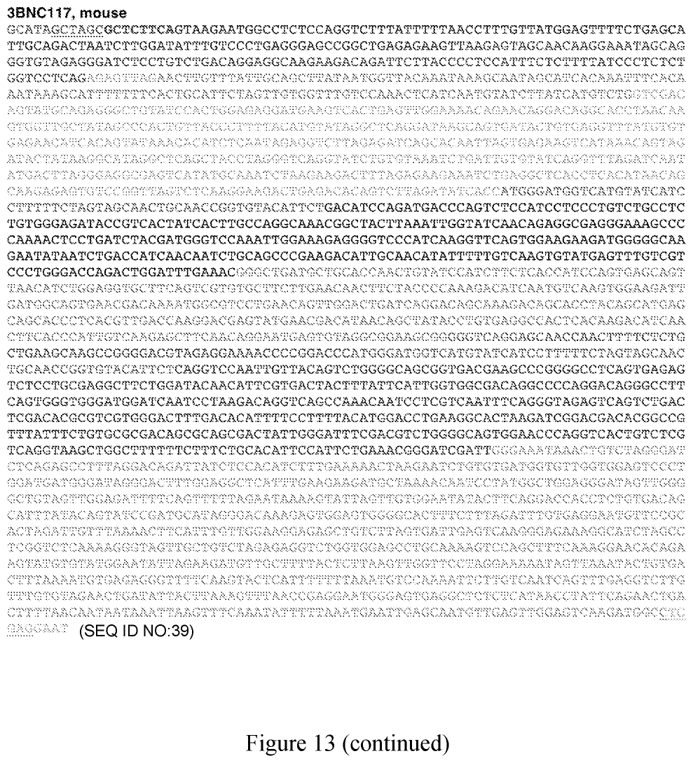

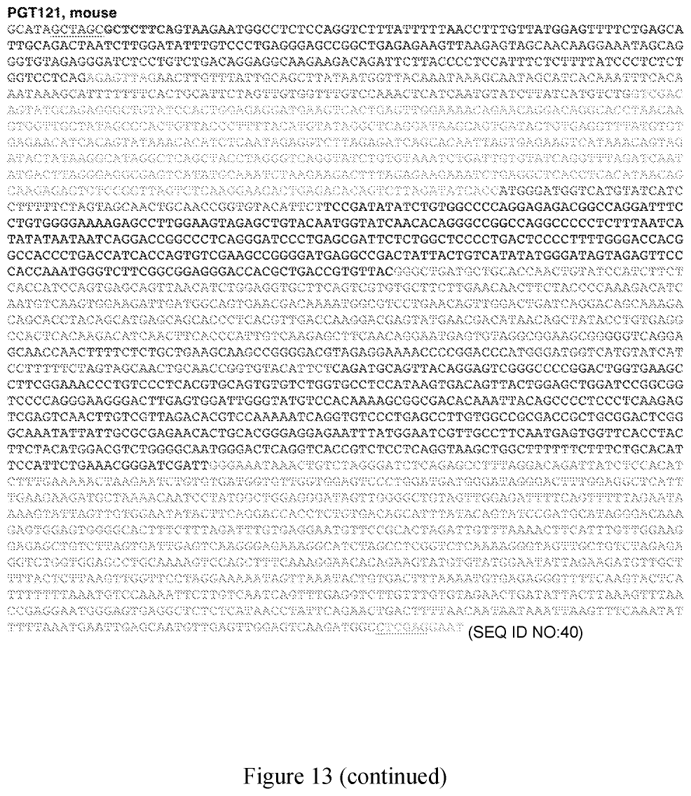

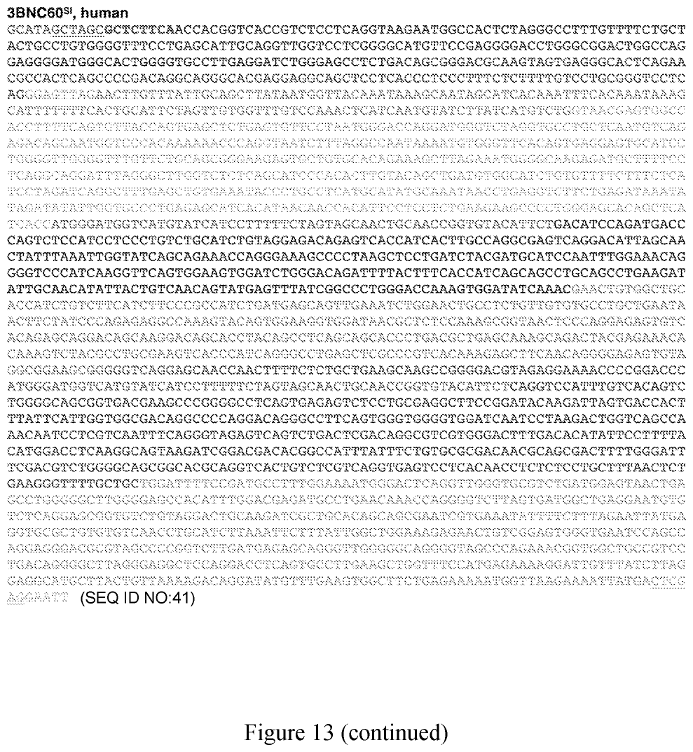

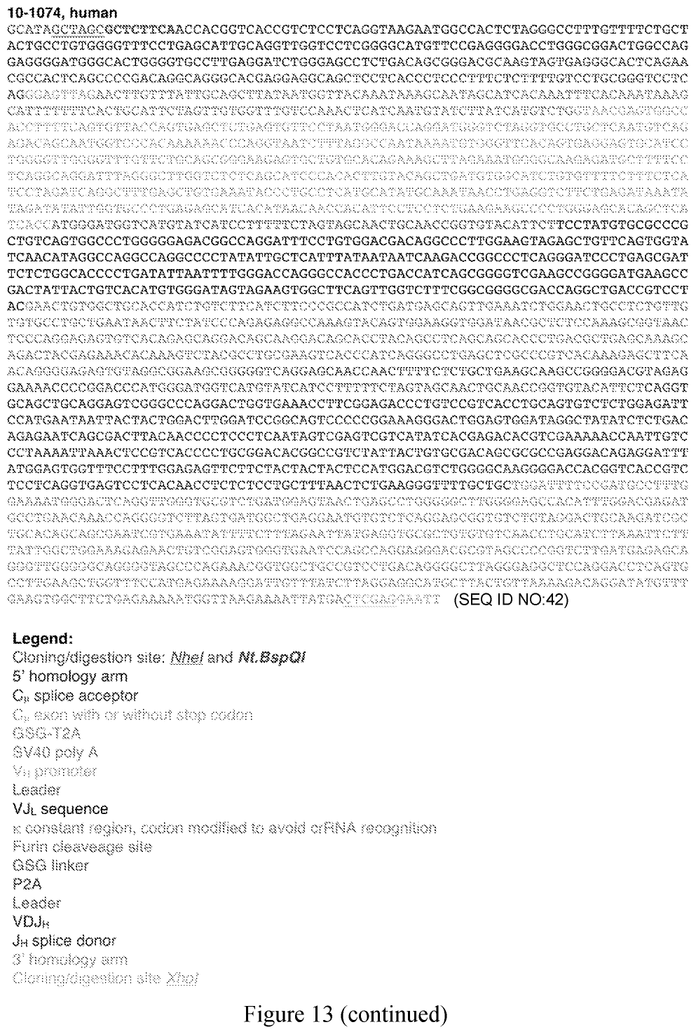

FIG. 11. TIDE analysis and viability of primary, human B cells after transfection. (A) TIDE assay 42 h after transfection, comparing the efficiency of creating indels of crRNAs targeting the human IGKC exon and (B) TIDE assay using 2 different primer sets, 24 h after transfection, comparing the efficiency of creating indels of crRNAs targeting the human IGHJ6 intron. Forward/reverse indicate sequencing with forward/reverse primers respectively. Representative of 2 independent experiments. (C) Flow cytometric plots showing percentage of live cells among all events 72 h after RNP±HDRT transfection. Related to FIG. 4 D. Representative plots of 2 independent experiments are shown.

FIG. 12. Serum neutralization of wild type mice adoptively transferred with edited B cells. Related to FIG. 4. (A, B) Neutralization curves for HIV strains T240-4 (A) and Q23.17 (B) of data summarized in FIG. 4 E of mice receiving 10-1074-edited B cells and immunized with cognate antigen 10mut. (C) HIV neutralization data of mice receiving 3BNC60^{SI}-edited B cells and immunized with cognate antigen TM4 core. Combined data from 2 independent experiments (A-C).

FIG. 13. Color annotated version of Table 2.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. All time intervals, temperatures, reagents, culture conditions and media, described herein are included in this disclosure.

The disclosure includes all steps and compositions of matter described herein in the text and figures of this disclosure, including all such steps individually and in all combinations thereof, and includes all compositions of matter including but not necessarily limited to vectors, cloning intermediates, cells, cell cultures, progeny of the cells, and the like. The disclosure includes all polynucleotide sequences, their RNA or DNA equivalents, all complementary sequences, and all reverse complementary sequences. If reference to a database entry is made for a sequence, the sequence is incorporated herein by reference as it exists in the database as of the filing date of this application or patent. This disclosure includes all nucleic acid and amino acid sequences described herein and all contiguous segments thereof including all integers and ranges of integers there between. In embodiments, each complementary determining region (CDR) amino acid sequence of each antibody of this disclosure is included as a distinct sequence.

Throughout this application, unless stated differently, the singular form encompasses the plural and vice versa. All sections of this application, including any supplementary sections or figures, are fully a part of this application

This disclosure relates in part to the disclosure in U.S. patent publication no. US-20210024889, published Jul. 24, 2019, the entire disclosure of which is incorporated herein by reference.

This disclosure provides modified B cells, heterologous antibodies made by such B cells, co-expressed proteins, vectors and cells comprising nucleic acids encoding the antibodies and co-expressed proteins, compositions comprising any of the foregoing, methods of making any of the foregoing, and methods of using the modified B cells expressing the antibodies in the treatment and/or prophylaxis of a condition associated with the antigen to which the produced antibodies bind with specificity, methods of inducing expression of the co-expressed protein, and methods of stopping the expression of the antibody and the co-expressed protein, such by controlled elimination of the modified B cells, as further described below, and at least by way of FIG. 5 and the Examples. The cargo protein may also be used for treatment and/or prophylaxis of a condition wherein the cargo protein provides a prophylactic and/or therapeutic benefit.

In an embodiment, the disclosure provides a method for modifying one or more primary B cells to provide one or more modified primary B cells. The modified primary B cells maintain allelic exclusion and can participate in a humoral immune response when introduced into a mammal. As noted above, the B cells of this disclosure also co-express any therapeutic protein or other protein of interest, and/or express a functional RNA polynucleotide, and although the remainder of the disclosure describes co-expressed proteins, functional RNAs are also included, non-limiting examples of which include microRNA, shRNA, and any other RNA polynucleotide that can modulate any cellular and/or metabolic process.

In embodiments, a co-expressed protein of this disclosure may be referred to as a “cargo” protein. In embodiments, the co-expressed protein may also be considered a “passenger” protein.

In embodiments, co-expression of the protein is conditional, and can be triggered, for example, by exposing the modified B cells to the antigen to which the heterologous antibody produced by the B cells is specific, e.g., by exposing the B cells to the cognate antigen of the particular antibody expressed by the B cells. Further, elimination of the B cells can be achieved by exposing the B cells to an anti-idiotypic antibody that recognizes an epitope comprised by the variable region of the expressed heterologous antibody, e.g., the antibody that is used to eliminate the described B cells recognizes an idiotype of the expressed antibody.

In more detail, the present disclosure includes, among other features, a novel approach to making and using modified B cells that are configured for conditional expression of any particular protein, in conjunction with expression of least one antibody, or only one antibody that is specific for a defined antigen. Thus, the disclosure provides for what is considered an “on-switch” and an “off-switch.”

In embodiments, the co-expressed protein is engineered for expression in any B cell, including, for example, B cells that are isolated from an individual. In embodiments, the B cells are naïve with respect to the cognate antigen that is recognized by the antibody expressed by the B cells. In an embodiment, the B cells used in the compositions and method of this disclosure are human B cells. In embodiments, the B cells are non-human mammalian cells, and can thus be used for research, and veterinary purposes.

The present disclosure provides, in one embodiment, a method to produce transgenic antibodies in primary B cells using CRISPR-based systems. This method involves in part short term culture in vitro, silencing of the endogenous Ig genes, and insertion of a bi-cistronic or polycistronic cDNA into the Igh locus, and may further include inserting a protein coding sequence into the IgK locus.

As will be described above and further below, the disclosure includes introducing CRISPR-based approaches to modifying B cells. In connection with this, any component used in the described methods can be introduced directly into the B cells as RNA polynucleotides, or they may be transcribed from an introduced DNA template. If tracrRNA is provided as a distinct molecule relative to the crRNA, it may be transcribed from the same intact DNA polynucleotide, or a separate DNA polynucleotide (e.g., one suitable expression vectors). In embodiments, at least two guide RNAs are provided and target opposite strands of one locus, or different loci. Representative examples of guide RNA targeting are shown in the figures. In embodiments, a polynucleotide used in the disclosure, such as a guide RNA, may comprise a backbone modification, e.g., a modification to a sugar or phosphate group in the backbone. In some embodiments, the nucleic acid comprises a nucleobase modification.

In embodiments, a combination of proteins, and a combination one or more proteins and polynucleotides described herein, may be first assembled in vitro and then administered to a cell or an organism. Any protein coding sequence, and any guide RNA sequence or guide RNA coding sequence, can be introduced into B cells using any suitable techniques to modify the B cells such that they express an antibody, and a cargo protein. In embodiments, some or all of the components of the systems used to make the modified B cells are introduced using one or more expression vectors, or by direct introduction of polynucleotides, ribonucleoproteins (RNPs), deoxyribonucleoproteins (DNPs), or a combination thereof.

In general, expression vectors comprise regulatory elements that drive expression of any one or any combination of the described proteins, and/or polynucleotides, and the sequences that encode such proteins and polynucleotides, and may further include any suitable signal sequences. In embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus. In embodiments, a recombinant adeno-associated virus (rAAV) vector may be used. rAAV vectors are commercially available, such as from TAKARA BIO® and other commercial vendors and may be adapted for use with the described systems, given the benefit of the present disclosure. In embodiments, a helper virus may be used to produce rAAV particles. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure. In embodiments, some or all of the components in the described method are delivered using one or more expression vectors. In an embodiment, only the ssDNA template that encodes the cargo protein is introduced to the cells using a viral vector, such as an rAAV vector.

With respect to B cells of the disclosure, mouse and human B lymphocytes typically express a single antibody despite having the potential to express 2 different heavy chains and 4 different light chains. Theoretically the combination could produce 8 different antibodies and a series of additional chimeras that could interfere with the efficiency of humoral immunity and lead to unwanted autoimmunity. Allelic exclusion prevents this from happening and would need to be maintained by any gene replacement strategy used to edit B lymphocytes. In addition, genetic editing is accompanied by safety concerns due to off-target double strand breaks and integrations. In certain embodiments, the currently provided approach lowers these risks by using non-viral gene editing with ssDNA templates, which limits random integrations and by keeping culture time short to prevent expansion of any such cell.

The present approach maintains allelic exclusion in part by ablating the Igkc gene. In the mouse data described below, 95% of B cells express Igkc. In the absence of Igkc expression these cells will die by apoptosis because they cannot survive unless they continue to express a B cell receptor (Kraus et al., 2004; Lam et al., 1997). Since the introduction of the transgene into the heavy chain locus disrupts endogenous Igh expression, editing maintains allelic exclusion in the majority of cells because only cells expressing the introduced antibody can survive. The presently provided strategy also interferes with the survival of cells that suffer off-target integration events, because the majority of such cells would be unable to express the B cell receptor and they too would die by apoptosis.

A potential issue is that there are two heavy chain alleles in every B cell and allelic exclusion would be disrupted if the transgene were only integrated in the non-productive Igh allele allowing for expression of the original productive Igh. However flow cytometry data indicates that this is a very rare event. Thus, either both alleles are targeted or the occasional remaining endogenous Igh gene is unable to pair with the transgenic Igk. A small number of B cells that have not deleted endogenous Igk might also integrate the transgene into the Igh locus. This could decrease the efficiency of knock-in antibody expression if the endogenous kappa pairs with the transgenic heavy chain. The use of a promoterless construct as described below increases surface BCR expression and improves safety. This construct relies on integration into an allele with in frame VDJ rearrangement. Furthermore, the absence of a promoter makes off target gene activation less likely thereby increasing the safety of this approach.

In contrast to the mouse, IGL is expressed by 45% of all B cells in humans. Therefore, this locus would either need to be ablated, or alternatively, cells expressing IGL could be removed from the transferred population by any one of a number of methods of negative selection. The disclosure includes each of these approaches.

Similar to antibody transgenes in mice, expression of the edited BCR varied between different antibodies. Some combinations of heavy and light chains were refractory to expression in mature B cells. In addition, although the level of B cell receptor expression was within the normal range, it was generally in the low end compared to polyclonal B cells. This is consistent with generally lower level expression of a similar transgene in knock-in mice (Jacobsen et al., 2018). Low BCR expression could also be due to the bi-cistronic design since expression was higher in knock-in mice that expressed the identical Ig from the native Igk and Igh loci (Dosenovic et al., 2018). Nevertheless, expression levels were adequate to drive antigen-induced antibody production in vivo.

A non-limiting embodiment of the disclosure is illustrated by engineering mature B cells that express an anti-HIV-1 bNAb. Adoptive transfer of the engineered B cells and immunization with a single cognate antigen led to germinal center formation and antibody production at levels consistent with protection. Consistent with this approach, co-expression of a protein, such as a therapeutic protein, is expected to lead to high levels of the co-expressed protein. While certain co-expressed proteins are illustrated in the Examples and Figures, it will be recognized that the cargo protein is not particularly limited. For example, in embodiments, the cargo protein may be provided as a peptide that can be translated and which may be, for example, from several to 50 amino acids in length, whereas longer sequences are considered proteins. In embodiments, a cargo protein may include template includes a cellular localization signal, or a secretion signal. In embodiments, cargo protein may comprises a transmembrane domain, and thus may be trafficked to, and anchored in a cell membrane, and may further comprise an extracellular domain. In embodiments, the cargo comprise a nuclear localization signal, and thus may be trafficked to and function in the nucleus. In embodiments, the cargo protein comprises one or more glycosylation sites. In embodiments, the cargo protein may encode a binding partner that is distinct from the antibody that the B cells are also engineered to produce. In embodiments, cargo protein may comprise an enzyme; a structural protein; a signaling protein, a regulatory protein; or a storage protein. In embodiments, the cargo protein may comprise a peptide hormone. In embodiments, the cargo protein comprises a protein that is involved in a metabolic pathway. In embodiments, the cargo protein comprises a component of blood. In embodiment, the cargo protein is a therapeutic protein that is intended to treat a disorder of the blood. In an embodiment, the cargo protein is intended to treat or assist with treating an autoimmune disorder, such as an autoimmune disorder wherein B cell function is factor in the disease. In embodiments, the cargo protein influences formation of ectopic germinal centers.

In embodiments, modified B cells of this disclosure are introduced into an individual in need thereof. In embodiments, the individual is in need of the antibody produced by the modified B cells, or is in need of the cargo protein, or is in need of both the antibody and the cargo protein. In embodiments, the individual has been diagnosed with or is suspected of having, or is at risk for contracting an infection, such as an infection by a pathogenic bacteria or a virus. Thus, antibodies produced by the B cell may be specific for an epitope on such a pathogen. Further the cargo protein may also participate in an anti-infection and/or anti-viral response.

The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. In embodiments, 1×10⁴-4×10⁶ modified B cells/kg are administered. In an embodiments, about 3×10⁴-4×10⁵ modified B cells/kg are administered, such as by intravenous administration, but other routes of administration are contemplated. In embodiments, a therapeutically effective amount of B cells is used. In embodiments, a therapeutically effective amount of B cells is an amount of B cells that produces sufficient antibody, or cargo protein, or both, to treat, lessen the severity of, or inhibit the progression of a disease or disorder. The type of antibody and cargo protein are not particularly limited, and representative antibodies and cargo proteins expressed by the modified B cells are described below, and illustrated in the figures, as are methods of making the modified B cells. In embodiments, the cargo comprises coagulation factor VIII or IX to treat hemophilia A or B respectively.

In embodiments, such for research purposes, the cargo protein may be a detectable marker, such as a fluorescent protein, such as green fluorescent protein (GFP), enhanced GFP (eGFP), mCherry, and the like.

The modified primary B cells can also form germinal centers in the individual into which they are introduced. Upon exposure to the cognate antigen, the modified primary B cells produce heterologous antibodies that bind with specificity to a distinct epitope on the cognate antigen. “Heterologous” means the modified B cells produce antibodies that are encoded by the constructs described herein and which are introduced into the modified B cells. Thus, in embodiments, the antibodies are not encoded by the primary B cells before being modified as set forth in this disclosure.

Primary B cells are B lymphocytes that are characterized by having developed in in vivo. In embodiments, primary mature naïve B cells derived from blood or spleen are used. In embodiments, the primary B cells may be memory B cells. In embodiments, the primary B cells used in the methods of this disclosure are IgM or IgD and do not detectably express activation markers at the time they are modified. Memory B cells can express IgM or IgD, or any of switched isotypes. In human samples, CD27 may be used to indicate memory B cells.

The antibodies produced by the modified B cells can contain any suitable framework and hypervariable regions. Any desired complementarity determining regions (CDRs) can be part of the antibodies produced by the modified B cells. Aspects of the disclosure are illustrated with modified B cells that produce IgG and/or IgM antibodies, but the method can be adapted, given the benefit of this specification, to produce any isotope (e.g., any of IgA, IgD, IgE, IgM, and IgG).

The epitope to which the antibodies produced by modified B cells bind is not particularly limited, other than that it will be known so that it can be used to activate the B cells, which results in production of the antibody and the co-expressed protein.

In embodiments, the epitope (and thus the CDRs of the antibodies produced by the modified B cells that confer specificity to the epitope) is present on any protein or other immunogenic substance that can be used to activate the B cells, and for example, coax them to differentiate into plasma cells.

In non-limiting embodiments, B cells are modified according to the constructs and process as generally depicted in FIG. 5. In embodiments, a DNA segment encoding the co-expressed protein is inserted into the IgH or IgK locus of the B cell, which has also been or will be modified to also express a the heavy and light chains of a predetermined antibody. Concurrent modification of one or more loci to facilitate expression of the antibody and the co-expressed protein is an aspect of this disclosure, although in certain embodiments the modification steps may be also performed sequentially.

In certain embodiments, insertion of a DNA segment encoding the co-expressed protein ablates the IgK locus. Further, the disclosure comprises, deletion, disruption, and/or substitution of both the κ light chain and λ light chains coding regions. In embodiments, the disclosure includes insertion of a DNA segment encoding a protein for co-expression with the antibody by multiplexed insertion of the DNA segment encoding said protein into the IgH locus, with concomitant ablation of the IgK locus. In embodiments, the Cₖ exon is used as a target to insert a DNA segment that encodes the co-expressed protein, thereby ablating expression of the endogenously encoded κ light chain. In embodiments, the co-expressed protein, the light chain and the heavy chain of the antibody are expressed from a poly-cistronic message, which may comprise a bi-cistronic message or bi-cistronic segment of a message. Thus, in embodiments, the antibody and the co-expressed protein are encoded by a segment of DNA that includes a polycistronic element and is located between last J gene and the enhancer element, as illustrated by FIG. 5A. In another embodiment, the sequence of the co-expressed protein is introduced into the IgK locus, as further described below.

In embodiments, a CRISPR system is used to initially introduce a ssDNA homology directed repair template (HDRT) into primary B cells. Insertion of the HDRT may be heterozygous or homozygous for any particular allele. The ssHDRT is inserted by function of the CRISPR system that is also introduced into the cell. In embodiments, a method of the disclosure produces a higher frequency of homozygous or heterozygous clones, relative to a control. In embodiments, a method of this disclosure results in higher protein production by the modified B cells, relative to a control. In embodiments, a method of the disclosure results in more antibody production than a control. In embodiments, a method of the disclosure results in more production of a protein that is co-expressed with the antibody, than a control.

The HDRT comprises combinations or sub-combinations the elements described below. In embodiments, the components may be introduced into, for example, the IgH locus using a suitable CRISPR system and guide RNA which facilitates a double stranded break within the IgH locus, and repair of the break by insertion of the HDRT in the IgH locus. In this configuration, the IgK locus is deleted or disrupted using, for example, the same CRISPR system with a suitable guide RNA targeted to the IgK locus, as illustrated in FIG. 5A.

In more detail, and with respect to FIG. 5A, the HDRT is designed such that expression of both the antibody and the co-expressed protein is achieved using a polycistronic insert. The described approach therefore results in a modified B cell comprising in the IgH locus a contiguous DNA sequence encoding a heterologous antibody, the heterologous antibody comprising a variable light chain region, a light chain constant region, and a variable heavy chain region. The same contiguous segment also comprises a sequence encoding a cargo protein that is co-expressed with the heterologous antibody from a polycistronic element. The same contiguous sequence also encodes a variable light chain region, a light chain constant region and a variable heavy chain region. In these and other B cells modified as described herein, may also have the λ light chain locus deleted or disrupted, and the IgK locus in the may also be separately disrupted or deleted. The described contiguous DNA segment as illustrated in FIG. 5A thus comprises, in a 5′ to 3′ orientation, and which may be introduced between a J_{H} and an E_{μ} enhancer segment:

a) a first homology arm used for homologous recombination into the IgH locus;

b) a splice acceptor;

c) a first ribosome skipping sequence;

d) a sequence encoding the cargo protein;

e) a second ribosome skipping sequence;

f) the variable light chain region;

g) a variable light chain constant region;

h) a third ribosome skipping sequence;

i) the heavy chain variable sequence;

j) a splice donor; and

k) a second homology arm used for recombination in to the IgH locus.

In a related approach, the construct described above is used for insertion into the IgH locus in the same manner, with the exception that this construct does not contain the sequence encoding the protein for co-expression with the antibody. Thus, instead of separately targeting the IgK locus solely by the operation of the CRISPR system, the IgK locus is instead modified by the CRISPR system to encode the co-expressed protein. This is illustrated in FIG. 5B, depicting the sequence encoding the protein for co-expression as flanked with homology arms targeted to the IgK locus. This targeting process may include use of sequences directed to the Cκ exon, and other features such as the P2A site, and a stop codon, as illustrated in FIG. 5B.

In more detail, as depicted in FIG. 5B, the disclosure provides modified B cells that comprise a first sequence encoding a heterologous antibody, the heterologous antibody comprising a variable light chain region, a light chain constant region and a variable heavy chain region. The first sequence is introduced into the IgH locus. However, a second sequence encoding a cargo protein is introduced into the IgK locus, and can be introduced into the Cₖ exon of the IgK locus. In this case, the λ light chain locus may also disrupted or deleted. In this approach, two ssDNA repair plates are used, one for the IgH locus, and one for the IgK locus. Thus, in this approach, the modified B cell comprises:

a) a first homology arm used for homologous recombination into the IgH locus;

b) a splice acceptor;

c) a first ribosome skipping sequence;

d) the variable light chain region;

e) a variable light chain constant region;

f) a second ribosome skipping sequence;

g) the heavy chain variable sequence;

h) a splice donor; and

i) a second homology arm used for recombination in to the IgH locus;

and wherein the second sequence encoding the cargo protein is homologously recombined into the IgK locus and comprises in a 5′ to 3′ orientation:

j) a third homology arm used for recombination into the IgK locus;

k) a first C_{κ} segment, which may be all or a part of the homology arm which places a 3′ sequence within a C_{κ} exon;

l) a ribosome skipping sequence;

m) a sequence encoding the cargo protein;

n) a stop codon;

o) a second C_{κ} segment; and

p) a fourth homology arm used for recombination into the IgK locus.

Other features may be included in the constructs used to make the modified B cells of this disclosure, such as nucleotides from constant mu (Cμ) exon 1, amino acid linker sequence(s), a sequence encoding a kappa constant region (Cκ), as generally described herein.

In a third embodiment, as depicted in FIG. 5C, the antibody components may be provided on separate HDRTs, one of which also encodes the cargo protein. In this approach, a first HDRT comprises a first homology arm that is homologous to the IgH locus, an SV40 polyadenylation signal or similar sequence is used to stop translation from the 5′ endogenous Vh promoter of the endogenously rearranged variable region, a V_{H} promoter, a heavy chain LVDJ sequence, a J_{H}H splice donor, and a second homology arm that is homologous to a segment of the IgH locus. This can be introduced into a segment of the IgH locus that is between a J_{H} and an Eμ enhancer segment. In this construct, the LVDJ segment need not be in frame with an endogenous open reading frame, whereas in the examples in FIGS. 5A and 5B, in-frame insertions are made, but in embodiments, the in-frame insertions are only made in the IgK locus. A second HDRT is inserted in the IgK locus, which may be downstream (e.g., 3′) of a Jk5 segment, and before an iEκ enhancer segment. The second HDRT comprising a first homology arm that is homologous to the IgK locus, a SV40 polyadenylation signal and Vic promoter, the sequence encoding the cargo protein, a P2A or similar site, a light chain LVJ segment, a Jκ splice donor, and a second homology arm that is homologous to a segment of the IgK locus.

In embodiments, a splice acceptor used in the compositions and methods of this disclosure may comprise an AG nucleotide sequence, and may further comprise a branch sequence. In embodiments, nucleotides from constant mu (Cμ) exon 1 are from any suitable such exon sequence, so as to facilitate disruption of the κ light chain. In embodiments, the nucleotides are inserted such that they maintain the downstream reading frame of the remainder of the construct, and any number of nucleotides can be used. Since the first codon in the exon is split between the J and the constant region with the first nucleotide encoded by J and the other two nucleotides by the C region, the following equation applies for the number of nucleotides (nucleotides ×3)-1. The sequence of the constant mu (Cμ) exon 1 is known and can be accessed at, for example, NCBI Gene ID 3507, Ensembl ENSG00000211899.

The first amino acid linker is typically three amino acids long, and may be comprised of a GSG sequence. A self-cleaving amino acid sequence is typically about 18-22 amino acids long. Any suitable sequence can be used, non-limiting example of which include T2A, comprising the amino acid sequence: EGRGSLLTCGDVEENPGP (SEQ ID NO:1); P2A, comprising the amino acid sequence ATNFSLLKQAGDVEENPGP (SEQ ID NO:2); E2A, comprising the amino acid sequence QCTNYALLKLAGDVESNPGP (SEQ ID NO:3); and F2A, comprising the amino acid sequence VKQTLNFDLLKLAGDVESNPGP (SEQ ID NO:4).

The kappa constant region is known, and can be accessed at, for example NCBI Gene ID 3514, Ensembl ENSG00000211592. Alternatively, a lambda constant region may be used, and its sequence is also know. The sequence of the protease-cleavage site is typically about 4 amino acids long. In a non-limiting embodiment, the sequence is RRKR (SEQ ID NO:55). The sequence of the second amino acid linker sequence may be the same as the first amino acid linker. The intron splice donor site can be of variable length, typically about 40-60 nucleotides. In embodiments, the intron splice donor comprises a GU sequence.

The first and second homology arms are configured to be introduced into one or more desired chromosomal loci, as illustrated herein, and in particular by FIG. 5. In embodiments, the disclosure comprises combined endogenous Ig disruption with insertion of a transcription unit (the HDRT) that directs expression of the heavy and light chain into an endogenous heavy chain locus/loci, along with a suitable DNA segment that encodes the co-expressed protein. In embodiments, such loci comprise the IGKC exon, an IGHJ6 intron, an IgLC locus, or a combination thereof. The sequence of the IGKC exon can be accessed at, for example, NCBI Gene ID 3514, Ensembl ENSG00000211592). The sequence of IGHJ6 introns can be accessed at, for example, NCBI Gene ID 28475, Ensembl ENSG00000211900). There are five functional genes in the IgLC locus, and any can be used for the homology arm. The sequence of the five genes in the IgLC locus are IGLC1, IGLC2, IGLC3, IGLC6 IGLC7, and can be accessed at, for example, NCBI Gene ID 3537, 3538, 3539 3542, 28834, respectively, Ensembl ENSG00000211675, ENSG00000211677, ENSG00000211679, ENSG00000222037, ENSG00000211685, respectively. Thus, the sequences of the first and second homology arms may be identical to the chromosomal sequences into which they are introduced and/or replace. Non-limiting examples of HDRT sequences used in this disclosure are provided in Table 2 and FIG. 13. In embodiments, the homology arms are from 60 nucleotides to about 3 kb in length. The sequences provided are representative DNA coding sequences, but can be changed based on, for example, alternative codons for the encoded proteins. In embodiments, a construct of the disclosure is codon optimized for expression in, for example, human cells.

In addition to the HDRT, the disclosure comprises introducing into primary B cells a clustered regularly interspaced short palindromic repeats (CRISPR)-Cas (CRISPR-associated proteins) system. The disclosure is illustrated using a Cas9 enzyme, but it is expected that other CRISPR systems and Cas enzymes can be used. In embodiments, any type II CRISPR system/Cas enzyme is used. In embodiments, the type II system/Cas enzyme is type II-B. In embodiments, that Cas enzyme comprises Cpf1.

In embodiments, the disclosure includes introducing the HDRT, the Cas enzyme, a trans-activating RNA (tracrRNA), and one, two, or three guide RNAs. Suitable tracrRNAs are known in the art and can be adapted for use with the methods of this disclosure. The guide RNAs may be provided as crRNAs or sgRNAs without tracrRNA. The guide RNAs are programmed to target specific sites so that the first and second homology arms are integrated correctly, depending on the locus where the HDRT is inserted. In embodiments, two or three guide RNAs are used. In embodiments, the guide RNAs are targeted to a suitable sequence in the IGKC, IGHJ6, and/or IgLC loci. In embodiments, at least one guide RNA is targeted to the Cκ exon in the IgK locus.

Methods for designing suitable guide RNAs are known in the art such that guide RNAs having the proper sequences can be designed and used, when given the benefit of the present disclosure. Non-limiting examples of guide RNAs are provided in Table 1, wherein the DNA sequence corresponding to the guide RNA sequences is provided. e.g., each T is replaced by a U.

In embodiments, insertion of an HDRT described herein into a plurality of primary B cells results in more of the primary B cells being λ-B cell receptor positive primary B cells than κ-B cell receptor positive primary B cells. In embodiments, insertion of an HDRT as describe herein reduces or eliminates λ-B cell receptor positive primary B cells, and/or reduces or eliminates κ-B cell receptor positive primary B cells.

In embodiments, an HDRT of this disclosure comprises at least one of the following characteristics:

i) no promoter is included in the HDRT;

ii) the primary B cells are human B cells;

iii) only two nucleotides from the C_{μ} exon 1 are included in the HDRT;

iv) the first or second self-cleaving amino acid sequences comprise a T2A sequence or a P2A sequence;

v) a first or second amino acid linker sequence, or both, are GSG-linker sequences, and may be positioned before P2A or T2A sequences;

vi) a protease cleavage site is a furin-cleavage site, which may be positioned after the light chain constant region before the GSG-P2A;

vii) the CAS enzyme and the guide RNAs are introduced into the primary B cell as a ribonucleotide protein complex;

viii) production of the modified primary B cells is performed without using a retroviral delivery vector.

In embodiments, the disclosure comprises providing a treatment to an individual in need thereof by introducing a therapeutically effective amount of modified B cells as described herein to the individual, and vaccinating the individual with an antigen that is cognate to the antibodies produced by the modified B cells, to trigger expression of the antibody and the co-expressed protein. Thus, the antigen used in the vaccination comprises an epitope that is specifically recognized by the antibodies produced by the modified B cells. One or more vaccinations can be used.

In embodiments, the disclosure provides for reducing the amount of modified B cells in an individual, to thereby obviate expression of the particular antibody expressed by the B cells, and also, importantly, to reduce and/or eliminate expression of the co-expressed protein. In this regard, vaccination with the cognate antigen renders the B cells vulnerable to targeting due to expression of the particular heterologous antibody that they have been engineered to express, as described herein. Accordingly, the B cells can be targeted and eliminated using any agent that binds with specificity to the idiotype comprised by the variable regions of the described antibody. Thus, in one embodiment, anti-idiotypic antibodies are administered to the individual in an amount sufficient to reduce and/or eradicate the modified B cells from the individual. In one non-limiting embodiment, the anti-idiotypic antibody functions as an antigen blocking agent, and therefore binds to the paratope of antibody, but other binding parameters are included within the scope of this disclosure.

In embodiments, the disclosure includes modified B cells made according to a method of this disclosure. In embodiments, the modified B cells can be provided in a pharmaceutical formulation, and such formulations are included in the disclosure. A pharmaceutical formulation can be prepared by mixing the modified B cells with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, Pa. Lippincott Williams & Wilkins, the disclosure of which is incorporated herein by reference.

In embodiments, the disclosure includes obtaining B cells or B cell progenitors from an individual, modifying the cells ex vivo using a system as described herein, and reintroducing the cells or their progeny into the individual or an immunologically matched individual for prophylaxis and/or therapy of a condition, disease or disorder.

In embodiments, the cargo protein may be provided as a peptide that can be translated and which may be, for example, from several to 50 amino acids in length, whereas longer sequences are considered proteins. In embodiments, a cargo protein may include template includes a cellular localization signal, or a secretion signal. In embodiments, cargo protein may comprises a transmembrane domain, and thus may be trafficked to, and anchored in a cell membrane, and may further comprise an extracellular domain. In embodiments, the cargo comprise a nuclear localization signal, and thus may be trafficked to and function in the nucleus. In embodiments, the cargo protein comprises one or more glycosylation sites. In embodiments, the cargo protein may encode a binding partner that is distinct from the antibody that the B cells are also engineered to produce. In embodiments, cargo protein may comprise an enzyme; a structural protein; a signaling protein, a regulatory protein; or a storage protein. In embodiments, the cargo protein may comprise a peptide hormone. In embodiments, the cargo protein comprises a protein that is involved in a metabolic pathway. In embodiments, the cargo protein comprises a component of blood. In embodiment, the cargo protein is a therapeutic protein that is intended to treat a disorder of the blood. In embodiments, the cargo protein is a secreted protein. In an embodiment, the cargo protein is intended to treat or assist with treating an autoimmune disorder, such as an autoimmune disorder wherein B cell function is factor in the disease. In embodiments, the cargo protein influences formation of ectopic germinal centers.

In embodiments, modified B cells of this disclosure are introduced into an individual in need thereof. In embodiments, the individual is in need of the antibody produced by the modified B cells, or is in need of the cargo protein, or is in need of both the antibody and the cargo protein. In embodiments, the individual has been diagnosed with or is suspected of having, or is at risk for contracting an infection, such as an infection by a pathogenic bacteria or a virus. Thus, antibodies produced by the B cell may be specific for an epitope on such a pathogen. Further, the cargo protein may also participate in an anti-infection and/or anti-viral response.

In embodiments, the disclosure comprises a kit for use in making modified B cells. In embodiments, the kit can comprising one or more cloning vectors, the vectors comprising the elements discussed above for producing an HDRT, with the exception that the vector contains suitable restriction enzyme recognition sites for inserting a sequence encoding the VJ regions of the heterologous antibody light chain, and for inserting a sequence encoding the VDJ regions of the heterologous heavy antibody chain. Guide RNAs and a Cas enzyme may also be provided with the kit. The kit may also include suitable reagents for insertion of a DNA segment that encodes the co-expressed protein in the IgH locus in a cassette with the antibody coding segments, or for insertion of a DNA segment that encodes the co-expressed protein into the IgK locus.

In embodiments, the disclosure comprises an isolated HDRT. Methods for producing ssDNA homology directed repair templates are known and can be adapted for use with the present disclosure. In a non-limiting embodiment, plasmids comprising the HDRT are digested with sequence-specific nickases, and ssDNA purification is performed using any suitable technique, such as by agarose gel electrophoresis. In embodiments, the ssDNA repair template is designed to replace an open reading frame. In embodiments, the ssDNA repair template comprises a modified open reading frame. In embodiments, expression of coding sequences comprised by the ssDNA templates is controlled by an endogenous promoter. An “endogenous” promoter is a promoter that is operatively linked to the gene into which the ssDNA sequence is introduced (and subsequently rendered double stranded by the cell) and was present in said operative linkage with the gene prior to insertion of the ssDNA templates. Thus, in embodiments, the ssDNA templates may be free of any promoter that is operably linked to an open reading frame, and wherein said promoter is operable in the cell into which a repair template introduced

In embodiments, the disclosure comprises isolating a sample from a mammal, identifying antibody coding sequences from the sample, and engineering B cells to express the identified antibody sequences.

In embodiments, the disclosure comprises obtaining a sample comprising B cells from an individual, determining the sequence of the VJ regions of an antibody light chain, determining the sequence of the VDJ chain of the same antibody, and generating an HDRT comprising the sequences encoding the VJ and VDJ regions. The disclosure further includes using the HDRT to produce modified B cells that comprise the VJ and VDJ regions, and which produce the antibodies, and further comprises modifications for co-expressing a cargo protein, as described herein.

The following examples are meant to illustrate, and are not intended to be limiting. The disclosure includes all reagents and process steps that are described in these examples.

### Example 1

Expressing Antibodies in Primary Mature, Murine B Cells.

To edit mature B cells efficiently, the disclosure comprise activating and culturing the B cells in vitro. To determine whether such cells can participate in humoral immune responses in vivo we used Igh^{a} CD45.1 B cells carrying the B1-8^{hi} heavy chain that are specific for the hapten 4-hydroxy-3-nitro-phenylacetyl (NP) (Shih et al., 2002). B1-8^{hi} B cells were activated in vitro with anti-RP105 antibody for 1-2 days and subsequently transferred into congenically marked (Igh^{b} CD45.2) C57BL/6J mice. Recipients immunized with NP-conjugated to ovalbumin (NP-OVA) developed germinal centers (GCs) containing large numbers of the antigen-specific, transferred B cells (FIG. 8A, B) and produced high levels of antigen-specific IgG1 (FIG. 8 C). In addition, transfection by electroporation did not affect the ability of transferred cells to enter GCs (FIG. 8 D, E).

Despite having two alleles for each of the antibody chains, B cells express only one heavy and one light chain gene, a phenomenon referred to as allelic exclusion (Cebra et al., 1966; Nussenzweig et al., 1987; Pernis et al., 1965). In the absence of the present disclosure, introducing additional antibody genes would risk random combinations of heavy and light chains some of which could be self-reactive or incompatible. Thus, deletion of the endogenous chains would be desirable to prevent expression of chimeric B cell receptors (BCRs) composed of the transgene and the endogenous antibody genes. To do so, we combined endogenous Ig disruption with insertion of a transcription unit that directs expression of the heavy and light chain into the endogenous heavy chain locus.

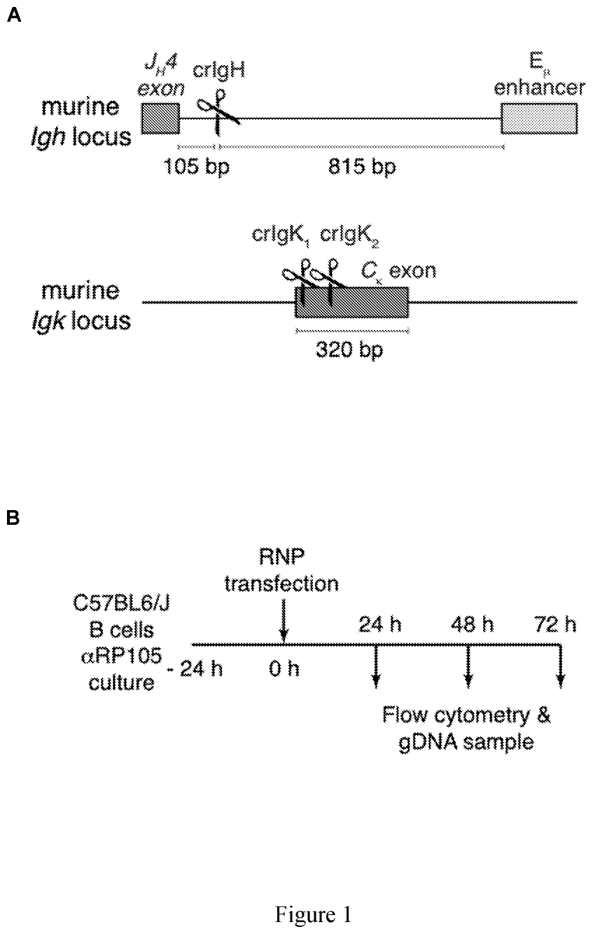

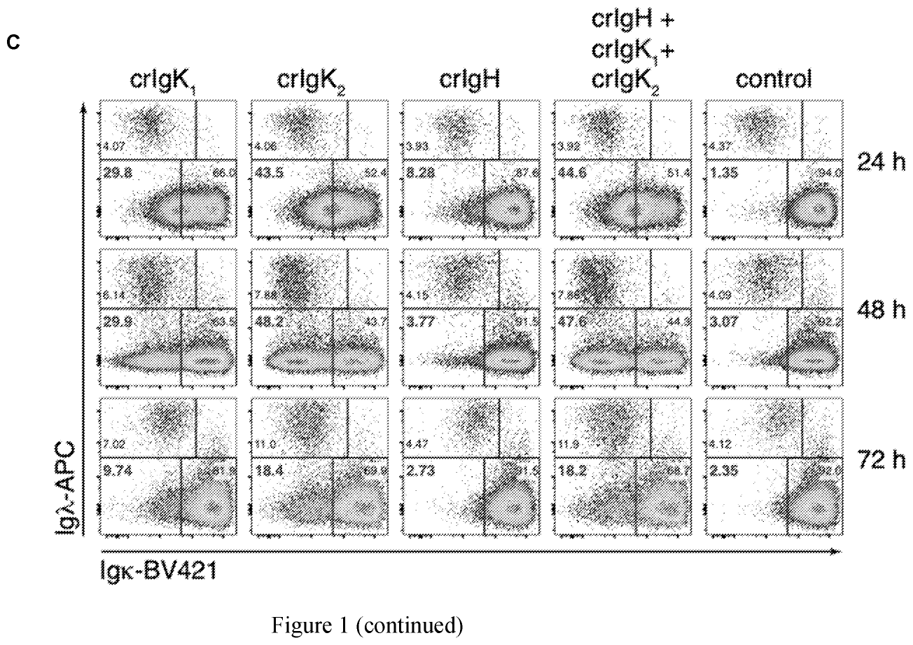

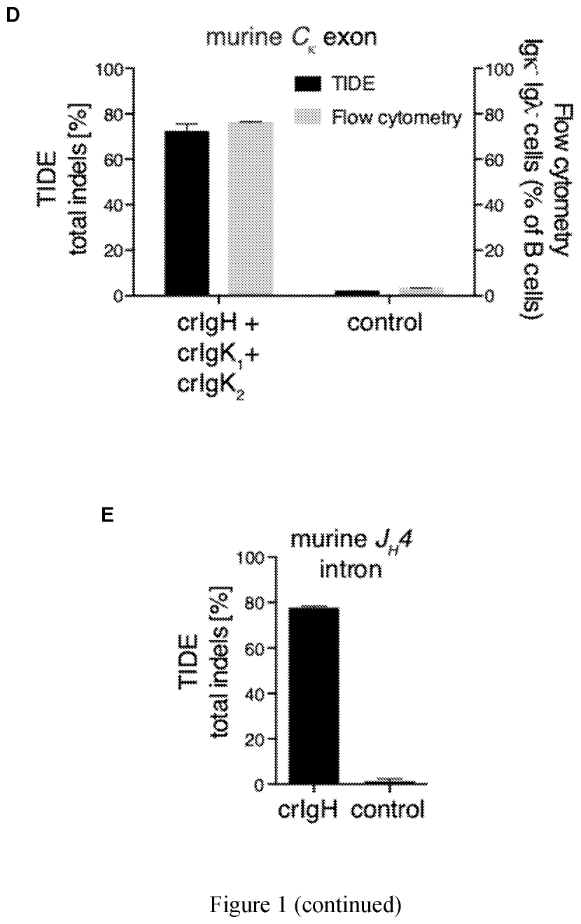

Guide RNAs (crRNAs or sgRNAs) were designed to ablate the κ-light chain because 95% of all mouse B cells express Igk (FIG. 1A). The efficiency of κ light chain deletion was measured by flow cytometry using the ratio of κ/λ, cells to normalize for cell death due to BCR loss. The selected crRNAs consistently ablated Igκ expression by 70-80% of B cells as measured by flow cytometry or TIDE (Tracking the Indels by DEcomposition (Brinkman et al., 2014)) analysis (FIG. 1 B-D).

To insert a transgene into the heavy chain locus we designed guide RNAs specific for the first Igh intron immediately 3′ of the endogenous VDJ_{H} gene segment, and 5′ of the Eμ enhancer. The Eu enhancer sequence is known in the art, and is located in the IGHJ6 intron, the sequence of which is described above. This position was selected to favor transgene expression and allow simultaneous disruption the endogenous heavy chain (see below and (Jacobsen et al., 2018, the disclosure of which is incorporated herein by reference)). We tested 7 crRNAs and selected a high-efficiency crRNA located 110 bp downstream of the J_{H}4 intron producing 77% indels by the TIDE assay (FIG. 1 E, FIG. 9A, B). This location also allowed for sufficient homology to introduce a transgene, irrespective of the upstream VDJ rearrangement.

The homology-directed repair template is described above. In an embodiment, it is composed of a splice acceptor (SA) stop cassette to terminate transcription of upstream rearranged VDJ_{H}, and a V_{H}-gene promoter followed by cDNAs encoding Igk, a P2A self-cleaving sequence, and IgV_{H} with a J_{H}1 splice donor (SD) site (FIG. 2A). This design disrupts expression of the endogenous locus, while encoding a transcription unit directing expression of the introduced heavy and light chain under control of endogenous Igh gene regulatory elements. In addition, it preserves splicing of the transgenic IgV_{H} into the endogenous constant regions allowing for expression of membrane and secreted forms of the antibody as wells as different isotypes by class switch recombination. Finally, correctly targeted cells are readily identified and enumerated by flow cytometry because they bind to cognate antigen.

A number of methods for producing ssDNA homology directed repair templates (HDRTs) were compared. The most reproducible and least cytotoxic involved digestion of plasmids with sequence-specific nickases, and ssDNA purification by agarose gel electrophoresis (FIG. 9 C-E) (Roth et al., 2018; Yoshimi et al., 2016).

Co-transfection of the ssDNA template with pre-assembled Cas9 ribonucleoproteins (RNPs) containing the crRNAs resulted in expression of the encoded anti-HIV antibody in 0.1-0.4% of mouse B cells by antigen-specific flow cytometry using antigens TM4 core (McGuire et al., 2014; McGuire et al., 2016) or 10mut (Steichen et al., 2016) (FIG. 2 C,D, FIG. 10A). Transgene expression was stable over the entire culture period of 3 days on feeder cells (Kuraoka et al., 2016), during which the overall number of B cells expanded by 6 to 20-fold (FIG. 2 E-H). However, expression of transgenic antibodies differed depending on the antibody and were generally reflective of their expression in knock-in mouse models (FIG. 2 C, F) (Dosenovic et al., 2018; Dosenovic et al., 2015; Escolano et al., 2016; McGuire et al., 2016; Steichen et al., 2016).

To determine whether edited cells are allelically excluded at the heavy chain locus we transfected Igh^{a/b} cells with 3BNC60^{SI}, a chimeric antibody composed of the mature heavy chain and germline light chain of the anti-HIV bNAb 3BNC60 (FIG. 10 B, C). The majority of edited cells expressing the 3BNC60^{SI} transgene, expressed it using either Igm^{a} or Igm^{b} allele as determined by flow cytometry. Only 5.21% of 3BNC60^{SI}-expressing B cells showed co-expression of both IgM^{a} and IgM^{b} indicative of allelic inclusion of the endogenous allele or successful integration of the transgene into both alleles. Thus, the majority of edited B cells express only the transgene.

Promoter containing expression cassettes have the potential to cause unwanted ectopic gene expression or allelic inclusion since they can be expressed from either the rearranged or germline IgH locus. To address these potential problems we designed a smaller, promoterless antibody expression cassette that depends on integration into a rearranged IgH allele for expression (FIG. 10 D). Cell surface expression of the 3BNC60^{SI} from the promoterless construct was higher than the promoter-driven version (FIG. 10 E, F). Thus, the smaller promoterless, and potentially safer construct efficiently directs knock-in antibody expression.

Without intending to be bound by any particular theory, we conclude that mature mouse B cells can be edited in vitro to produce anti-HIV-1 bNAbs from the Igh locus.

### Example 2

Antibody Gene Editing in Human B Cells

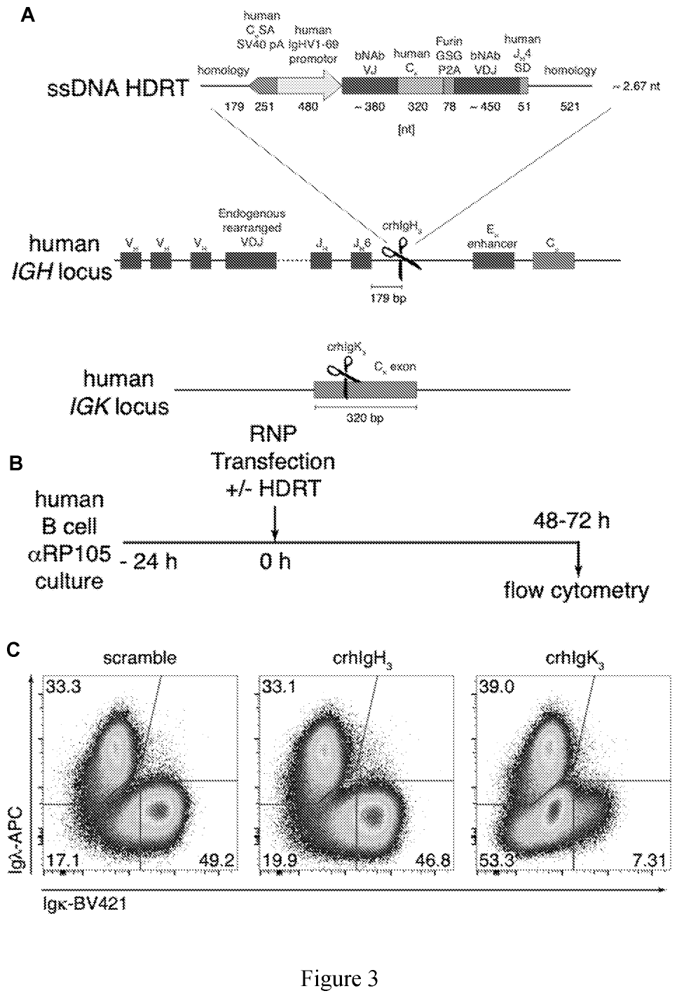

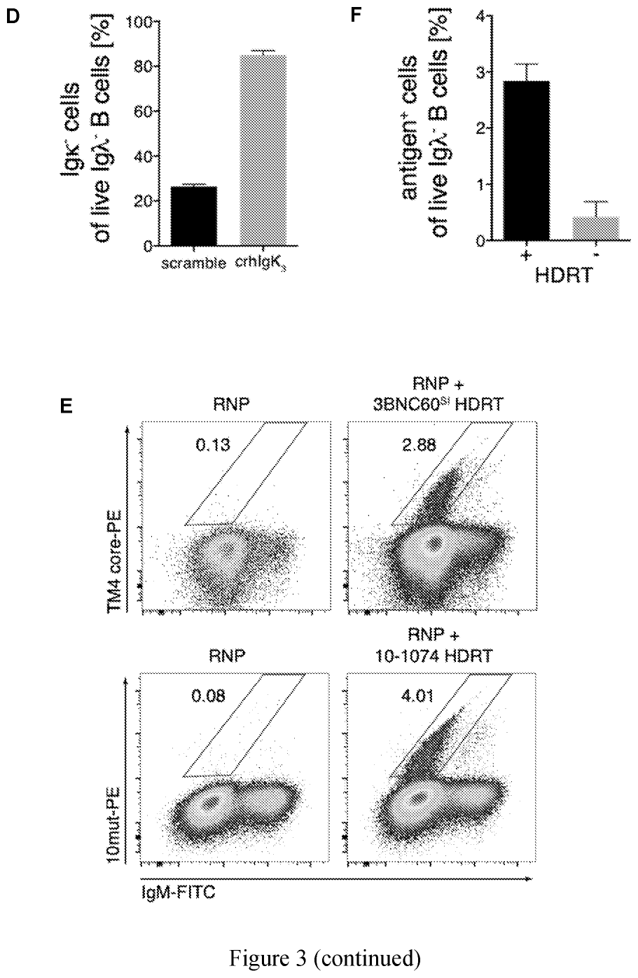

To determine whether this method could be adapted to edit human B cells we isolated them from peripheral blood of healthy volunteers and activated them using an anti-human RP105 antibody (Miura et al., 1998). Analogous crRNAs were selected for targeting the human IGKC and the first intron 3′ of IGHJ6 (FIG. 3A-D, FIG. 11A, B). The best IGKC-targeting crRNA caused 85% of κ-bearing B cells to lose BCR expression, whereas λ-bearing cells increased proportionally indicating that they were unaffected. TIDE analysis of the J_{H}6 intron sequences showed that the most efficient crRNA induced 64% indels. In conclusion, activation of human, primary B cells with anti-RP105 allows efficient generation of indels using Cas9 RNPs.

To target bNAbs into the human J_{H}6 intron we adapted the ssDNA HDRT and replaced mouse with human homology arms, the human Cμ splice acceptor, the human IGHV1-69 promoter, a codon-modified human IGKC constant region to avoid targeting by crRNAs and the human J_{H}4 splice donor (FIG. 3A). In contrast to mouse cells, 2.9-4% of B cells expressed 3BNC60^{SI} or 10-1074 antibodies respectively as determined by flow cytometry using the cognate antigen (FIG. 3 E, F). Thus, the efficiency of transgene integration is at least 10-times higher in human B cells. Furthermore, viability was also higher in human B cells, ranging from 60 to 85% of live cells after transfection (FIG. 11 C).

Without intending to be bound by any particular theory, we conclude that primary human B cells can be edited by CRISPR/Cas9 to express anti-HIV bNAbs, and that this is significantly more efficient than in mouse B cells.

### Example 3

Adoptive Transfer of Antibody-Edited B Cells

To determine whether edited B cells can participate in immune responses, we adoptively transferred mouse 3BNC60^{SI}-edited Igh^{b} B cells, into congenically-marked Igh^{a} wild type mice and immunized with the high-affinity, cognate antigen TM4 core in Ribi adjuvant (FIG. 4A). Transgene-specific responses were detected using anti-idiotypic antibodies as an initial capture reagent in ELISA. Similar to endogenous humoral immune responses, transgenic antibodies were detected on day 7 after immunization, they peaked at day 14 and started to decrease by day 21 (FIG. 4 B, C). Importantly, the transgenic immune response included secondary isotypes indicating that the re-engineered locus supports class switch recombination (FIG. 4 C). Finally, the magnitude of the response was directly correlated to the number of transferred cells. However, prolonged in vitro culture under the conditions tested decreased the efficiency of antibody production in vivo (FIG. 4 D).

To determine whether the transferred cells retained the ability to produce neutralizing antibodies we used B cells that were edited to produce 10-1074, a potent bNAb, or 3BNC60^{SI} a chimeric antibody with limited neutralizing activity (Dosenovic et al., 2018; Mouquet et al., 2012). 4×10⁷ transfected B cells were transferred into wild type Igh^{a} mice that were subsequently immunized with the appropriate cognate antigen 10mut (Steichen et al., 2016) or TM4 core (Dosenovic et al., 2018; Dosenovic et al., 2015; McGuire et al., 2014; McGuire et al., 2016). IgG was purified from the serum of 3 mice that received an estimated ˜103 edited B cells expressing 10-1074 or 3BNC60^{SI}. The purified serum antibodies were tested for neutralizing activity in the TZM-bl assay (Montefiori, 2005). Two of the 3 mice that received 10-1074 edited cells showed IC50s of 21.59 μg/mL and a third reached 49% neutralization at 118 μg/mL (corresponding to approximately 1:500 and 1:100 dilution of serum, FIG. 4 E, FIG. 12A, B). As expected, neutralizing activity was not detected in mice receiving 3BNC60^{SI} because this antibody is 2-3 orders of magnitude less potent against the tested viral strains than 10-1074 (FIG. 12 C).

Without intending to be bound by theory, we conclude that edited B cells can be recruited into immune responses and produce sufficient antibody to confer potentially protective levels of humoral immunity (Shingai et al., 2014).

### Example 4

This Example provides a description of the materials and methods used to produce the foregoing results.

Guide RNA Design

Guide RNAs were designed with the MIT guide design tool (crispr.mit.edu), CHOPCHOP (chopchop.cbu.uib.no) and the IDT crRNA design tool (www.idtdna.com). Designs were synthesized by IDT as Alt-R CRISPR-Cas9 crRNAs or sgRNAs. Guide RNA sequences are listed in Table 1. The guide RNAs are related to FIG. 9A.

TABLE 1
Guide RNA sequences

| | crRNA | | |
|---|---|---|---|
| | sequence | SEQ | |
| | without | ID | |
| Name | PAM | NO: | Locus |
| crIgK₁ | GTTCAAGAAGCACACGACTG | 5 | mouse Igkc |
| crIgK₂ | GTTAACTGCTCACTGGATGG | 6 | mouse Igkc |
| crIgH | GGAGCCGGCTGAGAGAAGTT | 7 | mouse J_{H}4 intron |
| crIgH_B | GTGGAGATAATCTGTCCTAA | 8 | mouse J_{H}4 intron |
| crIgH_C | AGTCCCTATCCCATCATCCA | 9 | mouse J_{H}4 intron |
| crIgH_D | TGAGCATTGCAGACTAATCT | 10 | mouse J_{H}4 intron |
| crIgH_E | AAGTCCCTATCCCATCATCC | 11 | mouse J_{H}4 intron |
| crIgH_F | TCTTGGATATTTGTCCCTGA | 12 | mouse J_{H}4 intron |
| crIgH_G | GTTGGGAAATAAACTGTCTA | 13 | mouse J_{H}4 intron |
| crhIgK₁ | GGTGGATAACGCCCTCCAAT | 14 | human IGKC |
| crhIgK₂ | GTGGATAACGCCCTCCAATC | 15 | human IGKC |
| crhIgK₃ | CTGGGAGTTACCCGATTGGA | 16 | human IGKC |
| crhIgK₄ | CCTCCAATCGGGTAACTCCC | 17 | human IGKC |
| crhIgK₅ | ATCCACCTTCCACTGTACTT | 18 | human IGKC |
| crhIgK₆ | TTCAACTGCTCATCAGATGG | 19 | human IGKC |
| crhIgK₇ | GATTTCAACTGCTCATCAGA | 20 | human IGKC |
| crhIgK₈ | TGGGATAGAAGTTATTCAGC | 21 | human IGKC |
| crhIgK₉ | ATTCAGCAGGCACACAACAG | 22 | human IGKC |
| crhIgK₁₀ | GGCCAAAGTACAGTGGAAGG | 23 | human IGKC |
| crhIgH₁ | GTCCTCGGGGCATGTTCCGA | 24 | human J_{H}6 intron |
| crhIgH₂ | TCCTCGGGGCATGTTCCGAG | 25 | human J_{H}6 intron |
| crhIgH₃ | AGGCATCGGAAAATCCACAG | 26 | human J_{H}6 intron |
| crhIgH₄ | CTCAGGTTGGGTGCGTCTGA | 27 | human J_{H}6 intron |
| crhIgH₅ | ACGAGATGCCTGAACAAACC | 28 | human J_{H}6 intron |
| crhIgH₆ | ACCTGAGTCCCATTTTCCAA | 29 | human J_{H}6 intron |
| crhIgH₇ | TCAGCCATCACTAAGACCCC | 30 | human J_{H}6 intron |
| crhIgH₈ | CAAACCAGGGGTCTTAGTGA | 31 | human J_{H}6 intron |
| crhIgH₉ | CTAAGACCCCTGGTTTGTTC | 32 | human J_{H}6 intron |
| crhIgH₁₀ | TCAGGCATCTCGTCCAAATG | 33 | human J_{H}6 intron |
| Mus_ | AAGACACAGGTTTTCATGTT | 34 | mouse J_{K}5 intron |
| sgIgKJ5₁ | | | |
| Mus_ | GGGCTCATTATCAGTTGACG | 35 | mouse J_{K}5 intron |
| sgIgKJ5₂ | | | |
| Mus_ | GGTCTTCTAGACGTTTAAGT | 36 | mouse J_{K}5 intron |
| sgIgKJ5₃ | | | |

AAV HDRT Preparation

HDRT sequences, listed in Table 2, were synthesized as gBlocks (IDT) and cloned using NheI and XhoI (NEB) into vector pAAV using traditional restriction enzyme cloning or NEB Hifi DNA assembly kit. pAAV was packaged into AAV6 by Vigene Biosciences.

ssDNA HDRT Preparation

HDRT sequences, listed in Table 2, were synthesized as gBlocks (IDT) and cloned using NheI and XhoI (NEB) into vector pLSODN-4D from the long ssDNA preparation kit (BioDynamics Laboratories, Cat. #DS620). ssDNA was prepared following the manufacturer's instructions with the following modifications: In brief, 2.4 mg sequence verified vector was digested at 2 μg/μL in NEB 3.1 buffer with 1200 U Nt.BspQI for 1 h at 50° C. followed by addition of 2400 U XhoI (NEB) and incubation for 1 h at 37° C. Digests were desalted by ethanol precipitation and resuspended in water at <1 μg/μL. An equal volume of formamide gel-loading buffer (95% de-ionized formamide, 0.025% bromophenol blue, 0.025% xylene cyanol, 0.025% SDS, 18 mM EDTA) was added and heated to 70° C. for 5 min to denature dsDNA. Denatured samples were immediately loaded into dye-free 1% agarose gels in TAE and run at 100 V for 3 h. Correctly sized bands were identified by partial post-stain with GelRed (Biotium), then excised and column purified (Machery Nagel Cat. # 740610.20 or 740609.250) according to the manufacturer's instructions. Eluate was ethanol precipitated, resuspended in water, adjusted to 2.5 μg/μL and stored at −20° C.

TABLE 2

```
gBlock sequences of HDRTs
(Table 2 is reproduced as FIG. 13,
  with nucleotidekey included).
3BNC60^{SI}, mouse
GCATAGCTAGCGCTCTTCAGTAAGAATGGCCTCTCCAGGT
CTTTATTTTTAACCTTTGTTATGGAGTTTTCTGAGCATTG
CAGACTAATCTTGGATATTTGTCCCTGAGGGAGCCGGCTG
AGAGAAGTTAAGAGTAGCAACAAGGAAATAGCAGGGTGTA
GAGGGATCTCCTGTCTGACAGGAGGCAAGAAGACAGATTC
TTACCCCTCCATTTCTCTTTTATCCCTCTCTGGTCCTCAG
AGAGTTAGAACTTGTTTATTGCAGCTTATAATGGTTACAA
ATAAAGCAATAGCATCACAAATTTCACAAATAAAGCATTT
TTTTCACTGCATTCTAGTTGTGGTTTGTCCAAACTCATCA
ATGTATCTTATCATGTCTGGTCGACAGTATGCAGAGGGCT
GTATCCACTGGAGAGGATGAAGTCACTGAGTTGGAAAACA
GAACAGGACAGGCACCTAACAAGTGGTTGCTATAGCCCAC
TGTTACCCTTTTACATGTATAGGCTCAGGATAAGCAGTGA
TACTGTGAGGTTTATGTGTGAGAACATCACAGTATAAACA
CATCTCAATAGAGGTCTTAGAGATCAGCACAATTAGTGAG
AAGTCATAAACAGTAGATACTATAAGGCATAGGCTCAGCT
ACCTAGGGTCAGGTATCTGTGTAAATCTGATTGTGTATCA
GGTTTAGATCAATATGACTTAGGGAGGCGAGTCATATGCA
AATCTAAGAAGACTTTAGAGAAGAAATCTGAGGCTCACCT
CACATAACAGCAAGAGAGTGTCCGGTTAGTCTCAAGGAAG
ACTGAGACACAGTCTTAGATATCACCATGGGATGGTCATG
TATCATCCTTTTTCTAGTAGCAACTGCAACCGGTGTACAT
TCTGACATCCAGATGACCCAGTCTCCATCCTCCCTGTCTG
CATCTGTAGGAGACAGAGTCACCATCACTTGCCAGGCGAG
TCAGGACATTAGCAACTATTTAAATTGGTATCAGCAGAAA
CCAGGGAAAGCCCCTAAGCTCCTGATCTACGATGCATCCA
ATTTGGAAACAGGGGTCCCATCAAGGTTCAGTGGAAGTGG
ATCTGGGACAGATTTTACTTTCACCATCAGCAGCCTGCAG
CCTGAAGATATTGCAACATATTACTGTCAACAGTATGAGT
TTATCGGCCCTGGGACCAAAGTGGATATCAAACGGGCTGA
TGCTGCACCAACTGTATCCATCTTCTCACCATCCAGTGAG
CAGTTAACATCTGGAGGTGCTTCAGTCGTGTGCTTCTTGA
ACAACTTCTACCCCAAAGACATCAATGTCAAGTGGAAGAT
TGATGGCAGTGAACGACAAAATGGCGTCCTGAACAGTTGG
ACTGATCAGGACAGCAAAGACAGCACCTACAGCATGAGCA
GCACCCTCACGTTGACCAAGGACGAGTATGAACGACATAA
CAGCTATACCTGTGAGGCCACTCACAAGACATCAACTTCA
CCCATTGTCAAGAGCTTCAACAGGAATGAGTGTAGGCGGA
AGCGGGGGTCAGGAGCAACCAACTTTTCTCTGCTGAAGCA
AGCCGGGGACGTAGAGGAAAACCCCGGACCCATGGGATGG
TCATGTATCATCCTTTTTCTAGTAGCAACTGCAACCGGTG
TACATTCTCAGGTCCATTTGTCACAGTCTGGGGCAGCGGT
GACGAAGCCCGGGGCCTCAGTGAGAGTCTCCTGCGAGGCT
TCCGGATACAAGATTAGTGACCACTTTATTCATTGGTGGC
GACAGGCCCCAGGACAGGGCCTTCAGTGGGTGGGGTGGAT
CAATCCTAAGACTGGTCAGCCAAACAATCCTCGTCAATTT
CAGGGTAGAGTCAGTCTGACTCGACAGGCGTCGTGGGACT
TTGACACATATTCCTTTTACATGGACCTCAAGGCAGTAAG
ATCGGACGACACGGCCATTTATTTCTGTGCGCGACAACGC
AGCGACTTTTGGGATTTCGACGTCTGGGGCAGCGGCACGC
AGGTCACTGTCTCGTCAGGTAAGCTGGCTTTTTTCTTTCT
GCACATTCCATTCTGAAACGGGATCGATTGGGAAATAAAC
TGTCTAGGGATCTCAGAGCCTTTAGGACAGATTATCTCCA
CATCTTTGAAAAACTAAGAATCTGTGTGATGGTGTTGGTG
GAGTCCCTGGATGATGGGATAGGGACTTTGGAGGCTCATT
TGAAGAAGATGCTAAAACAATCCTATGGCTGGAGGGATAG
TTGGGGCTGTAGTTGGAGATTTTCAGTTTTTAGAATAAAA
GTATTAGTTGTGGAATATACTTCAGGACCACCTCTGTGAC
AGCATTTATACAGTATCCGATGCATAGGGACAAAGAGTGG
AGTGGGGCACTTTCTTTAGATTTGTGAGGAATGTTCCGCA
CTAGATTGTTTAAAACTTCATTTGTTGGAAGGAGAGCTGT
CTTAGTGATTGAGTCAAGGGAGAAAGGCATCTAGCCTCGG
TCTCAAAAGGGTAGTTGCTGTCTAGAGAGGTCTGGTGGAG
CCTGCAAAAGTCCAGCTTTCAAAGGAACACAGAAGTATGT
GTATGGAATATTAGAAGATGTTGCTTTTACTCTTAAGTTG
GTTCCTAGGAAAAATAGTTAAATACTGTGACTTTAAAATG
TGAGAGGGTTTTCAAGTACTCATTTTTTTAAATGTCCAAA
ATTCTTGTCAATCAGTTTGAGGTCTTGTTTGTGTAGAACT
GATATTACTTAAAGTTTAACCGAGGAATGGGAGTGAGGCT
CTCTCATAACCTATTCAGAACTGACTTTTAACAATAATAA
ATTAAGTTTCAAATATTTTTAAATGAATTGAGCAATGTTG
AGTTGGAGTCAAGATGGCCTCGAGGAAT
(SEQ ID NO: 36)
Promoterless 3BNC60^{SI}, mouse
GCATAGCTAGCGCTCTTCAGTAAGAATGGCCTCTCCAGGT
CTTTATTTTTAACCTTTGTTATGGAGTTTTCTGAGCATTG
CAGACTAATCTTGGATATTTGTCCCTGAGGGAGCCGGCTG
AGAGAAGTTAAGAGTAGCAACAAGGAAATAGCAGGGTGTA
GAGGGATCTCCTGTCTGACAGGAGGCAAGAAGACAGATTC
TTACCCCTCCATTTCTCTTTTATCCCTCTCTGGTCCTCAG
AGGGAAGCGGAGAGGGCAGAGGAAGTCTGCTAACATGCGG
TGACGTCGAGGAGAATCCTGGACCTATGGGATGGTCATGT
ATCATCCTTTTTCTAGTAGCAACTGCAACCGGTGTACATT
CTGACATCCAGATGACCCAGTCTCCATCCTCCCTGTCTGC
ATCTGTAGGAGACAGAGTCACCATCACTTGCCAGGCGAGT
CAGGACATTAGCAACTATTTAAATTGGTATCAGCAGAAAC
CAGGGAAAGCCCCTAAGCTCCTGATCTACGATGCATCCAA
TTTGGAAACAGGGGTCCCATCAAGGTTCAGTGGAAGTGGA
TCTGGGACAGATTTTACTTTCACCATCAGCAGCCTGCAGC
CTGAAGATATTGCAACATATTACTGTCAACAGTATGAGTT
TATCGGCCCTGGGACCAAAGTGGATATCAAACGGGCTGAT
GCTGCACCAACTGTATCCATCTTCTCACCATCCAGTGAGC
AGTTAACATCTGGAGGTGCTTCAGTCGTGTGCTTCTTGAA
CAACTTCTACCCCAAAGACATCAATGTCAAGTGGAAGATT
GATGGCAGTGAACGACAAAATGGCGTCCTGAACAGTTGGA
CTGATCAGGACAGCAAAGACAGCACCTACAGCATGAGCAG
CACCCTCACGTTGACCAAGGACGAGTATGAACGACATAAC
AGCTATACCTGTGAGGCCACTCACAAGACATCAACTTCAC
CCATTGTCAAGAGCTTCAACAGGAATGAGTGTAGGCGGAA
GCGGGGGTCAGGAGCAACCAACTTTTCTCTGCTGAAGCAA
GCCGGGGACGTAGAGGAAAACCCCGGACCCATGGGATGGT
CATGTATCATCCTTTTTCTAGTAGCAACTGCAACCGGTGT
ACATTCTCAGGTCCATTTGTCACAGTCTGGGGCAGCGGTG
ACGAAGCCCGGGGCCTCAGTGAGAGTCTCCTGCGAGGCTT
CCGGATACAAGATTAGTGACCACTTTATTCATTGGTGGCG
ACAGGCCCCAGGACAGGGCCTTCAGTGGGTGGGGTGGATC
AATCCTAAGACTGGTCAGCCAAACAATCCTCGTCAATTTC
AGGGTAGAGTCAGTCTGACTCGACAGGCGTCGTGGGACTT
TGACACATATTCCTTTTACATGGACCTCAAGGCAGTAAGA
TCGGACGACACGGCCATTTATTTCTGTGCGCGACAACGCA
GCGACTTTTGGGATTTCGACGTCTGGGGCAGCGGCACGCA
GGTCACTGTCTCGTCAGGTAAGCTGGCTTTTTTCTTTCTG
CACATTCCATTCTGAAACGGGATCGATTGGGAAATAAACT
GTCTAGGGATCTCAGAGCCTTTAGGACAGATTATCTCCAC
ATCTTTGAAAAACTAAGAATCTGTGTGATGGTGTTGGTGG
AGTCCCTGGATGATGGGATAGGGACTTTGGAGGCTCATTT
GAAGAAGATGCTAAAACAATCCTATGGCTGGAGGGATAGT
TGGGGCTGTAGTTGGAGATTTTCAGTTTTTAGAATAAAAG
TATTAGTTGTGGAATATACTTCAGGACCACCTCTGTGACA
GCATTTATACAGTATCCGATGCATAGGGACAAAGAGTGGA
GTGGGGCACTTTCTTTAGATTTGTGAGGAATGTTCCGCAC
TAGATTGTTTAAAACTTCATTTGTTGGAAGGAGAGCTGTC
TTAGTGATTGAGTCAAGGGAGAAAGGCATCTAGCCTCGGT
CTCAAAAGGGTAGTTGCTGTCTAGAGAGGTCTGGTGGAGC
CTGCAAAAGTCCAGCTTTCAAAGGAACACAGAAGTATGTG
TATGGAATATTAGAAGATGTTGCTTTTACTCTTAAGTTGG
TTCCTAGGAAAAATAGTTAAATACTGTGACTTTAAAATGT
GAGAGGGTTTTCAAGTACTCATTTTTTTAAATGTCCAAAA
TTCTTGTCAATCAGTTTGAGGTCTTGTTTGTGTAGAACTG
ATATTACTTAAAGTTTAACCGAGGAATGGGAGTGAGGCTC
TCTCATAACCTATTCAGAACTGACTTTTAACAATAATAAA
TTAAGTTTCAAATATTTTTAAATGAATTGAGCAATGTTGA
GTTGGAGTCAAGATGGCCTCGAGATGA
(SEQ ID NO: 37)
10-1074, mouse
GCATAGCTAGCGCTCTTCAGTAAGAATGGCCTCTCCAGGT
CTTTATTTTTAACCTTTGTTATGGAGTTTTCTGAGCATTG
CAGACTAATCTTGGATATTTGTCCCTGAGGGAGCCGGCTG
AGAGAAGTTAAGAGTAGCAACAAGGAAATAGCAGGGTGTA
GAGGGATCTCCTGTCTGACAGGAGGCAAGAAGACAGATTC
TTACCCCTCCATTTCTCTTTTATCCCTCTCTGGTCCTCAG
AGAGTTAGAACTTGTTTATTGCAGCTTATAATGGTTACAA
ATAAAGCAATAGCATCACAAATTTCACAAATAAAGCATTT
TTTTCACTGCATTCTAGTTGTGGTTTGTCCAAACTCATCA
ATGTATCTTATCATGTCTGGTCGACAGTATGCAGAGGGCT
GTATCCACTGGAGAGGATGAAGTCACTGAGTTGGAAAACA
GAACAGGACAGGCACCTAACAAGTGGTTGCTATAGCCCAC
TGTTACCCTTTTACATGTATAGGCTCAGGATAAGCAGTGA
TACTGTGAGGTTTATGTGTGAGAACATCACAGTATAAACA
CATCTCAATAGAGGTCTTAGAGATCAGCACAATTAGTGAG
AAGTCATAAACAGTAGATACTATAAGGCATAGGCTCAGCT
ACCTAGGGTCAGGTATCTGTGTAAATCTGATTGTGTATCA
GGTTTAGATCAATATGACTTAGGGAGGCGAGTCATATGCA
AATCTAAGAAGACTTTAGAGAAGAAATCTGAGGCTCACCT
CACATAACAGCAAGAGAGTGTCCGGTTAGTCTCAAGGAAG
ACTGAGACACAGTCTTAGATATCACCATGGGATGGTCATG
TATCATCCTTTTTCTAGTAGCAACTGCAACCGGTGTACAT
TCTTCCTATGTGCGCCCGCTGTCAGTGGCCCTGGGGGAGA
CGGCCAGGATTTCCTGTGGACGACAGGCCCTTGGAAGTAG
AGCTGTTCAGTGGTATCAACATAGGCCAGGCCAGGCCCCT
ATATTGCTCATTTATAATAATCAAGACCGGCCCTCAGGGA
TCCCTGAGCGATTCTCTGGCACCCCTGATATTAATTTTGG
GACCAGGGCCACCCTGACCATCAGCGGGGTCGAAGCCGGG
GATGAAGCCGACTATTACTGTCACATGTGGGATAGTAGAA
GTGGCTTCAGTTGGTCTTTCGGCGGGGCGACCAGGCTGAC
CGTCCTACGGGCTGATGCTGCACCAACTGTATCCATCTTC
TCACCATCCAGTGAGCAGTTAACATCTGGAGGTGCTTCAG
TCGTGTGCTTCTTGAACAACTTCTACCCCAAAGACATCAA
TGTCAAGTGGAAGATTGATGGCAGTGAACGACAAAATGGC
GTCCTGAACAGTTGGACTGATCAGGACAGCAAAGACAGCA
CCTACAGCATGAGCAGCACCCTCACGTTGACCAAGGACGA
GTATGAACGACATAACAGCTATACCTGTGAGGCCACTCAC
AAGACATCAACTTCACCCATTGTCAAGAGCTTCAACAGGA
ATGAGTGTAGGCGGAAGCGGGGGTCAGGAGCAACCAACTT
TTCTCTGCTGAAGCAAGCCGGGGACGTAGAGGAAAACCCC
GGACCCATGGGATGGTCATGTATCATCCTTTTTCTAGTAG
CAACTGCAACCGGTGTACATTCTCAGGTGCAGCTGCAGGA
GTCGGGCCCAGGACTGGTGAAACCTTCGGAGACCCTGTCC
GTCACCTGCAGTGTCTCTGGAGATTCCATGAATAATTACT
ACTGGACTTGGATCCGGCAGTCCCCCGGAAAGGGACTGGA
GTGGATAGGCTATATCTCTGACAGAGAATCAGCGACTTAC
AACCCCTCCCTCAATAGTCGAGTCGTCATATCACGAGACA
CGTCGAAAAACCAATTGTCCCTAAAATTAAACTCCGTCAC
CCCTGCGGACACGGCCGTCTATTACTGTGCGACAGCGCGC
CGAGGACAGAGGATTTATGGAGTGGTTTCCTTTGGAGAGT
TCTTCTACTACTACTCCATGGACGTCTGGGGCAAGGGGAC
CACGGTCACCGTCTCCTCAGGTAAGCTGGCTTTTTTCTTT
CTGCACATTCCATTCTGAAACGGGATCGATTGGGAAATAA
ACTGTCTAGGGATCTCAGAGCCTTTAGGACAGATTATCTC
CACATCTTTGAAAAACTAAGAATCTGTGTGATGGTGTTGG
TGGAGTCCCTGGATGATGGGATAGGGACTTTGGAGGCTCA
TTTGAAGAAGATGCTAAAACAATCCTATGGCTGGAGGGAT
AGTTGGGGCTGTAGTTGGAGATTTTCAGTTTTTAGAATAA
AAGTATTAGTTGTGGAATATACTTCAGGACCACCTCTGTG
ACAGCATTTATACAGTATCCGATGCATAGGGACAAAGAGT
GGAGTGGGGCACTTTCTTTAGATTTGTGAGGAATGTTCCG
CACTAGATTGTTTAAAACTTCATTTGTTGGAAGGAGAGCT
GTCTTAGTGATTGAGTCAAGGGAGAAAGGCATCTAGCCTC
GGTCTCAAAAGGGTAGTTGCTGTCTAGAGAGGTCTGGTGG
AGCCTGCAAAAGTCCAGCTTTCAAAGGAACACAGAAGTAT
GTGTATGGAATATTAGAAGATGTTGCTTTTACTCTTAAGT
TGGTTCCTAGGAAAAATAGTTAAATACTGTGACTTTAAAA
TGTGAGAGGGTTTTCAAGTACTCATTTTTTTAAATGTCCA
AAATTCTTGTCAATCAGTTTGAGGTCTTGTTTGTGTAGAA
CTGATATTACTTAAAGTTTAACCGAGGAATGGGAGTGAGG
CTCTCTCATAACCTATTCAGAACTGACTTTTAACAATAAT
AAATTAAGTTTCAAATATTTTTAAATGAATTGAGCAATGT
TGAGTTGGAGTCAAGATGGCCTCGAGGAAT
(SEQ ID NO: 38)
3BNC117, mouse
GCATAGCTAGCGCTCTTCAGTAAGAATGGCCTCTCCAGGT
CTTTATTTTTAACCTTTGTTATGGAGTTTTCTGAGCATTG
CAGACTAATCTTGGATATTTGTCCCTGAGGGAGCCGGCTG
AGAGAAGTTAAGAGTAGCAACAAGGAAATAGCAGGGTGTA
GAGGGATCTCCTGTCTGACAGGAGGCAAGAAGACAGATTC
TTACCCCTCCATTTCTCTTTTATCCCTCTCTGGTCCTCAG
AGAGTTAGAACTTGTTTATTGCAGCTTATAATGGTTACAA
ATAAAGCAATAGCATCACAAATTTCACAAATAAAGCATTT
TTTTCACTGCATTCTAGTTGTGGTTTGTCCAAACTCATCA
ATGTATCTTATCATGTCTGGTCGACAGTATGCAGAGGGCT
GTATCCACTGGAGAGGATGAAGTCACTGAGTTGGAAAACA
GAACAGGACAGGCACCTAACAAGTGGTTGCTATAGCCCAC
TGTTACCCTTTTACATGTATAGGCTCAGGATAAGCAGTGA
TACTGTGAGGTTTATGTGTGAGAACATCACAGTATAAACA
CATCTCAATAGAGGTCTTAGAGATCAGCACAATTAGTGAG
AAGTCATAAACAGTAGATACTATAAGGCATAGGCTCAGCT
ACCTAGGGTCAGGTATCTGTGTAAATCTGATTGTGTATCA
GGTTTAGATCAATATGACTTAGGGAGGCGAGTCATATGCA
AATCTAAGAAGACTTTAGAGAAGAAATCTGAGGCTCACCT
CACATAACAGCAAGAGAGTGTCCGGTTAGTCTCAAGGAAG
ACTGAGACACAGTCTTAGATATCACCATGGGATGGTCATG
TATCATCCTTTTTCTAGTAGCAACTGCAACCGGTGTACAT
TCTGACATCCAGATGACCCAGTCTCCATCCTCCCTGTCTG
CCTCTGTGGGAGATACCGTCACTATCACTTGCCAGGCAAA
CGGCTACTTAAATTGGTATCAACAGAGGCGAGGGAAAGCC
CCAAAACTCCTGATCTACGATGGGTCCAAATTGGAAAGAG
GGGTCCCATCAAGGTTCAGTGGAAGAAGATGGGGGCAAGA
ATATAATCTGACCATCAACAATCTGCAGCCCGAAGACATT
GCAACATATTTTTGTCAAGTGTATGAGTTTGTCGTCCCTG
GGACCAGACTGGATTTGAAACGGGCTGATGCTGCACCAAC
TGTATCCATCTTCTCACCATCCAGTGAGCAGTTAACATCT
GGAGGTGCTTCAGTCGTGTGCTTCTTGAACAACTTCTACC
CCAAAGACATCAATGTCAAGTGGAAGATTGATGGCAGTGA
ACGACAAAATGGCGTCCTGAACAGTTGGACTGATCAGGAC
AGCAAAGACAGCACCTACAGCATGAGCAGCACCCTCACGT
TGACCAAGGACGAGTATGAACGACATAACAGCTATACCTG
TGAGGCCACTCACAAGACATCAACTTCACCCATTGTCAAG
AGCTTCAACAGGAATGAGTGTAGGCGGAAGCGGGGGTCAG
GAGCAACCAACTTTTCTCTGCTGAAGCAAGCCGGGGACGT
AGAGGAAAACCCCGGACCCATGGGATGGTCATGTATCATC
CTTTTTCTAGTAGCAACTGCAACCGGTGTACATTCTCAGG
TCCAATTGTTACAGTCTGGGGCAGCGGTGACGAAGCCCGG
GGCCTCAGTGAGAGTCTCCTGCGAGGCTTCTGGATACAAC
ATTCGTGACTACTTTATTCATTGGTGGCGACAGGCCCCAG
GACAGGGCCTTCAGTGGGTGGGATGGATCAATCCTAAGAC
AGGTCAGCCAAACAATCCTCGTCAATTTCAGGGTAGAGTC
AGTCTGACTCGACACGCGTCGTGGGACTTTGACACATTTT
CCTTTTACATGGACCTGAAGGCACTAAGATCGGACGACAC
GGCCGTTTATTTCTGTGCGCGACAGCGCAGCGACTATTGG
GATTTCGACGTCTGGGGCAGTGGAACCCAGGTCACTGTCT
CGTCAGGTAAGCTGGCTTTTTTCTTTCTGCACATTCCATT
CTGAAACGGGATCGATTGGGAAATAAACTGTCTAGGGATC
TCAGAGCCTTTAGGACAGATTATCTCCACATCTTTGAAAA
ACTAAGAATCTGTGTGATGGTGTTGGTGGAGTCCCTGGAT
GATGGGATAGGGACTTTGGAGGCTCATTTGAAGAAGATGC
TAAAACAATCCTATGGCTGGAGGGATAGTTGGGGCTGTAG
TTGGAGATTTTCAGTTTTTAGAATAAAAGTATTAGTTGTG
GAATATACTTCAGGACCACCTCTGTGACAGCATTTATACA
GTATCCGATGCATAGGGACAAAGAGTGGAGTGGGGCACTT
TCTTTAGATTTGTGAGGAATGTTCCGCACTAGATTGTTTA
AAACTTCATTTGTTGGAAGGAGAGCTGTCTTAGTGATTGA
GTCAAGGGAGAAAGGCATCTAGCCTCGGTCTCAAAAGGGT
AGTTGCTGTCTAGAGAGGTCTGGTGGAGCCTGCAAAAGTC
CAGCTTTCAAAGGAACACAGAAGTATGTGTATGGAATATT
AGAAGATGTTGCTTTTACTCTTAAGTTGGTTCCTAGGAAA
AATAGTTAAATACTGTGACTTTAAAATGTGAGAGGGTTTT
CAAGTACTCATTTTTTTAAATGTCCAAAATTCTTGTCAAT
CAGTTTGAGGTCTTGTTTGTGTAGAACTGATATTACTTAA
AGTTTAACCGAGGAATGGGAGTGAGGCTCTCTCATAACCT
ATTCAGAACTGACTTTTAACAATAATAAATTAAGTTTCAA
ATATTTTTAAATGAATTGAGCAATGTTGAGTTGGAGTCAA
GATGGCCTCGAGGAAT
(SEQ ID NO: 39)
PGT121, mouse
GCATAGCTAGCGCTCTTCAGTAAGAATGGCCTCTCCAGGT
CTTTATTTTTAACCTTTGTTATGGAGTTTTCTGAGCATTG
CAGACTAATCTTGGATATTTGTCCCTGAGGGAGCCGGCTG
AGAGAAGTTAAGAGTAGCAACAAGGAAATAGCAGGGTGTA
GAGGGATCTCCTGTCTGACAGGAGGCAAGAAGACAGATTC
TTACCCCTCCATTTCTCTTTTATCCCTCTCTGGTCCTCAG
AGAGTTAGAACTTGTTTATTGCAGCTTATAATGGTTACAA
ATAAAGCAATAGCATCACAAATTTCACAAATAAAGCATTT
TTTTCACTGCATTCTAGTTGTGGTTTGTCCAAACTCATCA
ATGTATCTTATCATGTCTGGTCGACAGTATGCAGAGGGCT
GTATCCACTGGAGAGGATGAAGTCACTGAGTTGGAAAACA
GAACAGGACAGGCACCTAACAAGTGGTTGCTATAGCCCAC
TGTTACCCTTTTACATGTATAGGCTCAGGATAAGCAGTGA
TACTGTGAGGTTTATGTGTGAGAACATCACAGTATAAACA
CATCTCAATAGAGGTCTTAGAGATCAGCACAATTAGTGAG
AAGTCATAAACAGTAGATACTATAAGGCATAGGCTCAGCT
ACCTAGGGTCAGGTATCTGTGTAAATCTGATTGTGTATCA
GGTTTAGATCAATATGACTTAGGGAGGCGAGTCATATGCA
AATCTAAGAAGACTTTAGAGAAGAAATCTGAGGCTCACCT
CACATAACAGCAAGAGAGTGTCCGGTTAGTCTCAAGGAAG
ACTGAGACACAGTCTTAGATATCACCATGGGATGGTCATG
TATCATCCTTTTTCTAGTAGCAACTGCAACCGGTGTACAT
TCTTCCGATATATCTGTGGCCCCAGGAGAGACGGCCAGGA
TTTCCTGTGGGGAAAAGAGCCTTGGAAGTAGAGCTGTACA
ATGGTATCAACACAGGGCCGGCCAGGCCCCCTCTTTAATC
ATATATAATAATCAGGACCGGCCCTCAGGGATCCCTGAGC
GATTCTCTGGCTCCCCTGACTCCCCTTTTGGGACCACGGC
CACCCTGACCATCACCAGTGTCGAAGCCGGGGATGAGGCC
GACTATTACTGTCATATATGGGATAGTAGAGTTCCCACCA
AATGGGTCTTCGGCGGAGGGACCACGCTGACCGTGTTACG
GGCTGATGCTGCACCAACTGTATCCATCTTCTCACCATCC
AGTGAGCAGTTAACATCTGGAGGTGCTTCAGTCGTGTGCT
TCTTGAACAACTTCTACCCCAAAGACATCAATGTCAAGTG
GAAGATTGATGGCAGTGAACGACAAAATGGCGTCCTGAAC
AGTTGGACTGATCAGGACAGCAAAGACAGCACCTACAGCA
TGAGCAGCACCCTCACGTTGACCAAGGACGAGTATGAACG
ACATAACAGCTATACCTGTGAGGCCACTCACAAGACATCA
ACTTCACCCATTGTCAAGAGCTTCAACAGGAATGAGTGTA
GGCGGAAGCGGGGGTCAGGAGCAACCAACTTTTCTCTGCT
GAAGCAAGCCGGGGACGTAGAGGAAAACCCCGGACCCATG
GGATGGTCATGTATCATCCTTTTTCTAGTAGCAACTGCAA
CCGGTGTACATTCTCAGATGCAGTTACAGGAGTCGGGCCC
CGGACTGGTGAAGCCTTCGGAAACCCTGTCCCTCACGTGC
AGTGTGTCTGGTGCCTCCATAAGTGACAGTTACTGGAGCT
GGATCCGGCGGTCCCCAGGGAAGGGACTTGAGTGGATTGG
GTATGTCCACAAAAGCGGCGACACAAATTACAGCCCCTCC
CTCAAGAGTCGAGTCAACTTGTCGTTAGACACGTCCAAAA
ATCAGGTGTCCCTGAGCCTTGTGGCCGCGACCGCTGCGGA
CTCGGGCAAATATTATTGCGCGAGAACACTGCACGGGAGG
AGAATTTATGGAATCGTTGCCTTCAATGAGTGGTTCACCT
ACTTCTACATGGACGTCTGGGGCAATGGGACTCAGGTCAC
CGTCTCCTCAGGTAAGCTGGCTTTTTTCTTTCTGCACATT
CCATTCTGAAACGGGATCGATTGGGAAATAAACTGTCTAG
GGATCTCAGAGCCTTTAGGACAGATTATCTCCACATCTTT
GAAAAACTAAGAATCTGTGTGATGGTGTTGGTGGAGTCCC
TGGATGATGGGATAGGGACTTTGGAGGCTCATTTGAAGAA
GATGCTAAAACAATCCTATGGCTGGAGGGATAGTTGGGGC
TGTAGTTGGAGATTTTCAGTTTTTAGAATAAAAGTATTAG
TTGTGGAATATACTTCAGGACCACCTCTGTGACAGCATTT
ATACAGTATCCGATGCATAGGGACAAAGAGTGGAGTGGGG
CACTTTCTTTAGATTTGTGAGGAATGTTCCGCACTAGATT
GTTTAAAACTTCATTTGTTGGAAGGAGAGCTGTCTTAGTG
ATTGAGTCAAGGGAGAAAGGCATCTAGCCTCGGTCTCAAA
AGGGTAGTTGCTGTCTAGAGAGGTCTGGTGGAGCCTGCAA
AAGTCCAGCTTTCAAAGGAACACAGAAGTATGTGTATGGA
ATATTAGAAGATGTTGCTTTTACTCTTAAGTTGGTTCCTA
GGAAAAATAGTTAAATACTGTGACTTTAAAATGTGAGAGG
GTTTTCAAGTACTCATTTTTTTAAATGTCCAAAATTCTTG
TCAATCAGTTTGAGGTCTTGTTTGTGTAGAACTGATATTA
CTTAAAGTTTAACCGAGGAATGGGAGTGAGGCTCTCTCAT
AACCTATTCAGAACTGACTTTTAACAATAATAAATTAAGT
TTCAAATATTTTTAAATGAATTGAGCAATGTTGAGTTGGA
GTCAAGATGGCCTCGAGGAAT
(SEQ ID NO: 40)
3BNC60^{SI}, human
GCATAGCTAGCGCTCTTCAACCACGGTCACCGTCTCCTCA
GGTAAGAATGGCCACTCTAGGGCCTTTGTTTTCTGCTACT
GCCTGTGGGGTTTCCTGAGCATTGCAGGTTGGTCCTCGGG
GCATGTTCCGAGGGGACCTGGGCGGACTGGCCAGGAGGGG
ATGGGCACTGGGGTGCCTTGAGGATCTGGGAGCCTCTGAC
AGCGGGACGCAAGTAGTGAGGGCACTCAGAACGCCACTCA
GCCCCGACAGGCAGGGCACGAGGAGGCAGCTCCTCACCCT
CCCTTTCTCTTTTGTCCTGCGGGTCCTCAGGGAGTTAGAA
CTTGTTTATTGCAGCTTATAATGGTTACAAATAAAGCAAT
AGCATCACAAATTTCACAAATAAAGCATTTTTTTCACTGC
ATTCTAGTTGTGGTTTGTCCAAACTCATCAATGTATCTTA
TCATGTCTGGTAACGAGTGGCCACCTTTTCAGTGTTACCA
GTGAGCTCTGAGTGTTCCTAATGGGACCAGGATGGGTCTA
GGTGCCTGCTCAATGTCAGAGACAGCAATGGTCCCACAAA
AAACCCAGGTAATCTTTAGGCCAATAAAATGTGGGTTCAC
AGTGAGGAGTGCATCCTGGGGTTGGGGTTTGTTCTGCAGC
GGGAAGAGTGCTGTGCACAGAAAGCTTAGAAATGGGGCAA
GAGATGCTTTTCCTCAGGCAGGATTTAGGGCTTGGTCTCT
CAGCATCCCACACTTGTACAGCTGATGTGGCATCTGTGTT
TTCTTTCTCATCCTAGATCAGGCTTTGAGCTGTGAAATAC
CCTGCCTCATGCATATGCAAATAACCTGAGGTCTTCTGAG
ATAAATATAGATATATTGGTGCCCTGAGAGCATCACATAA
CAACCACATTCCTCCTCTGAAGAAGCCCCTGGGAGCACAG
CTCATCACCATGGGATGGTCATGTATCATCCTTTTTCTAG
TAGCAACTGCAACCGGTGTACATTCTGACATCCAGATGAC
CCAGTCTCCATCCTCCCTGTCTGCATCTGTAGGAGACAGA
GTCACCATCACTTGCCAGGCGAGTCAGGACATTAGCAACT
ATTTAAATTGGTATCAGCAGAAACCAGGGAAAGCCCCTAA
GCTCCTGATCTACGATGCATCCAATTTGGAAACAGGGGTC
CCATCAAGGTTCAGTGGAAGTGGATCTGGGACAGATTTTA
CTTTCACCATCAGCAGCCTGCAGCCTGAAGATATTGCAAC
ATATTACTGTCAACAGTATGAGTTTATCGGCCCTGGGACC
AAAGTGGATATCAAACGAACTGTGGCTGCACCATCTGTCT
TCATCTTCCCGCCATCTGATGAGCAGTTGAAATCTGGAAC
TGCCTCTGTTGTGTGCCTGCTGAATAACTTCTATCCCAGA
GAGGCCAAAGTACAGTGGAAGGTGGATAACGCTCTCCAAA
GCGGTAACTCCCAGGAGAGTGTCACAGAGCAGGACAGCAA
GGACAGCACCTACAGCCTCAGCAGCACCCTGACGCTGAGC
AAAGCAGACTACGAGAAACACAAAGTCTACGCCTGCGAAG
TCACCCATCAGGGCCTGAGCTCGCCCGTCACAAAGAGCTT
CAACAGGGGAGAGTGTAGGCGGAAGCGGGGGTCAGGAGCA
ACCAACTTTTCTCTGCTGAAGCAAGCCGGGGACGTAGAGG
AAAACCCCGGACCCATGGGATGGTCATGTATCATCCTTTT
TCTAGTAGCAACTGCAACCGGTGTACATTCTCAGGTCCAT
TTGTCACAGTCTGGGGCAGCGGTGACGAAGCCCGGGGCCT
CAGTGAGAGTCTCCTGCGAGGCTTCCGGATACAAGATTAG
TGACCACTTTATTCATTGGTGGCGACAGGCCCCAGGACAG
GGCCTTCAGTGGGTGGGGTGGATCAATCCTAAGACTGGTC
AGCCAAACAATCCTCGTCAATTTCAGGGTAGAGTCAGTCT
GACTCGACAGGCGTCGTGGGACTTTGACACATATTCCTTT
TACATGGACCTCAAGGCAGTAAGATCGGACGACACGGCCA
TTTATTTCTGTGCGCGACAACGCAGCGACTTTTGGGATTT
CGACGTCTGGGGCAGCGGCACGCAGGTCACTGTCTCGTCA
GGTGAGTCCTCACAACCTCTCTCCTGCTTTAACTCTGAAG
GGTTTTGCTGCTGGATTTTCCGATGCCTTTGGAAAATGGG
ACTCAGGTTGGGTGCGTCTGATGGAGTAACTGAGCCTGGG
GGCTTGGGGAGCCACATTTGGACGAGATGCCTGAACAAAC
CAGGGGTCTTAGTGATGGCTGAGGAATGTGTCTCAGGAGC
GGTGTCTGTAGGACTGCAAGATCGCTGCACAGCAGCGAAT
CGTGAAATATTTTCTTTAGAATTATGAGGTGCGCTGTGTG
TCAACCTGCATCTTAAATTCTTTATTGGCTGGAAAGAGAA
CTGTCGGAGTGGGTGAATCCAGCCAGGAGGGACGCGTAGC
CCCGGTCTTGATGAGAGCAGGGTTGGGGGCAGGGGTAGCC
CAGAAACGGTGGCTGCCGTCCTGACAGGGGCTTAGGGAGG
CTCCAGGACCTCAGTGCCTTGAAGCTGGTTTCCATGAGAA
AAGGATTGTTTATCTTAGGAGGCATGCTTACTGTTAAAAG
ACAGGATATGTTTGAAGTGGCTTCTGAGAAAAATGGTTAA
GAAAATTATGACTCGAGGAATT
  (SEQ ID NO: 41)
10-1074, human
GCATAGCTAGCGCTCTTCAACCACGGTCACCGTCTCCTCA
GGTAAGAATGGCCACTCTAGGGCCTTTGTTTTCTGCTACT
GCCTGTGGGGTTTCCTGAGCATTGCAGGTTGGTCCTCGGG
GCATGTTCCGAGGGGACCTGGGCGGACTGGCCAGGAGGGG
ATGGGCACTGGGGTGCCTTGAGGATCTGGGAGCCTCTGAC
AGCGGGACGCAAGTAGTGAGGGCACTCAGAACGCCACTCA
GCCCCGACAGGCAGGGCACGAGGAGGCAGCTCCTCACCCT
CCCTTTCTCTTTTGTCCTGCGGGTCCTCAGGGAGTTAGAA
CTTGTTTATTGCAGCTTATAATGGTTACAAATAAAGCAAT
AGCATCACAAATTTCACAAATAAAGCATTTTTTTCACTGC
ATTCTAGTTGTGGTTTGTCCAAACTCATCAATGTATCTTA
TCATGTCTGGTAACGAGTGGCCACCTTTTCAGTGTTACCA
GTGAGCTCTGAGTGTTCCTAATGGGACCAGGATGGGTCTA
GGTGCCTGCTCAATGTCAGAGACAGCAATGGTCCCACAAA
AAACCCAGGTAATCTTTAGGCCAATAAAATGTGGGTTCAC
AGTGAGGAGTGCATCCTGGGGTTGGGGTTTGTTCTGCAGC
GGGAAGAGTGCTGTGCACAGAAAGCTTAGAAATGGGGCAA
GAGATGCTTTTCCTCAGGCAGGATTTAGGGCTTGGTCTCT
CAGCATCCCACACTTGTACAGCTGATGTGGCATCTGTGTT
TTCTTTCTCATCCTAGATCAGGCTTTGAGCTGTGAAATAC
CCTGCCTCATGCATATGCAAATAACCTGAGGTCTTCTGAG
ATAAATATAGATATATTGGTGCCCTGAGAGCATCACATAA
CAACCACATTCCTCCTCTGAAGAAGCCCCTGGGAGCACAG
CTCATCACCATGGGATGGTCATGTATCATCCTTTTTCTAG
TAGCAACTGCAACCGGTGTACATTCTTCCTATGTGCGCCC
GCTGTCAGTGGCCCTGGGGGAGACGGCCAGGATTTCCTGT
GGACGACAGGCCCTTGGAAGTAGAGCTGTTCAGTGGTATC
AACATAGGCCAGGCCAGGCCCCTATATTGCTCATTTATAA
TAATCAAGACCGGCCCTCAGGGATCCCTGAGCGATTCTCT
GGCACCCCTGATATTAATTTTGGGACCAGGGCCACCCTGA
CCATCAGCGGGGTCGAAGCCGGGGATGAAGCCGACTATTA
CTGTCACATGTGGGATAGTAGAAGTGGCTTCAGTTGGTCT
TTCGGCGGGGCGACCAGGCTGACCGTCCTACGAACTGTGG
CTGCACCATCTGTCTTCATCTTCCCGCCATCTGATGAGCA
GTTGAAATCTGGAACTGCCTCTGTTGTGTGCCTGCTGAAT
AACTTCTATCCCAGAGAGGCCAAAGTACAGTGGAAGGTGG
ATAACGCTCTCCAAAGCGGTAACTCCCAGGAGAGTGTCAC
AGAGCAGGACAGCAAGGACAGCACCTACAGCCTCAGCAGC
ACCCTGACGCTGAGCAAAGCAGACTACGAGAAACACAAAG
TCTACGCCTGCGAAGTCACCCATCAGGGCCTGAGCTCGCC
CGTCACAAAGAGCTTCAACAGGGGAGAGTGTAGGCGGAAG
CGGGGGTCAGGAGCAACCAACTTTTCTCTGCTGAAGCAAG
CCGGGGACGTAGAGGAAAACCCCGGACCCATGGGATGGTC
ATGTATCATCCTTTTTCTAGTAGCAACTGCAACCGGTGTA
CATTCTCAGGTGCAGCTGCAGGAGTCGGGCCCAGGACTGG
TGAAACCTTCGGAGACCCTGTCCGTCACCTGCAGTGTCTC
TGGAGATTCCATGAATAATTACTACTGGACTTGGATCCGG
CAGTCCCCCGGAAAGGGACTGGAGTGGATAGGCTATATCT
CTGACAGAGAATCAGCGACTTACAACCCCTCCCTCAATAG
TCGAGTCGTCATATCACGAGACACGTCGAAAAACCAATTG
TCCCTAAAATTAAACTCCGTCACCCCTGCGGACACGGCCG
TCTATTACTGTGCGACAGCGCGCCGAGGACAGAGGATTTA
TGGAGTGGTTTCCTTTGGAGAGTTCTTCTACTACTACTCC
ATGGACGTCTGGGGCAAGGGGACCACGGTCACCGTCTCCT
CAGGTGAGTCCTCACAACCTCTCTCCTGCTTTAACTCTGA
AGGGTTTTGCTGCTGGATTTTCCGATGCCTTTGGAAAATG
GGACTCAGGTTGGGTGCGTCTGATGGAGTAACTGAGCCTG
GGGGCTTGGGGAGCCACATTTGGACGAGATGCCTGAACAA
ACCAGGGGTCTTAGTGATGGCTGAGGAATGTGTCTCAGGA
GCGGTGTCTGTAGGACTGCAAGATCGCTGCACAGCAGCGA
ATCGTGAAATATTTTCTTTAGAATTATGAGGTGCGCTGTG
TGTCAACCTGCATCTTAAATTCTTTATTGGCTGGAAAGAG
AACTGTCGGAGTGGGTGAATCCAGCCAGGAGGGACGCGTA
GCCCCGGTCTTGATGAGAGCAGGGTTGGGGGCAGGGGTAG
CCCAGAAACGGTGGCTGCCGTCCTGACAGGGGCTTAGGGA
GGCTCCAGGACCTCAGTGCCTTGAAGCTGGTTTCCATGAG
AAAAGGATTGTTTATCTTAGGAGGCATGCTTACTGTTAAA
AGACAGGATATGTTTGAAGTGGCTTCTGAGAAAAATGGTT
AAGAAAATTATGACTCGAGGAATT
(SEQ ID NO: 42)
```

Murine Cell Culture

Mature, resting B cells were obtained from mouse spleens by forcing tissue through a 70 μm mesh into PBS containing 2% heat-inactivated fetal bovine serum (FBS). After ACK lysis for 3 min, untouched B cells were enriched using anti-CD43 magnetic beads (MACS) according to manufacturer's protocol (Miltenyi Biotec) obtaining >95% purity. 3.2×10⁷ cells/10 cm dish (Gibco) were cultured at 37° C. 5% CO2 in 10 mL mouse B cell medium consisting of RPMI-1640, supplemented with 10% heat-inactivated FBS, 10 mM HEPES, antibiotic-antimycotic (1×), 1 mM sodium pyruvate, 2 mM L-glutamine and 53 μM 2-mercaptoethanol (all from Gibco) and activated with 2 μg/mL anti-mouse RP105 clone RP/14 (produced in house or BD Pharmingen Cat. #562191).

NB-21 feeder cells (Kuraoka et al., 2016) were maintained in DMEM supplemented with 10% heat-inactivated FBS and antibiotic-antimycotic (1×). For co-culture, feeder cells were irradiated with 80 Gy and seeded simultaneously with B cells, 24 h after transfection, into B cell culture medium supplemented with 1 ng/mL recombinant mouse IL-4 (PeproTech Ca. #214-14) and 2 μg/mL anti-mouse RP105 clone RP/14.

Human Cell Culture

Leukapheresis samples of healthy human individuals were collected after signed informed consent in accordance with protocol TSC-0910 approved by the Rockefeller University Institutional Review Board (IRB). PBMCs were prepared, stored in liquid nitrogen, then thawed in a 37° C. water bath and resuspended in human B cell medium composed of RPMI-1640, supplemented with 10% heat-inactivated FBS or human serum, 10 mM HEPES, antibiotic-antimycotic (1×), 1 mM sodium pyruvate, 2 mM L-glutamine and 53 2-mercaptoethanol (all from Gibco). B cells were isolated using EasySep human naïve B cell Enrichment Kit (Stemcell Cat. #19254) according to the manufacturer's instructions and cultured in the above medium supplemented with 2 μg/mL anti-human RP105 antibody clone MHR73-11 (BioLegend Cat. #312907).

RNP Preparation and Transfection

Per 100 μL transfection, 1 μL of 200 μM crRNA and 1 μL 200 μM tracrRNA in duplex buffer (all IDT) were mixed, denatured at 95° C. for 5 min, re-natured for 5 min at room temperature. 5.6 μL PBS and 2.4 μL 61 μM Cas9 V3 (IDT, Cat. #1081059) were added and incubated for 15-30 min. If required RNPs were mixed at the following ratios: 50% crIgH, 25% crIgK₁ and 2 5% crIgK₂ (mouse) or 50% crhIgH₃ and 50% crhIgK₃ (human). 4 μL 100 μM electroporation enhancer in duplex buffer or 4 μL HDRT at 2.5 μg/μL were added to 10 μL mixed RNP and incubated for a further 1-2 min.

Alternatively, per 10 μL transfection, 1.875 μL of 100 μM sgRNA in duplex buffer (all IDT) were mixed with 1 μL 61 μM Cas9 V3 (IDT, Cat. #1081059) and incubated for 15-30 min at room temperature. If required RNPs were mixed at the following ratios: 50% sgIgH, 50% sgIgK₁ (mouse) or sgIgKJ₁

24 h after stimulation, activated mouse or human B cells were harvested, washed once in PBS and resuspended in Mouse B cell Nucleofector Solution with Supplement (murine B cells) or Primary Cell Nucleofector Solution 3 with Supplement (human B cells) prepared to the manufacturer's instructions (Lonza) at a concentration of 4-5×10⁶ cells/86 μL. 86 μL cells were added to the RNP/HPRT mix, gently mixed by pipetting and transferred into nucleofection cuvettes and electroporated using an Amaxa IIb machine setting Z-001 (murine B cells) or Amaxa 4D machine setting EH-140 (human B cells). Cells were immediately transferred into 6-well dishes containing 5 mL prewarmed mouse or human B cell medium supplemented with the relevant anti-RP105 antibody at 2 μg/mL and incubated at 37° C. 5% CO2 for 24 h before further processing.

Alternatively, 24-48 h after stimulation, activated mouse or human B cells were harvested, washed once in PBS and resuspended buffer R (mouse) or buffer T (human) from the Neon transfection kit at 55.55×10⁶ cells/mL. 9 uL cells were mixed with 1.15 uL RNP and transfected in 10 uL Neon tips at 1350V or 1650V, 15 or 20 ms, 1p (mouse) or 1750V 20 ms 1p (human) using the Neon Transfection system. Cells were ejected into 50 uL prewarmed medium without antibiotics with or without AAV HDR donor template in a 48-well plate. 5 h later an additional 450 uL medium with antibiotics and stimulants was added. For 100 uL Neon tips all volumes were scaled by a factor of 10 and cells were ejected into 6-well plates.

HDR by AAV Transduction

Immediately after RNP transfections, cells were transferred into B cell medium to a concentration of 8.33×10\hat{ }6 cells/mL. The B cell medium without antibiotics contained stimulants as for activation and AAV6 HDRT at a ratio of 125,000-1,000,000 AAV genome copies/cell. Cells were incubated with the concentrated AAV at 37° C., 5% CO2 for 4-10 h then 9-fold volume of full B medium with antibiotics and stimulants was added and cells were further incubated at 37° C., 5% CO2 until further processing

TIDE Assay

Genomic DNA was extracted from 0.5-5×10⁵ cells by standard phenol/chloroform extraction 24-42 h after transfection. PCRs to amplify human or mouse Ig loci targeted by CRISPR-Cas9 were performed using Phusion Green Hot Start II High-Fidelity polymerase (Thermo Fisher Cat. #F-537L) and primers listed in Table 3. Thermocycler was set to 40 cycles, annealing at 65° C. for 30 s and extending at 72° C. for 30 s. PCR product size was verified by gel electrophoresis, bands gel-extracted and sent for Sanger sequencing (Genewiz) using the relevant PCR primers. ab 1 files were analyzed using the TIDE web tool (tide.nki.nl) using samples receiving scramble or irrelevant HPRT-targeting crRNA as reference (Brinkman et al., 2014).

TABLE 3
Primers for TIDE analysis

| Forward | SEQ | Reverse | SEQ | |
|---|---|---|---|---|
| primer | ID | primer | ID | |
| 5′ to 3′ | NO | 5′ to 3′ | NO | |
| sequence | | sequence | | Comments |
| CCTGGCCCCA | 43 | GCGTCTCAGG | 44 | for TIDE analysis |
| TTGTTCCTTA | | ACCTTTGTCT | | mouse Igkc, product |
| | | | | 483 bp |
| | | | | for TIDE analysis |
| | | | | mouse J_{H}4 intron, |
| | | | | product 495 bp |
| | | | | for TIDE analysis |
| | | | | human IGKC, |
| | | | | product 638 bp |
| | | | | for TIDE analysis |
| | | | | human IGKC, |
| | | | | product 515 bp |
| | | | | for TIDE analysis |
| | | | | human J_{H}6 intron, |
| | | | | product 563 bp |
| | | | | for TIDE analysis |
| | | | | human J_{H}6 intron, |
| | | | | product 533 bp |
| AATGTCTGAG | 45 | TGTCACAGAG | 46 | |
| TTGCCCAGGG | | GTGGTCCTGA | | |
| ATGGCTGCAA | 47 | GGAAAAAGGG | 48 | |
| AGAGCTCCAA | | TCAGAGGCCA | | |
| TGCCCTGTGA | 49 | GAGCTGGAGG | 50 | |
| TTATCCGCAA | | ACCGCAATAG | | |
| GCCACTCTAG | 51 | AGCTTCAAGG | 52 | |
| GGCCTTTGTT | | CACTGAGGTC | | |
| CTACATGGAC | 53 | CTGCTCTCAT | 54 | |
| GTCTGGGGC | | CAAGACCGGG | | |

Flow Cytometry

Mouse spleens were forced through a 70 μm mesh into FACS buffer (PBS containing 2% heat-inactivated FBS and 2 mM EDTA) and red blood cells were lysed in ACK lysing buffer (Gibco) for 3 min. Cultured cells were harvested by centrifugation. Then cells were washed and Fc-receptors blocked for 15 min on ice. Cells were stained for 20 min on ice with antibodies or reagents listed in Table 4 and depending on the stain, washed again and secondary stained for another 20 min on ice before acquisition on a BD LSRFortessa. For intracellular FIX stain, cells were fixed after surface staining and washing with PBS using Reagent A (Nordic MUbio, Cat #GAS-002-1) for 15 min at room temperature. Then cells were washed with PBS followed by permeabilization with Reagent B containing 1 ug/ml FITC-conjugated Factor 9 polyclonal antibody (Affinity Biologicals, Cat #GAFIX-APFTC). After 15 min at room temperature, cells were washed with PBS, resuspended in FACS buffer and acquired on a BD LSRFortessa. Anti-idiotype 3BNC60^{SI} (iv8) produced as human IgG1/κ was detected with anti-human Igκ-BV421 on edited mouse B cells. B1-8hi BCR expression was detected using NP-conjugated streptavidin-AlexaFluor647. GC B cells were gated as single/live, B220⁺, CD38⁻ FAS⁺, GL7⁺, IgD⁻. Allotypic markers CD45.1 and CD45.2 were used to track adoptively transferred B cells.

TABLE 4
Flow cytometric reagents

| | Target | Antibody | Company/ | |
|---|---|---|---|---|
| Reagent | species | clone | Source | Cat. # |
| CD16/32 | mouse | 2.4G2 | BD Biosciences | 7248907 |
| CD4-eF780 | mouse | RM4-5 | Thermo Fisher | 47-0042-82 |
| CD8a-eF780 | mouse | 53-6.7 | Thermo Fisher | 47-0081-82 |
| NK1.1-eF780 | mouse | PK136 | Thermo Fisher | 47-5941-82 |
| F4/80-eF780 | mouse | BM8 | Thermo Fisher | 47-4801-82 |
| LY6G (Gr1)-eF780 | mouse | RB6-8C5 | Thermo Fisher | 47-5931-82 |
| IgG1-APC | mouse | A85-1 | BD Pharmingen | 560089 |
| CD95 (FAS)-PE-Cy7 | mouse | Jo2 | BD Biosciences | 557653 |
| CD45.2-PE | mouse | 104 | BioLegend | 109808 |
| CD45.1-BV421 | mouse | A20 | BioLegend | 110732 |
| GL7-FITC | mouse | GL7 | BD Pharmingen | 553666 |
| IgD-BV786 | mouse | 11-26c.2a | BD Horizon | 563618 |
| CD45R/B220-BV605 | mouse/human | RA3-6B2 | BioLegend | 103244 |
| CD19-PECy7 | mouse | 6D5 | BioLegend | 115520 |
| IgM^{a}-FITC | mouse | DS-1 | BD Pharmingen | 553516 |
| IgM^{b}-PE | mouse | AF6-78 | BioLegend | 406208 |
| Ig light chain λ-APC | mouse | RML-42 | BioLegend | 407306 |
| Ig light chain κ-BV421 | mouse | 187.1 | BD Horizon | 562888 |
| IgM Fab-FITC | mouse | polyclonal | Jackson Immunoresearch | 115-097-020 |
| Zombie NIR | N/A* | N/A | BioLegend | 423105 |
| Streptavidin-PE | N/A | N/A | BD Pharmingen | 554061 |
| Streptavidin-BV421 | N/A | N/A | BD Horizon | 563259 |
| NP-streptavidin-AF647 | N/A | N/A | in house | N/A |
| TM4 core-biotin | N/A | N/A | in house (McGuire 2014) | N/A |
| 10mut-biotin | N/A | N/A | in house (Steichen 2016) | N/A |
| anti-3BNC60^{SI} idiotype | N/A | Iv8 | in house, this publication | N/A |
| Human Fc Block | human | N/A | BD Horizon | 564220 |
| Ig light chain λ-APC | human | MHL38 | BioLegend | 316609 |
| CD19-PECy7 | human | SJ25C1 | BioLegend | 363011 |
| IgM-FITC | human | MHM88 | BioLegend | 314506 |
| IgD-BV785 | human | IA6-2 | BioLegend | 348241 |
| Ig light chain κ-BV421 | human | MHK-49 | BioLegend | 316518 |

Mice

C57BL/6J and B6.Igha (B6.Cg-Gpila Thy1a Igha/J) and B6.SJL were obtained from the Jackson Laboratory. Igha/b mice were obtained by intercrossing B6.Igha and B6.SJL mice. B1-8hi (Shih et al., 2002), 3BNC60SI (Dosenovic et al., 2018) and PGT121 (Escolano et al., 2016; Steichen et al., 2016) strains were generated and maintained in our laboratory on a C57BL/6J background. All experiments used age and sex-matched animals, littermates when possible. All experiments were performed with authorization from the Institutional Review Board and the Rockefeller University IACUC.

Cell Transfers and Immunizations

After culture, mouse B cells were harvested at the indicated time points and resuspended in mouse B cell medium without anti-RP105 antibody and rested for 2-3 h at 37° C., 5% CO2. Then cells were washed once in PBS and resuspended in 200 μL PBS/mouse containing the indicated number of initially transfected cells. 200 μL cell suspension/mouse were injected intravenously via the retroorbital sinus. Number of transferred, edited B cells was estimated as follows: Number of cells transfected×20% survival×0.15-0.4% transfection efficiency×50% handling/proliferation×5% transfer efficiency (Dosenovic et al., 2018). Mice were immunized intraperitoneally within 24 h after cell transfer with 200 μL containing 10 μg TM4 core (McGuire et al., 2014) or 10mut (Steichen et al., 2016) in PBS with 50% Ribi (Sigma Adjuvant system, Sigma Aldrich) prepared to the manufacturer's instructions. Mice were bled at the indicated time points from the submandibular vein. Blood was allowed to clot and then serum was separated by centrifugation for 10 min at 20817 g. Serum was stored at −20° C.

Anti-Idiotypic Antibody

IgG producing hybridomas were isolated from mice immunized with iGL-VRC01 at the Frederick Hutchinson Cancer Research Center Antibody Technology Resource. Hybridoma supernatants were screened against a matrix of inferred germline (iGL) VRC01 class antibodies as well as irrelevant iGL-antibodies using a high throughput bead-based assay. One anti-idiotypic antibody, clone iv8, bound to additional VRC01 class antibodies, but it also bound to a chimeric antibody with an iGL-VRC01 class light chain paired with the 8ANC131 heavy chain (which is derived from VH1-46), and to 3BNC60^{SI}.

ELISAs

For determination of 3BNC60^{SI} levels, Corning 3690 half-well 96-well plates were coated overnight at 4° C. with 25 μL/well of 2 μg/mL human anti-3BNC60SI (clone iv8) IgG in PBS, then blocked with 150 μL/well PBS 5% skimmed milk for 2 h at room temperature (RT). Sera were diluted to 1:50 with PBS and 7 subsequent 3-fold dilutions. Recombinant 3BNC60SI (produced in house as mouse IgG1,κ) was diluted to 10 μg/mL in PBS followed by six 5-fold dilutions. Blocked plates were washed 4-times with PBS 0.05% Tween 20 and incubated with 25 μL diluted sera or antibody for 2 h at RT. Binding was revealed by either anti-mouse IgG-horseradish peroxidase (HRP) (Jackson ImmunoResearch, Cat. #115-035-071) or anti-mouse IgG1a-biotin (BD Pharmingen Cat. #553500) or anti-mouse IgGlb-biotin (BD Pharmingen Cat. #553533), all diluted 1:5000 in PBS, 25 μL/well and incubation for 1 h at RT. Biotinylated antibodies were subsequently incubated with Streptavidin-HRP (BD Pharmingen Cat. #554066), diluted 1:1000 in PBS, 25 μL/well for 30 min at RT. Plates were washed 4-times with PBS 0.05% Tween 20 in between steps and 6 times before addition of substrate using a Tecan Hydrospeed microplate washer. HRP activity was determined using TMB as substrate (Thermo Scientific Cat. #34021), adding 50 μL/well. Reactions were stopped with 50 μL/well 2 M H2SO4 and read at 450 and 570 nm on a FLUOstar Omega microplate reader (BMG Labtech). Data were analyzed with Microsoft Excel and GraphPad Prism 6.0. Absolute 3BNC60SI titers were interpolated from sigmoidal fits of recombinant 3BNC60^{SI} standard curves.

Determination of human FIX followed the same protocol but wells were coated with 2 ug/mL anti-hF9 (ThermoFisher MA1-43012). Cell culture supernatants were incubated for 2 h and a standard curve was prepared using serial dilutions of recombinant hF9 (R&D 9260-SE-020) in 1% BSA 0.05% Tween 20 starting at 10 ug/mL. Detection was performed using 1 ug/mL HRP-conjugated anti-hF9 polyclonal Ab (Affinity Biologicals, SAFIX APHRP) in 1% BSA 0.05% Tween 20.

For determination of NP-binding antibodies the following modifications applied. Plates were coated with 10 μg/mL NP31-bovine serum albumin (BSA, Biosearch Technologies) and blocked with PBS 3% BSA. Sera, antibodies and secondary reagents were diluted in PBS 1% BSA 0.05% Tween20.

Neutralization Assays

Collected mouse serum was pooled and IgG purified using protein G Ab

SpinTraps (GE Healthcare Cat. #28-4083-47) then concentrated and buffer-exchanged into PBS using Amicon Ultra 30K centrifugal filter units (Merck Millipore Cat. #UFC503024) according to the manufacturers' instructions.

Human FIX Chromogenic Activity Assay

For Human FIX activity in cell culture, cell culture medium was changed 24 h after transfection to medium with 2% FCS and supplemented with 5 ug/mL vitamin K (Sigma-Aldrich Cat #V3501)). Activity in supernatant was determined according to manufacturer's instructions using Rox Factor IX kit (diaPharma Cat #900020) with Technoclone Factor IX Deficient Plasma (diaPharma Cat #5164003) as negative control.

TZM-bl assays were performed as previously described (Montefiori, 2005). Neutralizing activity was calculated as a function of the reduction in Tat-inducible luciferase expression in the TZM-bl reporter cell line in a single round of virus infection.

Additional Information:

FIG. 8 shows that B cells cultured and stimulated as for RNP transfection are able to participate in GCs and produce antibodies. FIG. 9 relates to the choice of murine IgH crRNAs and production of HDRTs. FIG. 10 provides data on murine B cell viability after transfection, Igh allelic exclusion and a promoterless HDRT to improve allelic exclusion. FIG. 11 relates to the choice of human crRNAs and viability of human B ells after transfection. FIG. 12 provides details and additional data of neutralization assays. Table 1 lists crRNA sequences. Table 2 contains annotated HDRT sequences. Table 3 contains primer sequences for TIDE assay and Table 4 details flow cytometric reagents.

### Example 5

As discussed above, the present disclosure provides compositions and methods for passenger protein expression by B cell engineering. The presently provided approaches include multiplexed in vitro targeting of 2-3 loci and introduction of a template for therapeutic protein and novel antibody expression in mature B cells. Following gene-editing, B cells are transferred back in vivo and activated by cognate antigen immunization which leads to expression of the novel antibody and therapeutic protein in the blood. In embodiments, and as discussed above, CRISPR gene editing will target the IgH locus for insertion of the antibody and light chain loci to delete the endogenous κ light chain and λ light chains.

The disclosure includes expression of the antibody to the IgH locus as an artificial polycistronic exon located in between the last J gene and enhancer element.

The therapeutic protein will either be expressed as a cistron from this new exon or it will be inserted into the IgKC locus replacing expression of the kappa light chain (FIG. 5).

B cells are activated in vitro and gene editing of antibody locus performed using CRISPR or any other gene editing method. Modified B cells that express the therapeutic protein and antibody of choice will be transferred into an animal followed by immunization with cognate antigen to ensure activation of edited B cells, their division and maturation into protein secreting plasma cells in vivo.

Antibody locus expression in activated B cells and plasma cells will produce large quantities of the passenger therapeutic protein. Expression will be long lived because plasma cells are long lived. An “on switch” for expression, e.g., expression can be boosted by repeated cognate antigen immunization. An “off switch” for expression, e.g., Anti-idiotype antibodies will delete B cells expressing the antibody that was introduced by gene editing.

The production of therapeutic protein and antibody will be monitored by measuring protein levels or activity in the serum. Experimental protocols presented below are for Factor IX and the B1-8^{hi} antibody as examples of a therapeutic protein antibody pair

The following materials and methods were used to produce results described in this disclosure.

crRNA Design

crRNAs are designed with the Massachusetts Institute of Technology guide design tool (chopchop.cbu.uib.no), and the Integrated DNA Technologies crRNA design tool (www.idtdna.com). Designs are synthesized by Integrated DNA Technologies as Alt-R CRISPR/Cas9 sgRNAs.

Template Production

Templates are synthesized as gBlocks by Integrated DNA Technologies. Packaging vectors for AAV production are cloned using NheI and XhoI restriction sites in pAAV (gift of Paul Bieniasz). Maxi preparations of plasmids are packaged into AAV6 by Vigene. Alternatively, templates are cloned into pLSODN4D using NheI and XhoI restriction sites for ssDNA production as previously described (Hartweger 2019) or created by high-fidelity PCR for dsDNA. Alternative methods such as small plasmids or minicircles might be used instead.

B Cell Purification, Activation and In Vitro Culture

Mature, resting B cells are obtained from mouse spleens in this example but would be obtained from blood in humans. For mouse spleen cells, they will be purified by forcing tissue through a 70-μm mesh into PBS containing 2% heat-inactivated fetal bovine serum (FBS). After ammonium-chloride-potassium buffer lysis for 3 min, untouched B cells are enriched using anti-CD43 magnetic beads according to the manufacturer's protocol (Miltenyi Biotec) obtaining >95% purity. 1-3.2×10⁷ cells/10 cm dish (Gibco) are cultured at 37° C. 5% CO2 in 10 ml mouse B cell medium consisting of RPMI-1640, supplemented with 10% heat-inactivated FBS, 10 mM Hepes, antibiotic-antimycotic (1×), 1 mM sodium pyruvate, 2 mM L-glutamine, and 53 μM 2-mercaptoethanol (all from Gibco) and activated with 2 μg/ml anti-mouse RP105 clone RP/14 (produced in house or 562191; BD Pharmingen). Other stimulants might be used or added to increase efficiency. Human B cells will be obtained from PBMC by magnetic negative depletion and cultured similarly using human reagents (Hartweger 2019).

Gene Editing

This example is for CRISPR based gene editing but any other method for homologous introduction DNA into cells could also be used. Cells are harvest after culture, washed once in PBS and then resuspended at a concentration of 5×10⁵/90 μL in Neon buffer T (Thermo Fisher). Per 100 μl transfection for multiplex targeting of IgH, IgK and IgL, 2.5 μl of each 100 μM sgRNA in duplex buffer (all from Integrated DNA Technologies) are mixed, with 1.33 μl 61 μM Cas9 V3 (1081059; Integrated DNA Technologies) and incubated for 15-30 min., RNPs for different IgH, IgK and IgL loci are mixed at a 1:1:1 ratio. 11.5 μl of RNP mix is mixed with 90 uL of cells in buffer T and transfected in the Neon electroporator at 1650 V 20 ms 1p according to the manufacturer's instructions.

Cells are then transferred into prewarmed culture medium with stimulants as above supplemented with recombinant AAV6 containing the template at an MOI of 0.5-1×10⁶. Alternatively, purified DNA can be transfected along with the RNP as previously described (Hartweger 2019). Reagents and machinery are subject to change to allow better editing efficiencies and GMP manufacturing.

Cell Transfers and Immunizations

Immunization is with the cognate antigen for the antibody that was introduced above. The following is an example for the mouse system but is also applicable to human following appropriate modification for culture of human cells as described in the art. After culture, B cells are harvested at the indicated time points and resuspended in B cell medium without stimulants and rested for 2-3 h at 37° C., 5% CO2. Then cells are washed once in PBS and resuspended in 200 μl PBS/mouse containing the indicated number of initially transfected cells. 200 μl cell suspension/mouse are injected intravenously via the retroorbital sinus. Mice that are deficient for therapeutic protein of choice (for example Factor IX-deficient mice) are used as recipients. The number of transferred, edited B cells is estimated as follows: number of cells transfected×20% survival×10% transfection efficiency ×50% handling/proliferation ×5% transfer efficiency (Dosenovic et al., 2018). Mice are immunized intraperitoneally within a week after cell transfer with cognate antigen such as NP-OVA in PBS (Biosearch Technologies) for antibody B1-8^{hi}. Immunization can be adjuvanted by 50% Ribi (Sigma Adjuvant system; Sigma-Aldrich) prepared according to the manufacturer's instructions or other appropriate adjuvants. Mice are bled at the indicated time points from the submandibular vein. Blood is allowed to clot, and then serum is separated by centrifugation for 10 min at 20,817 g. Serum is stored at −20° C.

ELISAs

For determination of the B1-8 antibody levels, Corning 3690 half-well 96-well plates are coated overnight at 4° C. with 25 μl/well of 10 μg/ml NP₃₁-BSA (Biosearch Technologies) then blocked with 150 μl/well PBS 3% skimmed milk for 2 h at room temperature (RT). Sera is diluted to 1:50 with PBS and seven subsequent threefold dilutions. Blocked plates are washed four times with PBS 0.05% Tween 20 and incubated with 25 μl diluted sera or antibody for 2 h at RT. Binding is revealed by anti-mouse IgG-HRP (115-035-071; Jackson ImmunoResearch), diluted 1:5,000 in PBS, 25 μl/well, and incubated for 1 h at RT. Biotinylated antibodies are subsequently incubated with streptavidin-HRP (554066; BD Pharmingen) 25 μl/well, for 30 min at RT. Plates are washed four times with PBS 0.05% Tween 20 in between steps and six times before addition of substrate using a Tecan Hydrospeed microplate washer. HRP activity is determined using 3,3′,5,5′ tetramethylbenzidine as substrate (34021; Thermo Fisher Scientific), adding 50 μl/well. Reactions are stopped with 50 μl/well 2 M H2504 and read at 450 and 570 nm on a FLUOstar Omega microplate reader (BMG Labtech). Data are analyzed with Microsoft Excel and GraphPad Prism 6.0.

The level of factor IX in the plasma is determined using Mouse Factor IX ELISA kit from LSBIO Cat. #LS-F4177 as per manufacturer's instructions.

### REFERENCES

This reference listing is not an indication that any particular reference is material to patentability:
Hartweger et al. 2019. J Exp Med. 216(6):1301-1310. HIV-specific humoral immune responses by CRISPR/Cas9-edited B cells. doi: 10.1084/jem.20190287.
Dosenovic et al. 2018 PNAS 115(18):4743-4748. Anti-HIV-1 B cell responses are dependent on B cell precursor frequency and antigen-binding affinity. doi: 10.1073/pnas.1803457115
Bar, K. J., M. C. Sneller, L. J. Harrison, J. S. Justement, E. T. Overton, M. E. Petrone, D. B. Salantes, C. A. Seamon, B. Scheinfeld, R. W. Kwan, G. H. Learn, M. A. Proschan, E. F. Kreider, J. Blazkova, M. Bardsley, E. W. Refsland, M. Messer, K. E. Clarridge, N. B. Tustin, P. J. Madden, K. Oden, S. J. O'Dell, B. Jarocki, A. R. Shiakolas, R. L. Tressler, N. A. Doria-Rose, R. T. Bailer, J. E. Ledgerwood, E. V. Capparelli, R. M. Lynch, B. S. Graham, S. Moir, R. A. Koup, J. R. Mascola, J. A. Hoxie, A. S. Fauci, P. Tebas, and T. W. Chun. 2016. Effect of HIV Antibody VRC01 on Viral Rebound after Treatment Interruption. N Engl J Med 375:2037-2050.
Briney, B., D. Sok, J. G. Jardine, D. W. Kulp, P. Skog, S. Menis, R. Jacak, O. Kalyuzhniy, N. de Val, F. Sesterhenn, K. M. Le, A. Ramos, M. Jones, K. L. Saye-Francisco, T. R. Blane, S. Spencer, E. Georgeson, X. Hu, G. Ozorowski, Y. Adachi, M. Kubitz, A. Sarkar, I. A. Wilson, A. B. Ward, D. Nemazee, D. R. Burton, and W. R. Schief. 2016. Tailored Immunogens Direct Affinity Maturation toward HIV Neutralizing Antibodies. Cell 166:1459-1470.e1411.
Brinkman, E. K., T. Chen, M. Amendola, and B. van Steensel. 2014. Easy quantitative assessment of genome editing by sequence trace decomposition. Nucleic Acids Res 42:e168. Caskey, M., F. Klein, J. C. Lorenzi, M. S. Seaman, A. P. West, N. Buckley, G. Kremer, L. Nogueira, M. Braunschweig, J. F. Scheid, J. A. Horwitz, I. Shimeliovich, S. Ben-Avraham, M. Witmer-Pack, M. Platten, C. Lehmann, L. A. Burke, T. Hawthorne, R. J. Gorelick, B. D. Walker, T. Keler, R. M. Gulick, G. Fatkenheuer, S. J. Schlesinger, and M. C. Nussenzweig. 2015. Viraemia suppressed in HIV-1-infected humans by broadly neutralizing antibody 3BNC117. Nature 522:487-491.
Caskey, M., T. Schoofs, H. Gruell, A. Settler, T. Karagounis, E. F. Kreider, B. Murrell, N. Pfeifer, L. Nogueira, T. Y. Oliveira, G. H. Learn, Y. Z. Cohen, C. Lehmann, D. Gillor, I. Shimeliovich, C. Unson-O'Brien, D. Weiland, A. Robles, T. Kummerle, C. Wyen, R. Levin, M. Witmer-Pack, K. Eren, C. Ignacio, S. Kiss, A. P. West, H. Mouquet, B. S. Zingman, R. M. Gulick, T. Keler, P. J. Bjorkman, M. S. Seaman, B. H. Hahn, G. Fatkenheuer, S. J. Schlesinger, M. C. Nussenzweig, and F. Klein. 2017. Antibody 10-1074 suppresses viremia in HIV-1-infected individuals. Nat Med 23:185-191.
Cebra, J. J., J. E. Colberg, and S. Dray. 1966. Rabbit lymphoid cells differentiated with respect to alpha-, gamma-, and mu- heavy polypeptide chains and to allotypic markers Aa1 and Aa2. J Exp Med 123:547-558.
Dosenovic, P., E. E. Kara, A. K. Pettersson, A. T. McGuire, M. Gray, H. Hartweger, E. S. Thientosapol, L. Stamatatos, and M. C. Nussenzweig. 2018. Anti-HIV-1 B cell responses are dependent on B cell precursor frequency and antigen-binding affinity. Proc Natl Acad Sci USA 115:4743-4748.
Dosenovic, P., L. von Boehmer, A. Escolano, J. Jardine, N. T. Freund, A. D. Gitlin, A. T. McGuire, D. W. Kulp, T. Oliveira, L. Scharf, J. Pietzsch, M. D. Gray, A. Cupo, M. J. van Gils, K. H. Yao, C. Liu, A. Gazumyan, M. S. Seaman, P. J. Bjorkman, R. W. Sanders, J. P. Moore, L. Stamatatos, W. R. Schief, and M. C. Nussenzweig. 2015. Immunization for HIV-1 Broadly Neutralizing Antibodies in Human Ig Knockin Mice. Cell 161:1505-1515.
Escolano, A., P. Dosenovic, and M. C. Nussenzweig. 2017. Progress toward active or passive HIV-1 vaccination. J Exp Med 214:3-16.
Escolano, A., J. M. Steichen, P. Dosenovic, D. W. Kulp, J. Golijanin, D. Sok, N. T. Freund, A. D. Gitlin, T. Oliveira, T. Araki, S. Lowe, S. T. Chen, J. Heinemann, K. H. Yao, E. Georgeson, K. L. Saye-Francisco, A. Gazumyan, Y. Adachi, M. Kubitz, D. R. Burton, W. R. Schief, and M. C. Nussenzweig. 2016. Sequential Immunization Elicits Broadly Neutralizing Anti-HIV-1 Antibodies in Ig Knockin Mice. Cell 166:1445-1458.e1412.
Eyquem, J., J. Mansilla-Soto, T. Giavridis, S. J. van der Stegen, M. Hamieh, K. M. Cunanan, A. Odak, M. Gönen, and M. Sadelain. 2017. Targeting a CAR to the TRAC locus with CRISPR/Cas9 enhances tumour rejection. Nature 543:113-117.
Freitag, J., S. Heink, E. Roth, J. Wittmann, H. M. Jack, and T. Kamradt. 2014. Towards the generation of B-cell receptor retrogenic mice. PLoS One 9:e109199.
Jacobsen, J. T., L. Mesin, S. Markoulaki, A. Schiepers, C. B. Cavazzoni, D. Bousbaine, R. Jaenisch, and G. D. Victora. 2018. One-step generation of monoclonal B cell receptor mice capable of isotype switching and somatic hypermutation. J Exp Med 215:2686-2695.
Kraus, M., M. B. Alimzhanov, N. Raj ewsky, and K. Raj ewsky. 2004. Survival of resting mature B lymphocytes depends on BCR signaling via the Igalpha/beta heterodimer. Cell 117:787-800.
Kuraoka, M., A. G. Schmidt, T. Nojima, F. Feng, A. Watanabe, D. Kitamura, S. C. Harrison, T. B. Kepler, and G. Kelsoe. 2016. Complex Antigens Drive Permissive Clonal Selection in Germinal Centers. Immunity 44:542-552.
Kwong, P. D., and J. R. Mascola. 2018. HIV-1 Vaccines Based on Antibody Identification, B Cell Ontogeny, and Epitope Structure. Immunity 48:855-871. Lam, K. P., R. Kühn, and K. Raj ewsky. 1997. In vivo ablation of surface immunoglobulin on mature B cells by inducible gene targeting results in rapid cell death. Cell 90:1073-1083.
Ledgerwood, J. E., E. E. Coates, G. Yamshchikov, J. G. Saunders, L. Holman, M. E. Enama, A. DeZure, R. M. Lynch, I. Gordon, S. Plummer, C. S. Hendel, A. Pegu, M. Conan-Cibotti, S. Sitar, R. T. Bailer, S. Narpala, A. McDermott, M. Louder, S. O'Dell, S. Mohan, J. P. Pandey, R. M. Schwartz, Z. Hu, R. A. Koup, E. Capparelli, J. R. Mascola, B. S. Graham, and V. S. Team. 2015. Safety, pharmacokinetics and neutralization of the broadly neutralizing HIV-1 human monoclonal antibody VRC01 in healthy adults. Clin Exp Immunol 182:289-301.
Lim, W. A., and C. H. June. 2017. The Principles of Engineering Immune Cells to Treat Cancer. Cell 168:724-740.
Lynch, R. M., E. Boritz, E. E. Coates, A. DeZure, P. Madden, P. Costner, M. E. Enama, S. Plummer, L. Holman, C. S. Hendel, I. Gordon, J. Casazza, M. Conan-Cibotti, S. A. Migueles, R. Tressler, R. T. Bailer, A. McDermott, S. Narpala, S. O'Dell, G. Wolf, J. D. Lifson, B. A. Freemire, R. J. Gorelick, J. P. Pandey, S. Mohan, N. Chomont, R. Fromentin, T. W. Chun, A. S. Fauci, R. M. Schwartz, R. A. Koup, D. C. Douek, Z. Hu, E. Capparelli, B. S. Graham, J. R. Mascola, J. E. Ledgerwood, and V. S. Team. 2015. Virologic effects of broadly neutralizing antibody VRC01 administration during chronic HIV-1 infection. Sci Transl Med 7:319ra206.
Mascola, J. R., M. G. Lewis, G. Stiegler, D. Harris, T. C. VanCott, D. Hayes, M. K. Louder, C. R. Brown, C. V. Sapan, S. S. Frankel, Y. Lu, M. L. Robb, H. Katinger, and D. L. Birx. 1999. Protection of Macaques against pathogenic simian/human immunodeficiency virus 89.6PD by passive transfer of neutralizing antibodies. J Virol 73:4009-4018.
McCoy, L. E., A. F. Quigley, N. M. Strokappe, B. Bulmer-Thomas, M. S. Seaman, D. Mortier, L. Rutten, N. Chander, C. J. Edwards, R. Ketteler, D. Davis, T. Verrips, and R. A. Weiss. 2012. Potent and broad neutralization of HIV-1 by a llama antibody elicited by immunization. J Exp Med 209:1091-1103.
McGuire, A. T., A. M. Dreyer, S. Carbonetti, A. Lippy, J. Glenn, J. F. Scheid, H. Mouquet, and L. Stamatatos. 2014. HIV antibodies. Antigen modification regulates competition of broad and narrow neutralizing HIV antibodies. Science 346:1380-1383.
McGuire, A. T., M. D. Gray, P. Dosenovic, A. D. Gitlin, N. T. Freund, J. Petersen, C. Correnti, W. Johnsen, R. Kegel, A. B. Stuart, J. Glenn, M. S. Seaman, W. R. Schief, R. K. Strong, M. C. Nussenzweig, and L. Stamatatos. 2016. Specifically modified Env immunogens activate B-cell precursors of broadly neutralizing HIV-1 antibodies in transgenic mice. Nat Commun 7:10618.
Mendoza, P., H. Gruell, L. Nogueira, J. A. Pai, A. L. Butler, K. Millard, C. Lehmann, I. Suárez, T. Y. Oliveira, J. C. C. Lorenzi, Y. Z. Cohen, C. Wyen, T. Kümmerle, T. Karagounis, C. L. Lu, L. Handl, C. Unson-O'Brien, R. Patel, C. Ruping, M. Schlotz, M. Witmer-Pack, I. Shimeliovich, G. Kremer, E. Thomas, K. E. Seaton, J. Horowitz, A. P. West, P. J. Bjorkman, G. D. Tomaras, R. M. Gulick, N. Pfeifer, G. Fatkenheuer, M. S. Seaman, F. Klein, M. Caskey, and M. C. Nussenzweig. 2018. Combination therapy with anti-HIV-1 antibodies maintains viral suppression. Nature 561:479-484.
Miura, Y., R. Shimazu, K. Miyake, S. Akashi, H. Ogata, Y. Yamashita, Y. Narisawa, and M. Kimoto. 1998. RP105 is associated with MD-1 and transmits an activation signal in human B cells. Blood 92:2815-2822.
Montefiori, D. C. 2005. Evaluating neutralizing antibodies against HIV, SIV, and SHIV in luciferase reporter gene assays. Curr Protoc Immunol Chapter 12:Unit 12.11.
Mouquet, H., L. Scharf, Z. Euler, Y. Liu, C. Eden, J. F. Scheid, A. Halper-Stromberg, P. N. Gnanapragasam, D. I. Spencer, M. S. Seaman, H. Schuitemaker, T. Feizi, M. C. Nussenzweig, and P. J. Bjorkman. 2012. Complex-type N-glycan recognition by potent broadly neutralizing HIV antibodies. Proc Natl Acad Sci USA 109:E3268-3277.
Nishimura, Y., and M. A. Martin. 2017. Of Mice, Macaques, and Men: Broadly Neutralizing Antibody Immunotherapy for HIV-1. Cell Host Microbe 22:207-216. Nussenzweig, M. C., A. C. Shaw, E. Sinn, D. B. Danner, K. L. Holmes, H. C. Morse, and P. Leder. 1987. Allelic exclusion in transgenic mice that express the membrane form of immunoglobulin mu. Science 236:816-819.
Parren, P. W., P. A. Marx, A. J. Hessell, A. Luckay, J. Harouse, C. Cheng-Mayer, J. P. Moore, and D. R. Burton. 2001. Antibody protects macaques against vaginal challenge with a pathogenic R5 simian/human immunodeficiency virus at serum levels giving complete neutralization in vitro. J Virol 75:8340-8347.
Pernis, B., G. Chiappino, A. S. Kelus, and P. G. Gell. 1965. Cellular localization of immunoglobulins with different allotypic specificities in rabbit lymphoid tissues. J Exp Med 122:853-876.
Roth, T. L., C. Puig-Saus, R. Yu, E. Shifrut, J. Carnevale, P. J. Li, J. Hiatt, J. Saco, P. Krystofinski, H. Li, V. Tobin, D. N. Nguyen, M. R. Lee, A. L. Putnam, A. L. Ferris, J. W. Chen, J. N. Schickel, L. Pellerin, D. Carmody, G. Alkorta-Aranburu, D. Del Gaudio, H. Matsumoto, M. Morell, Y. Mao, M. Cho, R. M. Quadros, C. B. Gurumurthy, B. Smith, M. Haugwitz, S. H. Hughes, J. S. Weissman, K. Schumann, J. H. Esensten, A. P. May, A. Ashworth, G. M. Kupfer, S. A. W. Greeley, R. Bacchetta, E. Meffre, M. G. Roncarolo, N. Romberg, K. C. Herold, A. Ribas, M. D. Leonetti, and A. Marson. 2018. Reprogramming human T cell function and specificity with non-viral genome targeting. Nature 559:405-409.
Sadelain, M., I. Rivière, and S. Riddell. 2017. Therapeutic T cell engineering. Nature 545:423-431.
Scheid, J. F., J. A. Horwitz, Y. Bar-On, E. F. Kreider, C. L. Lu, J. C. Lorenzi, A. Feldmann, M. Braunschweig, L. Nogueira, T. Oliveira, I. Shimeliovich, R. Patel, L. Burke, Y. Z. Cohen, S. Hadrigan, A. Settler, M. Witmer-Pack, A. P. West, B. Juelg, T. Keler, T. Hawthorne, B. Zingman, R. M. Gulick, N. Pfeifer, G. H. Learn, M. S. Seaman, P. J. Bjorkman, F. Klein, S. J. Schlesinger, B. D. Walker, B. H. Hahn, M. C. Nussenzweig, and M. Caskey. 2016. HIV-1 antibody 3BNC117 suppresses viral rebound in humans during treatment interruption. Nature 535:556-560.
Schoofs, T., F. Klein, M. Braunschweig, E. F. Kreider, A. Feldmann, L. Nogueira, T. Oliveira, J. C. Lorenzi, E. H. Parrish, G. H. Learn, A. P. West, P. J. Bjorkman, S. J. Schlesinger, M. S. Seaman, J. Czartoski, M. J. McElrath, N. Pfeifer, B. H. Hahn, M. Caskey, and M. C. Nussenzweig. 2016. HIV-1 therapy with monoclonal antibody 3BNC117 elicits host immune responses against HIV-1. Science 352:997-1001.
Shibata, R., T. Igarashi, N. Haigwood, A. Buckler-White, R. Ogert, W. Ross, R. Willey, M. W. Cho, and M. A. Martin. 1999. Neutralizing antibody directed against the HIV-1 envelope glycoprotein can completely block HIV-1/SIV chimeric virus infections of macaque monkeys. Nat Med 5:204-210.
Shih, T. A., M. Roederer, and M. C. Nussenzweig. 2002. Role of antigen receptor affinity in T cell-independent antibody responses in vivo. Nat Immunol 3:399-406. Shingai, M., O. K. Donau, R. J. Plishka, A. Buckler-White, J. R. Mascola, G. J. Nabel, M. C. Nason, D. Montefiori, B. Moldt, P. Poignard, R. Diskin, P. J. Bjorkman, M. A. Eckhaus, F. Klein, H. Mouquet, J. C. Cetrulo Lorenzi, A. Gazumyan, D. R. Burton, M. C. Nussenzweig, M. A. Martin, and Y. Nishimura. 2014. Passive transfer of modest titers of potent and broadly neutralizing anti-HIV monoclonal antibodies block SHIV infection in macaques. J Exp Med 211:2061-2074.
Sok, D., and D. R. Burton. 2018. Recent progress in broadly neutralizing antibodies to HIV. Nat Immunol 19:1179-1188.
Sok, D., K. M. Le, M. Vadnais, K. L. Saye-Francisco, J. G. Jardine, J. L. Torres, Z. T. Berndsen, L. Kong, R. Stanfield, J. Ruiz, A. Ramos, C. H. Liang, P. L. Chen, M. F. Criscitiello, W. Mwangi, I. A. Wilson, A. B. Ward, V. V. Smider, and D. R. Burton. 2017. Rapid elicitation of broadly neutralizing antibodies to HIV by immunization in cows. Nature 548:108-111. Steichen, J. M., D. W. Kulp, T. Tokatlian, A. Escolano, P. Dosenovic, R. L. Stanfield, L. E. McCoy, G. Ozorowski, X. Hu, O. Kalyuzhniy, B. Briney, T. Schiffner, F. Garces, N. T. Freund, A. D. Gitlin, S. Menis, E. Georgeson, M. Kubitz, Y. Adachi, M. Jones, A. A. Mutafyan, D. S. Yun, C. T. Mayer, A. B. Ward, D. R. Burton, I. A. Wilson, D. J. Irvine, M. C. Nussenzweig, and W. R. Schief. 2016. HIV Vaccine Design to Target Germline Precursors of Glycan-Dependent Broadly Neutralizing Antibodies. Immunity 45:483-496.
Tian, M., C. Cheng, X. Chen, H. Duan, H. L. Cheng, M. Dao, Z. Sheng, M. Kimble, L. Wang, S. Lin, S. D. Schmidt, Z. Du, M. G. Joyce, Y. Chen, B. J. DeKosky, E. Normandin, E. Cantor, R. E. Chen, N. A. Doria-Rose, Y. Zhang, W. Shi, W. P. Kong, M. Choe, A. R. Henry, F. Laboune, I. S. Georgiev, P. Y. Huang, S. Jain, A. T. McGuire, E. Georgeson, S. Menis, D. C. Douek, W. R. Schief, L. Stamatatos, P. D. Kwong, L. Shapiro, B. F. Haynes, J. R. Mascola, and F. W. Alt. 2016. Induction of HIV Neutralizing Antibody Lineages in Mice with Diverse Precursor Repertoires. Cell 166:1471-1484.e1418.
Voss, J. E., A. Gonzalez-Martin, R. Andrabi, R. P. Fuller, B. Murrell, L. E. McCoy, K. Porter, D. Huang, W. Li, D. Sok, K. Le, B. Briney, M. Chateau, G. Rogers, L. Hangartner, A. J. Feeney, D. Nemazee, P. Cannon, and D. Burton. 2019. Reprogramming the antigen specificity of B cells using genome-editing technologies. Elife 8:
Yoshimi, K., Y. Kunihiro, T. Kaneko, H. Nagahora, B. Voigt, and T. Mashimo. 2016. ssODN-mediated knock-in with CRISPR-Cas for large genomic regions in zygotes. Nat Commun 7:10431.

Although the subject matter of this disclosure has been described above in terms of certain embodiments/examples, other embodiments/examples, including embodiments/examples that do not provide all of the benefits and features set forth herein, are also within the scope of this disclosure. Various structural, logical, and process step changes may be made without departing from the scope of the disclosure.

1. A modified B cell comprising:
i) a single contiguous DNA sequence encoding a heterologous antibody, the heterologous antibody comprising a variable light chain region, an antibody light chain constant region, and an antibody variable heavy chain region, said sequence being introduced into an IgH locus in the B cell, and wherein said sequence comprises a sequence encoding a cargo protein that is co-expressed with the heterologous antibody from a polycistronic element that also encodes the cargo protein, a variable light chain region, a light chain constant region and a variable heavy chain region, and wherein a λ light chain locus in the modified B cells is deleted or disrupted, and wherein optionally the IgK locus in the B cells is separately disrupted or deleted; or
ii) a first contiguous DNA sequence encoding a heterologous antibody, the heterologous antibody comprising a variable light chain region, a light chain constant region and a variable heavy chain region, said first contiguous DNA sequence having been introduced into the IgH locus, and a second contiguous DNA sequence encoding a cargo protein, wherein said second sequence encoding the cargo protein is introduced into the IgK locus, and wherein the λ light chain locus is also disrupted or deleted.

2. The modified B cell of claim 1, wherein the modified B cell comprises i) of claim 1.

3. The modified B cell of claim 2, wherein the single contiguous DNA sequence is homologously recombined into the IgH locus, and optionally in an IgH locus position that is between a J_{H} and an E_{μ} enhancer segment, and comprises in a 5′ to 3′ orientation:
a) a first homology arm used for homologous recombination into the IgH locus;
b) a splice acceptor;
c) a first ribosome skipping sequence;
d) a sequence encoding the cargo protein;
e) a second ribosome skipping sequence;
f) the variable light chain region;
g) a variable light chain constant region;
h) a third ribosome skipping sequence;
i) the heavy chain variable sequence;
j) a splice donor; and
k) a second homology arm used for recombination into the IgH locus.

4. The modified B cell of claim 1, wherein the modified B cell comprises ii) of claim 1.

5. The modified B cell of claim 3, wherein the first contiguous sequence encoding the heterologous antibody is homologously recombined into the IgH locus, and comprises in a 5′ to 3′ orientation:
a) a first homology arm used for homologous recombination into the IgH locus;
b) a splice acceptor;
c) a first ribosome skipping sequence;
d) the variable light chain region;
e) a variable light chain constant region;
f) a second ribosome skipping sequence;
g) the heavy chain variable sequence;
h) a splice donor; and
i) a second homology arm used for recombination in to the IgH locus;
and wherein the second contiguous sequence encoding the cargo protein is homologously recombined into the IgK locus and comprises in a 5′ to 3′ orientation:
j) a third homology arm used for recombination into the IgK locus;
k) a first Cκ segment, which may be all or a part of the homology arm which places a 3′ sequence with a Cκ exon;
l) a ribosome skipping sequence;
m) a sequence encoding the cargo protein;
n) a stop codon;
o) a second Cκ segment; and
p) a fourth homology arm used for recombination into the IgK locus.

6. The modified B cell of claim 1, wherein the heterologous antibody is displayed on the surface of the modified B cell.

7. The modified B cell of claim 6, wherein the heterologous antibody is bound to an antigen to which that heterologous antibody binds with specificity and thereby activates the B cell.

8. The modified B cell of claim 6, wherein the heterologous antibody is bound to an anti-idiotypic antibody via a paratope of the heterologous antibody.

9. A population of modified B cells comprising modified B cells of claim 1.

10. Plasma cells differentiated from a modified B cell of claim 1.

11. A single stranded DNA molecule comprising in a 5′ to 3′ orientation:
a) a first homology arm used for homologous recombination into the IgH locus;
b) a splice acceptor;
c) a first ribosome skipping sequence;
d) a sequence encoding the cargo protein;
e) a second ribosome skipping sequence;
f) the variable light chain region;
g) a variable light chain constant region;
h) a third ribosome skipping sequence;
i) the heavy chain variable sequence;
j) a splice donor; and
k) a second homology arm used for recombination into the IgH locus;
or
a.1) a first homology arm used for homologous recombination into the IgH locus;
b.1) a splice acceptor;
c.1) a first ribosome skipping sequence;
d.1) the variable light chain region;
e.1) a variable light chain constant region;
f.1) a second ribosome skipping sequence;
g.1) the heavy chain variable sequence;
h.1) a splice donor; and
i.1) a second homology arm used for recombination in to the IgH locus;
or
j) a third homology arm used for recombination into the IgK locus;
k) a first Cκ segment, which may be all or a part of the homology arm which places a 3′ sequence with a Cκ exon;
l) a ribosome skipping sequence;
m) a sequence encoding the cargo protein;
n) a stop codon;
o) a second Cκ segment and
p) a fourth homology arm used for recombination into the IgK locus.

12. The single stranded DNA molecule of claim 11, comprising
a) a first homology arm used for homologous recombination into the IgH locus;
b) a splice acceptor;
c) a first ribosome skipping sequence;
d) a sequence encoding the cargo protein;
e) a second ribosome skipping sequence;
f) the variable light chain region;
g) a variable light chain constant region;
h) a third ribosome skipping sequence;
i) the heavy chain variable sequence;
j) a splice donor; and
k) a second homology arm used for recombination into the IgH locus.

13. The single stranded DNA molecule of claim 11, comprising
j) a third homology arm used for recombination into the IgK locus;
k) a first Cκ segment, which may be all or a part of the homology arm which places a 3′ sequence with a Cκ exon;
l) a ribosome skipping sequence;
m) a sequence encoding the cargo protein;
n) a stop codon;
o) a second Cκ segment and
p) a fourth homology arm used for recombination into the IgK locus.

14. A combination of single stranded DNA molecules of claim 12.

15. A method of making a modified B cell of claim 1, the method comprising:
introducing into the B cell at least one Cas protein, guide RNAs that facilitate, in conjunction with the Cas protein, homologous recombination of the single contiguous DNA segment of i) of claim 1, such that a sequence encoding the heterologous antibody comprising the variable light chain region, the constant region, and the variable heavy chain region, is introduced into the IgH locus, and wherein the sequence encoding the protein that is co-expressed with the antibody is also introduced into the IgH locus; and optionally disrupting or deleting the IgK locus, or;
introducing into the B cell least one Cas protein, guide RNAs that facilitate, in conjunction with the Cas protein, homologous recombination of the first contiguous DNA sequence of ii) of claim 1, such that a sequence encoding the heterologous antibody light chain variable, constant, and heavy chain variable region is introduced into the IgH locus, and wherein the second contiguous sequence encoding the cargo protein is introduced into the IgK locus, and wherein the λ light chain locus optionally is also disrupted or deleted.

16. The method of claim 15, wherein the single contiguous DNA segment is recombined into the IgH locus and comprises in a 5′ to 3′ orientation:
a) a first homology arm used for the homologous recombination into the IgH locus;
b) a splice acceptor;
c) a first ribosome skipping sequence;
d) a sequence encoding the cargo protein;
e) a second ribosome skipping sequence;
f) a variable light chain region of the heterologous antibody;
g) a variable light chain constant region of the heterologous antibody;
h) a third ribosome skipping sequence;
i) the heavy chain variable sequence of the heterologous antibody;
j) a splice donor; and
k) a second homology arm used for the homologous recombination into the IgH locus.

17. The method of claim 15, wherein a first contiguous sequence encoding the heterologous antibody is homologously recombined into the IgH locus, and comprises in a 5′ to 3′ orientation:
a) a first homology arm used for the homologous recombination into the IgH locus;
b) a splice acceptor;
c) a first ribosome skipping sequence;
d) a variable light chain region;
e) a variable light chain constant region;
f) a second ribosome skipping sequence;
g) a heavy chain variable sequence;
h) a splice donor; and
i) and a second homology arm that used for the homologous recombination into the IgH locus;
and wherein a second contiguous sequence encoding a cargo protein is homologously recombined into the IgK locus and comprises in a 5′ to 3′ orientation:
j) a third homology arm used for the homologous recombination into the IgK locus;
k) a first Cκ segment, which may be all or a part of the homology arm which places a 3′ sequence with a Cκ exon;
l) a ribosome skipping sequence;
m) a sequence encoding the cargo protein;
n) a stop codon;
o) a second Cκ segment; and
p) a fourth homology arm used for the homologous recombination into the IgK locus.

18. A method comprising introducing a population of modified B cells of claim 9 into an individual.

19. The method of claim 18, further comprising introducing into the individual an antigen comprising an epitope that is specifically recognized by the heterologous antibody expressed by the modified B cells and/or plasma cells differentiated from the modified B cells.

20. The method of claim 19, wherein binding of the antigen to the heterologous antibody promotes expression of the cargo protein.

21. The method of claim 19, wherein the cargo protein provides a therapeutic benefit to the individual.

22. The method of claim 19, further comprising administering to the individual an anti-idiotypic antibody to the individual, the anti-idiotypic antibody having specificity for an idiotype comprised by the heterologous antibody, and wherein said administering the anti-idiotypic antibody reduces or eliminates the modified B cells and/or plasma cells derived from the B cells in the individual.

23. A combination of single stranded DNA molecules of claim 13.
